(19) United States
(12) Patent Application Publication
(10) Pub. No.: US 2023/0154567 A1
(43) Pub. Date: May 18, 2023

(54) A UNIFIED PORTAL FOR REGULATORY AND SPLICING ELEMENTS FOR GENOME ANALYSIS

(71) Applicant: Genome International Corporation, (US)

(72) Inventors: Sudar Senapathy, Madison, WI (US); Periannan Senapathy, Madison, WI (US)

(21) Appl. No.: 17/770,000

(22) PCT Filed: Aug. 20, 2021
(86) PCT No.: PCT/US2021/047025

§ 371 Date: Apr. 18, 2022

(51) Int. Cl. G16B 30/10; G16B 45/00; G16B 40/00

(52) U.S. Cl. CPC G16B 30/10; G16B 45/00; G16B 40/00

(57) ABSTRACT

A method, including identifying, in a nucleotide string, at least two exons, at least one acceptor, at least one donor, and at least one intron between the at least two exons, is provided. The method includes identifying, in the nucleotide string, a cryptic splice site comprising a sequence of nucleotides based on a similarity score with at least one of the acceptor or the donor, and graphically marking, in a display for a user, the nucleotide string at a location indicative of an exon, an intron, a true splice site, and optionally a cryptic splice site when the similarity score is higher than a pre-selected threshold. A system and a non-transitory, computer-readable medium including instructions to cause the system to perform the method are also provided.

### CROSS-REFERENCE TO RELATED APPLICATIONS

The present disclosure claims priority under Article 8 of the PCT to U.S. Provisional Application No. 63/166,803, entitled “A UNIFIED PORTAL FOR REGULATORY AND SPLICING ELEMENTS FOR GENOME ANALYSIS,” to Sudar Senapathy, filed on Mar. 26, 2021, and to U.S. Provisional Application No. 63/166,829, entitled “A PRECISION MEDICINE PORTAL FOR HUMAN DISEASES,” to Periannan Senapathy, filed on Mar. 26, 2021, the contents of both applications incorporated herein by reference in their entirety, for all purposes.

### BACKGROUND

### Field

The present disclosure relates generally to a platform of networked computing devices for performing a comprehensive analysis of gene regulation within the human genome. More specifically, the present disclosure provides a map of genes and mutations thereof for an individual or a cohort of individuals, and their functionality and phenotypic manifestations for use in disease diagnostics and therapeutics and in the analysis of other inherited traits in the human genome.

### Related Art

In the field of genomic analysis, much relevance is given to protein encoding portions of the genome. However, little is known as to other portions of the genome that may not encode proteins, but may be linked to disease and other phenotypic traits yet to be discovered. However, there is a lack of a systematic approach to search, classify, identify, and illustrate coding and non-coding portions of the genome and associated mutations.

### SUMMARY

In a first embodiment, a computer-implemented method includes identifying, in a nucleotide string, at least two exons, at least one acceptor, at least one donor, and at least one intron between the at least two exons, identifying, in the nucleotide string, a cryptic splice site including a sequence of nucleotides based on a similarity score with at least one of the acceptor or the donor, and graphically marking, in a display for a user, the nucleotide string at a location indicative of an exon, an intron, a true splice site, and optionally a cryptic splice site when the similarity score is higher than a pre-selected threshold.

In a second embodiment, a computer-implemented method includes identifying a first amino acid string corresponding to a functional protein or protein domain, aligning said first amino acid string with at least one additional amino acid string that encodes a functional variant of said functional protein, identifying, at each amino acid position within said additional amino acid string, multiple variable amino acids that appear in the at least one additional amino acid string for each aligned location in the first amino acid string, and graphically marking, in a display for a user, a variable amino acid as an allowable amino acid at an aligned location in said first amino acid string.

In a third embodiment, a computer-implemented method includes identifying, in a nucleotide string, at least two exons, and at least one intron between the at least two exons, and a promoter sequence, selecting, within the nucleotide string, a cryptic promoter site including a sequence of nucleotides resembling the promoter sequence, associating a score to the cryptic promoter site based on a similarity score between the cryptic promoter site and the promoter sequence, and graphically marking, in a display for a user, the nucleotide string at a location indicative of the cryptic promoter site when the score is higher than a pre-selected threshold.

In a fourth embodiment, a computer-implemented method includes identifying, in a nucleotide string, a poly-A addition site, wherein the poly-A addition site includes a poly-A site and a signal, selecting, within the nucleotide string, a cryptic poly-A site, the cryptic poly-A site including a sequence of nucleotides resembling at least one of the poly-A sites, associating a similarity score to the cryptic poly-A site based on a similarity between the cryptic poly-A site and a real poly-A site, and graphically marking, in a display for a user, the nucleotide string at a location indicative of the cryptic poly-A site when the similarity score is higher than a pre-selected threshold.

In yet another embodiment, a computer-implemented method includes identifying a first nucleotide string corresponding to a non-coding RNA gene. The computer-implemented method also includes aligning said first nucleotide string with at least one additional nucleotide string that specifies a functional variant of said non-coding RNA gene, and identifying, at each nucleotide position within said additional nucleotide string, multiple variable nucleotides that appear in the at least one additional nucleotide string for each aligned location in the first nucleotide string. The computer-implemented method includes graphically marking, in a display for a user, a variable nucleotide as an allowable nucleotide at an aligned location in said first nucleotide string.

### BRIEF DESCRIPTION OF THE DRAWINGS

FIG. 1 illustrates an architecture of devices and systems for providing a personalized product service, according to some embodiments.

FIG. 2 illustrates the details for devices and systems in the architecture of FIG. 1, according to some embodiments.

FIGS. 3A-3F illustrate details of exon splices, according to embodiments disclosed herein.

FIGS. 4A-4C illustrate details of cryptic splices, according to embodiments disclosed herein.

FIGS. 5A-5C illustrate an exon chart, according to embodiments disclosed herein.

FIGS. 6A-6D illustrate exemplary embodiments of alternative splices, as disclosed herein.

FIGS. 7A-7E illustrate exemplary embodiments of an exon frame, as disclosed herein.

FIGS. 8A-8D illustrate exemplary embodiments of a protein signature, according to embodiments disclosed herein.

FIGS. 9A-9F illustrate exemplary embodiments of an un-translated portion of a genome, according to embodiments disclosed herein.

FIGS. 10A-10B illustrate exemplary embodiments of a branch point in a genome, according to embodiments disclosed herein.

FIGS. 11A-11B illustrate exemplary embodiments of a non-coding RNA map, according to embodiments disclosed herein.

FIG. 12 illustrates a process for finding a variable and a non-variable sequence signature of a protein, according to some embodiments.

FIG. 13 is a flowchart illustrating steps in a method for identifying and displaying a cryptic site in a nucleotide string, according to some embodiments.

FIG. 14 is a flowchart illustrating steps in a method for creating and displaying a protein signature in an amino acid string, according to some embodiments.

FIG. 15 is a flowchart illustrating steps in a method for identifying and displaying a cryptic promoter site in a nucleotide string, according to some embodiments.

FIG. 16 is a flowchart illustrating steps in a method for identifying and displaying a cryptic poly-A site in a nucleotide string, according to some embodiments.

FIG. 17 is a block diagram illustrating an example computer system with which the client and server of FIGS. 1 and 2 and the methods of FIGS. 13-16 can be implemented.

In the figures, elements or steps having the same or similar labels are associated with features or processes having the same or similar description, unless otherwise stated.

### DETAILED DESCRIPTION OF THE DRAWINGS

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

The present disclosure is directed to a platform for the comprehensive analysis of gene regulation and splicing in genes within the human genome. The platform provides a basis for the analysis of regulatory and splicing elements in the human genome, their cryptic versions, and extensive details of the molecular processes that occur at every level of gene expression, transcription, splicing, and translation of every gene in the human genome. In some embodiments, the platform disclosed herein facilitates the analysis of mutations and aberrations in these processes at the structural, molecular, and sequence levels, and their associations with various diseases. A platform as disclosed herein enables the analysis of sequence elements and protein factors that assist in tissue specific gene expression and alternative splicing of transcripts, thus enabling the understanding of basic biological processes, and the mutations that cause various tissue and organ specific cancers and diseases. Further, it finds the potential additional genes in the unexplored region of the genome (e.g., the dark matter genome), and focuses on the analysis of the regulatory and splicing elements and their cryptic versions in these genes, and the mutations that occur in various diseases thereof. A platform as disclosed herein thus enables the thorough analysis of the processes of gene regulation and splicing, and the aberrations within them that cause diseases from genes in the human genome. This platform is useful to biologists, practicing clinicians, and clinical researchers to study and understand gene regulation and splicing and their aberrations.

Biologists have traditionally expected that most disease-causing mutations occur in protein-coding regions (CDS), as they directly affect the proteins. Thus, studies focused on regulatory elements have been largely ignored, and consolidated tools to address these regions have been lacking. However, it is increasingly becoming apparent that mutations in regulatory and splicing elements are responsible for upwards of 60% of all diseases. Embodiments as disclosed herein address this shortcoming of current genome analysis and provide a tool for a systematic analysis of the regulatory and splicing elements, and the effect of mutations in them. The platform provides the ability for a comprehensive analysis of gene regulation and splicing, and their aberrations due to mutations.

Human genes contain exons that are the protein-coding portions of the gene, and introns that do not code for the protein. The exons are sequence portions that are expressed into protein sequences, and the introns are the sequences that interrupt the exons and have regulatory sequences within them. Exons are usually short with an average length of ˜120 bases, whereas introns are usually very long, with an average length of 6,000 bases. The human genes contain an average of ˜10 exons, but a considerable fraction of genes consists of a large number of exons and introns, up to 200 exons. The gene is copied into an RNA transcript, from which the introns are excised and the exons are “glued” together to synthesize a functional protein.

The exons are spliced together to form the complete coding sequence, and the introns that interrupt the coding sequence are eliminated. A complex machinery called spliceosome, that contains ˜300 proteins and five small nuclear RNAs (snRNAs), carries out this splicing process. In addition to the coding sequence, there exists several regulatory elements including the promoter, transcription initiation site, splicing sites, branch point sites, enhancers and silencers of the promoter and splicing sites, poly-A addition sites, and un-translated regions upstream (5′ UTR) and downstream (3′ UTR) of the coding sequence.

Promoter sites regulate the expression of genes by binding the RNA polymerase enzyme to the promoter sequence(s) and initiating the transcription at the transcription start site. Several transcriptional regulatory proteins also bind to multiple regions of the promoter site and enable complex regulation of genes. Furthermore, elements known as enhancers and silencers of the promoter sites enhance or suppress gene expression respectively. By utilizing different promoter enhancers and silencers in various tissues and organs, the expression of tissue/organ specific genes are regulated. Mutations in any of these regions can disrupt gene regulation, thus leading to disease. In addition, mutations in the transcription initiation sites also affect the gene expression and can lead to disease.

RNA splicing is carried out using sequence signals bordering the exon-intron junctions. A splice sequence at the 5′ end of the intron (e.g., the donor site), and another splice sequence at the 3′ end of the intron (e.g., the acceptor site), aids in this process. In addition, a signal known as the branch point sequence within the intron also assists in the process of splicing. Mutations in these regions lead to aberrations in the splicing process, and are known to cause numerous cancers and non-cancer diseases. Enhancers and silencers of these splice sites are present within the exonic and intronic regions, which enhance or suppress the splicing process, respectively. By utilizing different splicing enhancers and silencers in various tissues and organs, the spliceosome can produce alternative splicing transcripts in different organs. Mutations in these regions also lead to diseases.

The 5′ un-translated region (5′-UTR) of an mRNA plays a critical role in the regulation of translation. It contains functional elements that fine-tune the process of protein expression. Mutations in these regions are associated with a number of human diseases. The poly-A addition sites present in the 3′ UTR region contribute to mRNA stability, translation control, and nuclear export of the mRNA. There may exist multiple poly-A sites in many genes, thus producing more than one transcript from a single gene. Mutations in these sites lead to disruption of these processes causing numerous diseases.

For every regulatory element, there exists sequences that resemble the genuine (real) sequences within the gene and are known as cryptic sites. When mutations occur in the real or cryptic sites, cryptic sites may be used instead of the real sites and may cause aberrations in the transcription, splicing, and translation processes. Thus, it is desirable to identify and analyze cryptic regulatory and splicing elements and cryptic exons. Cryptic regulatory elements and cryptic exons play a major role in disease causation. Analysis of these sites paves the way to identify disease associations, diagnosis, and treatment of several diseases.

There are thus multiple regulatory elements and their cryptic versions in every gene that are desirable to correctly transcribe, splice, and translate the gene to its protein. Mutations in any of these regions may disrupt each of these processes leading to cancers and several other diseases. It is increasingly realized that errors in these processes contribute to disease. However, the field has traditionally focused on the coding regions of the genes for deciphering the genetic causes of diseases, largely ignoring the regulatory regions.

The coding regions of genes constitute only 2% of the human genome, and the other 98% are introns, which do not code for proteins. In addition, the known genes only include ˜30% of the genome. The remaining ˜70% of the genome are intergenic regions without any known genes. However, genes that are not yet discovered may occur in these regions, and mutations within them may cause diseases. In addition, genes can also occur within the long intron sequences in the known genes. A platform as disclosed herein identifies these genes in unexplored regions of the human genome (e.g., the dark matter genome), and applies its features on these genes to further study the regulatory and splicing processes and disease-causing mutations in them.

Embodiments of this disclosure may analyze all or multiple portions of the human genome including non-coding (nc) RNA genes (e.g., tRNA, rRNA, miRNA, snoRNA, snRNA, and lncRNA), and further drilling down into the sequence views of elements displayed on the gene sequence, depicting them in different color codes. Embodiments of a platform as disclosed herein also provide statistics analyzed for selected genes in various organisms, other than human, including animals, microbes, plants, fungi, and viruses. A platform as disclosed herein may display the information on “before and after exon exclusion” with statistics of domains, total number of different consequences for all possible/predicted domains, and exon exclusion events.

Proteins have a paramount importance as the functional and structural unit of a number of physiological processes. An aberration in the protein structure may lead to molecular diseases with profound alterations in biological and metabolic functions. A protein contains one or more domains which are its basic units. Each domain carries out a specific biochemical or biological function, and using multiple domains together, a protein can accomplish complex biological functions. Although genes carry the biological information that constructs an organism, it is the proteins that are the true workhorses of the cells, tissues, and organs, and in fact, the whole organism. The sequence of a protein is not constant; rather, it is variable. There can be more than one amino acid present at many positions in a protein sequence, without altering the structure and function of the protein, which are called variable amino acid positions. Only at some positions, an amino acid is invariant or can vary to a limited number of amino acids, which are called invariant or low variant amino acid positions.

Accordingly, high amino acid variability occurs frequently in a protein, as only a few amino acids may be desirable in specific locations to enable key functions such as the active site of the protein. The rest of the amino acids aid to bring about the three dimensional structure of the protein in such a way that the active sites are correctly placed to carry out its function. These amino acids, other than the active sites, can be allowed to vary (e.g., be replaced by other amino acids), without altering the structure or function of a protein. Thus, protein sequences exhibit amino acid variability or degeneracy, which provides a definite set of variable amino acids at each position of the protein, forming a variable amino acid sequence signature. There exist few invariable positions, which exhibit one to few allowed amino acids. Mutations in these invariable or low variable positions will alter the active sites for the protein that lead to a defective protein unable to carry out its function, and thus lead to disease. Most of the mutations in the highly variable positions are tolerated and are said to be benign. In addition, an alternating rhythm of hydrophilic (water loving) and hydrophobic (water repelling) amino acids has been found to be largely sufficient to maintain the protein structure and function. Thus, the pattern of these hydrophobic and hydrophilic amino acids forms a major part of the study of protein structure and function, and the aberrations due to mutations. In addition, protein domains form secondary structures that have implications for protein stability, which are affected by mutations in disease. Accordingly, embodiments as disclosed herein provide a platform to visualize, analyze, query, and search for variability in protein structure and signature, and to correlate with it the effect (deleterious or not), in the phenotypic trait of a subject, of mutations or other genetic aberrations.

Embodiments as disclosed herein provide a robust platform for a comprehensive and thorough analysis of the genetic and associated molecular processes of disease and other phenotypic traits. It further enables the analysis of mutations and aberrations in these processes at the structural, molecular, and sequence levels, and their associations with various diseases and disorders. Thus, a platform consistent with the present disclosure provides a basic foundation for the analysis of regulatory elements in the human genome. This foundation enables further analysis of sequence elements and protein factors that assist in tissue specific gene expression and alternative splicing of transcripts, thus enabling the understanding of the basic biological processes and diseases.

Alternative splicing is a regulatory process occurring in eukaryotes, where it greatly increases the biodiversity of proteomes that can be encoded from the genome. During gene expression, exons are spliced alternatively in different isoforms that results in multiple proteins coded by a single gene. In this process, specific exons of a gene may be included or excluded from the processed messenger RNA (mRNA) produced from that gene. Consequently, the proteins translated from alternatively spliced mRNAs will contain differences in their amino acid sequence and, often, in their biological structure, functions, and clinical associations. The several modes of alternative splicing that are generally recognized are, exon skipping (one of the most common modes), in which an exon may be spliced out of the primary transcript or retained; mutually exclusive exons, either one of two exons, is retained in mRNAs after splicing; alternative donor site, an alternative donor other than that in the canonical transcript is used, changing the 3′ boundary of the upstream exon; alternative acceptor site, an alternative 3′ splice junction is used, changing the 5′ boundary of the downstream exon; intron retention, a partial intron sequence may be retained. Furthermore, there occur other mechanisms of generating different mRNAs from a single gene such as multiple promoters and multiple polyadenylation sites. Use of multiple promoters is properly described as a transcriptional regulation mechanism rather than alternative splicing; by starting transcription at different points, transcripts with different 5′-most exons can be generated. At the other end, multiple polyadenylation sites provide different 3′ end points for the transcript.

The production of alternatively spliced mRNAs is regulated by a system of trans-acting proteins that bind to cis-acting sites on the primary transcript, including splicing activators that enhance the usage of a particular splice site, and splicing repressors or silencers that reduce the usage of a particular site. Mechanisms of alternative splicing are highly variable and various methods are used to elucidate and predict the regulatory systems involved in splicing by a “splicing code.” In addition, errors in alternative splicing due to mutations in the splice sites, cryptic sites, and branch point sites, enhancers and silencers, and other regulatory elements can lead to aberrations in alternative splicing resulting in truncated or defective protein. Splicing aberrations have a profound impact contributing to a larger proportion of genetic disorders, various cancers, and other diseases.

Embodiments as disclosed herein provide a computer-implemented platform to address both of these mechanisms with an alternative splicing module to query and analyze a variety of mRNAs that may be derived from a gene. In some embodiments, the alternative splicing module provides analysis of potentially deleterious effects of alternative splicing aberrations, and a correlation of these with disease and other phenotypic traits in a subject.

The current understanding of molecular biology is that the flow of information takes place from DNA, to RNA, to protein through various biological processes. The first step is the formation of the RNA transcript (copy) of the gene that forms the bridge between DNA to the mature mRNA that is ready to be translated into proteins. Upstream of the gene sequence lies the promoter sequence, which forms the control region that switches on or off the gene. The RNA polymerase binds to this promoter sequence to make the RNA transcript. The introns in the RNA transcript are spliced out thereby linking together the exons to make the mature mRNA. There exist regulatory sequences upstream and downstream of the mRNA region within the transcripts that are not translated into protein. These un-translated regions, known as the 5′ UTR (upstream) and 3′ UTR (downstream) contain regulatory elements that regulate the transport and translation of the mRNA into protein.

Accordingly, embodiments consistent with the present disclosure illustrate the properties of these promoters and un-translated regions (UTRs) in the gene transcripts and mRNA sequences, enabling further interactive analysis by the user. Some embodiments include classifying exons in the gene according to whether they are coding, partially-coding, or non-coding, and shows splice site sequences and their scores. It then locates any upstream and downstream open reading frames (u-ORFs and d-ORFs) that surround the real ORF of the mRNA. A Kozak consensus sequence is a motif that functions as the protein translation initiation site within the mRNA. A mutated, wrong start site can result in non-functional proteins and have implications in human diseases. In some embodiments, a platform as disclosed herein calculates a score for Kozak consensus sequences that exist upstream and downstream of the start codon ATG, which would indicate which of the u-ORFs and d-ORFs may be turned on in different biological contexts.

A branch point sequence is a regulatory element that aids the spliceosome to form a loop with an intron before splicing an upstream exon with a downstream exon to form the mRNA. Embodiments as disclosed herein enable the analysis of branch point sequences and their cryptic versions to play an important role in understanding the molecular mechanisms of splicing and their disease associations.

Mutations within branch point sequences disrupt the lariat formation and result in aberrations in splicing. Incorrect splicing due to aberrations in branch point sequences are responsible for 9-10% of the genetic diseases that are caused by point mutations and lead to various effects in splicing, including exon skipping due to improper binding of the SF1 and U2 snRNP splicing proteins and disruption of the natural acceptor splicing site or intron retention (whole or its fragment) if they create a new 3′ splice site. Mutations within a cryptic branch sequence may cause aberrations that can incorrectly splice the gene transcript and lead to various cancers and other diseases. Accordingly, a platform as disclosed herein identifies real and cryptic branch point sequences throughout the genes and possible mutation events thereof. Moreover, embodiments as disclosed herein may correlate the findings with publicly available disease and annotation databases.

A platform as disclosed herein may determine whether a mutation in the branch point sequence may cause splicing aberrations and the type and mechanism of such aberrations (such as exon skipping and intron inclusion). Thus, the platform can be ideal to discover novel branch point mutations from the individual subject's genome or genomes from a cohort of subjects. The platform's approach of predicting real and cryptic BPS within a gene or any sequence, and detecting the deleterious mutations within the branch points acts as an effective strategy for clinicians and researchers in analyzing the splicing defects associated with disease. Also, branch point mutations establish a valuable resource for further investigations into the genetic encoding of splicing patterns and interpreting the impact of common and disease-causing human genetic variation in gene splicing.

Non-coding RNAs (ncRNAs) are functional molecules that are only transcribed and not translated into proteins. A large fraction of the human genome constitutes non-coding elements such as small non-coding RNAs (miRNA, piRNA, SiRNA, SnRNA), and long non-coding RNAs (linc RNA, NAT, eRNA, circ RNA, ceRNAs, PROMPTS). These ncRNAs mediate the regulation of gene expression, and play critical roles in defining DNA methylation patterns. The mis-regulation of lncRNAs is often associated with cancer and other diseases.

Transfer ribonucleic acid (tRNA) helps in decoding the messenger RNA (mRNA) into a protein. tRNAs function at specific sites in the ribosome during the translation process, synthesizing a protein from an mRNA molecule. tRNA also has introns 14-60 bases in length that interrupt the anticodon loop. tRNA splicing is a rare form of splicing that involves a different biochemistry than the spliceosomal and self-splicing pathways.

Ribosomal RNA (rRNA) associates with a set of proteins to form ribosomes. These complex structures, which physically move along an mRNA molecule, catalyze the assembly of amino acids into protein chains. They also bind tRNAs and various accessory molecules necessary for protein synthesis.

MicroRNAs (miRNAs) are key regulators of biological processes in animals. These small RNAs form complex networks that regulate cell differentiation, development, and homeostasis. Deregulation of miRNA function is associated with many human diseases, including cancer. Thus, it has become important to understand the mechanisms that modulate miRNA activity, stability and cellular localization through alternative processing and maturation, sequence editing, post-translational modifications of Argonaute proteins, viral factors, transport from the cytoplasm, and regulation of miRNA-target interactions. In addition, analysis of mutations in miRNA genes are key to understanding the disease in subjects and in cohorts.

Cellular mechanisms controlling the gene expression by microRNAs and alternative splicing have an effect on proteome diversity and have been implicated in complex diseases such as cancer and other disorders. Variations in the miRNA sequence and/or variations in the miRNA target region of a transcript can have a major impact on post-transcriptional regulation. Events of alternative splicing can occur in more than half of the human genes, thereby changing the sequence of key proteins related to drug resistance, activation, and metabolism. Furthermore, alternative splicing and miRNAs can work together to differentially control genes.

Embodiments as disclosed herein illustrate molecular aberrations of variants in the ncRNA genes and their correlations with diseases including cancers, non-cancer diseases, and multisystemic disorders. The mutations that disrupt the cellular functions which are dependent on non-coding RNA genes, or the factors required for the RNA functions, can be deleterious. The tRNA, rRNA, miRNA, siRNA, snoRNA, snRNA, and lncRNA genes are analyzed and the pathogenicity of mutations within them are established which is of major diagnostic importance. Approaches are explored to modify the splicing pattern of a mutant ncRNA or replace an RNA gene that bears a disease-causing mutation to achieve therapy.

Embodiments as disclosed herein identify and illustrate SNPs and Indels at the miRNA-related functional regions such as 3′-UTRs and pre-miRNAs and are key targets to uncover gene dysregulation resulting in susceptibility to or onset of human diseases. The deleterious mutations in the mitochondrial transfer RNA (mt-tRNA) and mitochondrially encoded rRNA (mt-rRNA) genes are known to cause many genetic diseases. Defects in oxidative phosphorylation in mitochondria are often associated with impairment of processes such as replication, transcription, or translation of mtDNA, which can be due to mutations in either of the mtDNA-encoded RNAs (tRNAs and rRNAs). mt-tRNA mutations can lead to several diseases including neurosensory non-syndromic hearing loss, diabetes mellitus, and a diverse range of clinical phenotypes. si-RNA mutations also may be involved in disease. Discovering the disease-causing mutations in the ncRNA genes, and identifying the molecular mechanisms of disease, has the potential benefit for both diagnostics and treatment of several diseases.

Moreover, embodiments as disclosed herein enable the identification of the various ncRNA genes in a genome, and the details of these genes including their promoters, exons, introns, and their associated enhancer/silencer elements, prediction of deleterious mutations and the molecular mechanisms, illustration of these details in gene structure, tabular and sequence views, and enabling the various interactive analysis capabilities. Furthermore, the analysis of variability in the ncRNA gene sequences plays an important role in deciphering the disease associations.

Other elements in embodiments as disclosed herein may include:

Exon Splice: to predict whether potential exon skipping events that arise through alternative splicing would maintain or destroy the open reading frame of the gene.

Cryptic Splice: to find cryptic splice sites and cryptic exons in each gene based on user-defined score thresholds.

Exon Chart: to generate a graph of exon lengths within each gene, creating a visual chart of patterns such as outlying exons and length repetition.

Alternative Splice: to depict alternative splicing events such as exon skipping, intron retention, and alternative splice site usage in each of the predicted isoforms of a given gene.

Exon Frame: to create an exon-intron map for each gene. It locates the exons in three reading frames and displays the patterns of stop codons within introns.

Protein Signature: to highlight allowed and not-allowed AA substitutions at each position in protein domains, generating a unique AA signature for each domain.

UTR view: to illustrate the untranslated regions of mRNA sequences, including promoters, uORFs, dORFs, start and stop codon contexts, and poly-A signals.

Branch Points: to enable the study of branch points in genes, their involvement in splicing of exons and cryptic exons, and the consequences of mutations in them.

Enhancers and Silencers of gene regulation and splicing: A map to provide insights on the enhancers and silencers of regulatory and splicing elements in human genes, their association in gene regulation and splicing events, and the effects of their mutations in dysregulation of genes.

Non-coding RNA Genes: A map that facilitates visualizing the processes of splicing and mutations within the different non-coding (nc) RNA genes, and their implication in human diseases.

Dark Matter Genomics: A map that describes all of the coding, gene regulatory and splicing elements and their cryptic versions in the new genes identified within the introns of known genes and within the potentially undiscovered genes within the long intergenic regions.

Splice database: a database for the findings from each of the Splice Atlas maps, providing an integrated platform to analyze the regulatory and splicing elements of every gene from the genome in a single view.

The human genome includes ˜3.2 billion bases. However, only 1-2% of the human genome codes for proteins. The coding sequences (exons) for proteins constitute a very small fraction of the gene itself, and the rest of the gene consists of introns and un-translated regions. The introns in numerous genes are extremely long, often longer than 100,000 bases and up to more than a million bases, which may contain unknown genes and regulatory sequences. In addition, there exists large regions of DNA sequences located between genes, defined as intergenic spaces. The function of most of these regions are currently unknown. However, these regions may contain sequences that regulate nearby genes, long non-coding RNAs, and genes that are yet undiscovered. Together, these non-coding genomic regions, that include the introns in the currently known genes and the intergenic regions between the currently known genes, constitute ˜98% of the genome. It is thought that these regions defined as the dark matter of the genome may be very important to the functioning of the genome, and mutations in them may lead to numerous diseases.

Embodiments of the present disclosure define the dark matter genome as the regions within the genome that include the introns in the currently known genes and the intergenic regions between the currently known genes. Accordingly, a platform consistent with the present disclosure defines white matter genome as the currently known and annotated genes, excluding the potential genes present within the introns. In some embodiments, a platform as disclosed herein identifies potential genes, protein-coding sequences, and the regulatory regions of these protein-coding genes, as well as the non-coding RNA genes, in the dark matter genome. Accordingly, some embodiments applied the functionalities of multiple modules therein on these newly discovered genes and obtained the various details for CDS and regulatory genetic elements, and their cryptic versions that occur within these genes. Some embodiments include modules to focus on the dark matter of the genome to unravel their hidden wealth and enable the discovery of important genetic information that will advance the understanding of disease and drug response, ultimately benefiting the practice of medicine. It aims to decipher these important regions within the dark matter of the genome and discover their involvement in disease by uncovering them in cohort studies from subjects with different diseases and adverse reactions to different drugs.

Accordingly, some embodiments work on a basic principle that deleterious, disease-causing mutations would be enriched in the gene(s) that cause the disease in a cohort of subjects within any of the genetic elements including the CDS, and the different regulatory elements in the gene, such as the promoter, UTR, splice donor, acceptor, and branch sites, enhancers and silencers, and poly-A sites, and their cryptic versions throughout the gene sequence. Thus, the platform approaches the discovery of the disease-causing genes by identifying the deleterious mutations in multiple different regulatory elements and their cryptic versions throughout the gene across the subject cohort. It also approaches this problem by identifying the deleterious mutations from selected elements within the intergenic regions, as the cryptic versions of these elements occur throughout the genes including the other elements, UTR, exons, and introns, and the intergenic regions. Embodiments as disclosed herein use the Shapiro & Senapathy (S&S algorithm) method and other relevant algorithms for detecting the splice sites and mutations in them, to develop unique scoring methods for the different regulatory elements by using the unique PWMs for the different elements based on their respective consensus sequences and the specific lengths of these elements. With this basic approach, Splice Atlas has discovered that it is able to identify deleterious mutations enriched within the different regulatory regions in addition to the coding regions. Furthermore, it has discovered that the deleterious disease-causing mutations are enriched in cryptic sites for the different regulatory elements that occur throughout the genes.

The human genome is currently thought to consist of ˜19K genes (19,127). However, it is likely that genes in the human genome are not yet deciphered for several reasons. The gene finding programs rely on the knowledge of known proteins to determine if a gene should be considered valid. There are ˜300 types of tissues in human, and many of the proteins expressed in them are of very low frequency, which are yet unknown. Furthermore, there are a large number of genes that are activated at different space-times, and then switched off, during the embryological development, many of which are also unknown. Thus, many proteins are yet to be uncovered from the human genome, which may occur within the long introns (>10,000 bases, 20,521 introns in the human genome) and within the intergenic regions (total sequence of length 2.8 billion bases).

Dark Matter Genome

| Human Genome Length | 3.2 | billion bases |
|---|---|---|
| Number of genes | 19,127 | |
| Length of all genes | 1.2 | billion bases |
| Total number of all exons | 200,603 | |
| Total length of all exons | 62.5 | million bases |
| Total number of all introns | 181,458 | |
| Total length of all introns | 1.1 | billion bases |
| Total number of introns >10,000 bases | 20,521 | |
| Total length of introns >10,000 bases | 728 | million bases |
| Total length of all intergenic regions = Length | 2.0 | billion bases |
| of the genome − Total length of all genes | | |
| Total length of all Dark Matter Genome = Length | 3.09 | billion bases |
| of all introns from current genes + length of | | |
| all intergenic regions | | |
| Total length of introns >10,000 bases + total | 2.8 | billion bases |
| length of intergenic region | | |

Data for this table have been obtained from our analysis of the human genome data from the NCBI (GRCh37.p13 assembly).

The estimates of the number of genes in the human genome vary considerably from ˜20,000-40,000. The current estimate from the National Human Genome Research Institute is 30,000. In addition, the number of genes in the human genome was thought to be 24,500 until 2007. In that year, by tweaking the maximum ORF length a bit shorter, the number of genes reduced to ˜20,000 based on the lack of their evolutionary conservation. It is also reasonable to expect that the current limit in the number of human genes reflects a desire to enable a practical set or catalogue of genes for research and medical applications, although many more genes could exist. Thus, there are strong reasons to expect that there could be many more genes yet to be discovered in the human genome.

Embodiments as disclosed herein include methods to identify and explore these undiscovered genes. A platform as disclosed herein uses multiple gene finding software programs (including the Shapiro & Senapathy, Splice Atlas Splice Code, GenScan, Augustus, and GeneID) to find genes from the dark matter genome. In addition, a platform as disclosed herein uses the PfamScan database to uncover potential domains in these genes. These processes are expected to produce overlapping genes. However, they are advantageous to ensure that genes are not missed from the intergenic regions. Furthermore, a platform as disclosed herein could enable other platforms to use these newly discovered genes in individual subject, family, and cohort studies, wherein the occurrence of disease relevant mutations in these regions can be determined. Embodiments as disclosed herein also enable the application of all of its maps on the newly found genes from each of the gene finding programs, and creates a database of selected data. This further enables the analysis of subject mutations from dark matter genes to identify the known and subject mutations that cause disease and drug response phenotypes.

### Example System Architecture

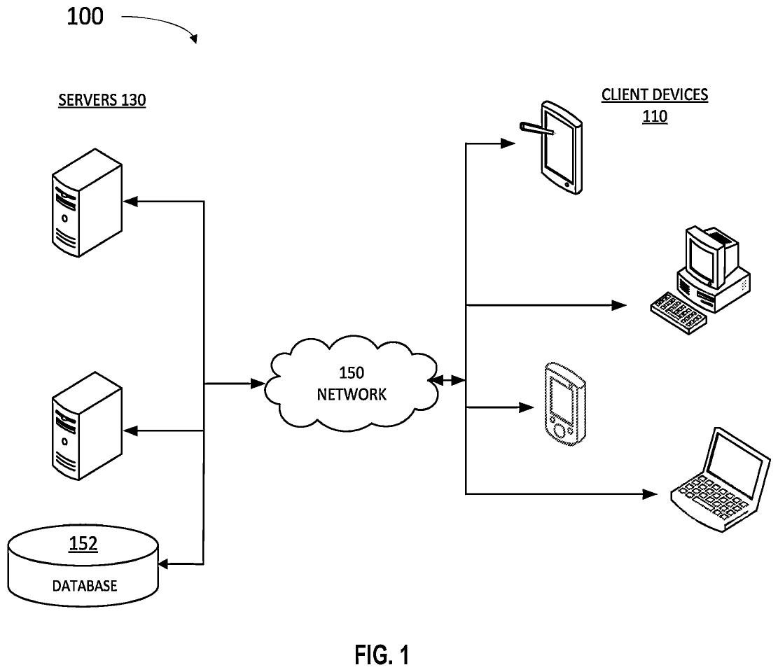

FIG. 1 illustrates an architecture 100 of devices and systems for providing a map of genes and mutations thereof for an individual or a cohort of individuals, according to some embodiments. A server 130 may be coupled with a database 152 storing a genome sequence log for each of multiple users handling client devices 110. Servers 130, database 152, and client devices 110 may be communicatively coupled with each other via a network 150.

Servers 130 may interact and communicate with other devices in network 150 via any one of multiple interfaces and communications protocols (e.g., wired, cable, wireless, and the like). More specifically, servers 130 and client devices 110 may include an appropriate processor, memory, and communications capability, configured to interact with network 150 via a digital interface. Client devices 110 may include, for example, desktop computers, mobile computers, tablet computers (e.g., including e-book readers), a digital stand in a retailer store, mobile devices (e.g., a smartphone or PDA), wearable devices (e.g., smart watch and the like), or any other devices having appropriate processor, memory, and communications capabilities for accessing one or more of servers 130 through network 150. In some embodiments, client devices 110 may include a Bluetooth radio or any other radio-frequency (RF) device for wireless access to network 150. The memory in the client device from the retailer may include instructions from an application programming interface (API) hosted by server 130 (e.g., downloaded from, updated by, and in communication with server 130). The API in client devices 110 may be configured to cause client devices 110 to execute steps consistent with methods disclosed herein.

Network 150 can include, for example, any one or more of a local area network (LAN), a wide area network (WAN), the Internet, and the like. Further, network 150 can include, but is not limited to, any one or more of the following network topologies, including a bus network, a star network, a ring network, a mesh network, a star-bus network, tree or hierarchical network, and the like.

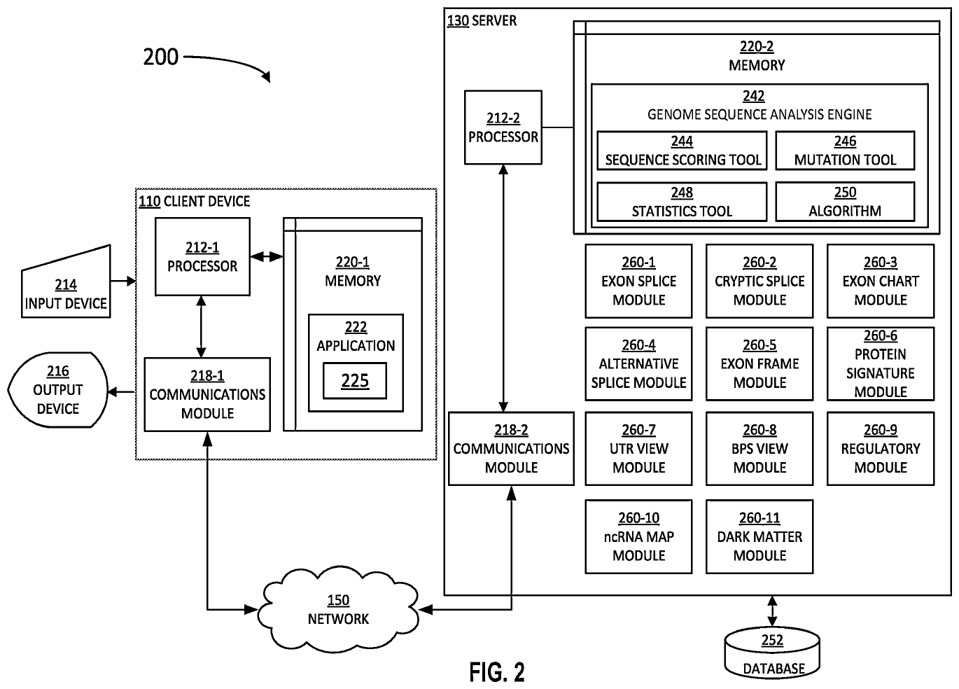

FIG. 2 is a block diagram 200 illustrating an example server 130 and client device 110 in architecture 100, according to certain aspects of the disclosure. Client device 110 and server 130 are communicatively coupled over network 150 via respective communications modules 218-1 and 218-2 (hereinafter, collectively referred to as “communications modules 218”). Communications modules 218 are configured to interface with network 150 to send and receive information, such as data, requests, responses, and commands to other devices on the network. Communications modules 218 can be, for example, modems or Ethernet cards. Client device 110 may be coupled with an input device 214 and with an output device 216. Input device 214 may include a keyboard, a mouse, a pointer, or even a touch-screen display that a user may use to interact with client device 110. Likewise, output device 216 may include a display and a speaker with which the user may retrieve results from client device 110. Client device 110 may also include a processor 212-1, configured to execute instructions stored in a memory 220-1, and to cause client device 110 to perform at least some of the steps in methods consistent with the present disclosure. Memory 220-1 may further include an application 222, including specific instructions which, when executed by processor 212-1, cause a graphic payload 225 hosted by server 130 to be displayed for the user in output device 216. Graphic payload 225 may include multiple graphic illustrations of a nucleotide string requested by the user to server 130. The user may store at least some of the illustrations and partial nucleotide strings from graphic payload 225 in memory 220-1.

In some embodiments, memory 220-1 may include an application 222, configured to display and process the contents in graphic payload 225. Application 222 may be installed in memory 220-1 by server 130, together with the installation of an operating system that controls all hardware operations of client device 110.

Server 130 includes a memory 220-2, a processor 212-2, and communications module 218-2. Processor 212-2 is configured to execute instructions, such as instructions physically coded into processor 212-2, instructions received from software in memory 220-2, or a combination of both. Memory 220-2 includes a genome sequence analysis engine 242. In some embodiments, genome sequence analysis engine 242 includes a sequence scoring tool 244, a mutation tool 246, a statistics tool 248, and an algorithm 250 to manipulate genome sequence data and create charts and reports for graphic payload 225.

Sequence scoring tool 244 parses at least a portion of a nucleotide string from a genome to identify a splicing site therein. More specifically, sequence scoring tool 244 identifies, in a nucleotide string, at least two exons, at least one acceptor, at least one donor, and at least one intron between the at least two exons. In some embodiments, sequence scoring tool 244 may include identifying, in a nucleotide string, at least two exons, and at least one intron between the at least two exons, and a promoter sequence. In some embodiments, sequence scoring tool 244 may include identifying, in a nucleotide string, a poly-A addition site, wherein the poly-A addition site includes a poly-A site and a signal. In some embodiments, sequence scoring tool 244 may include identifying a first amino acid string corresponding to a functional protein or protein domain.

Mutation tool 246 may identify protein domains affected by mutations in the nucleotide string that may alter the splicing sites (according to sequence scoring tool 244). In some embodiments, mutation tool 246 may access a mutation log in database 252, to identify a recurring mutation over a cohort or a population of individuals. In some embodiments, mutation tool 246 may identify, in a nucleotide string, a positive signature when the nucleotide string codes an allowed amino acid in the functional protein, and a negative signature when the nucleotide string codes a non-allowed amino acid in the functional protein. In some embodiments, mutation tool 246 determines a deleterious effect of a mutation based on whether the mutation occurs within the positive signature or the negative signature in a protein domain. In some embodiments, mutation tool 246 identifies, in a nucleotide string coding a protein domain in the functional protein, a mutation leading to a disallowed amino acid. In some embodiments, mutation tool 246 determines a mutated hydropathy signature of the protein domain based on a hydropathy of a mutated amino acid. In some embodiments, mutation tool 246 determines a normal hydropathy signature of the protein domain based on a hydropathy of an allowed amino acid or a disallowed amino acid, and a deleteriousness score for the mutation based on a difference between the mutated hydropathy signature of the protein domain and the normal hydropathy signature of the protein domain. In some embodiments, mutation tool 246 also determines a deleteriousness score for the mutation based on whether a mutation occurs within a positive signature indicating no deleteriousness or a negative signature indicating a deleteriousness.

Statistics tool 248 may perform a frequency analysis over the splice sites and the mutations identified by sequence scoring tool 244 and mutation tool 246. In some embodiments, statistics tool 248 may use mutation logs and gene sequencing logs in database 252 to evaluate statistical data on a nucleotide string for an individual or a cohort of individuals, for analysis. Algorithm 250 may be a linear or non-linear algorithm, including a neural network, machine learning, or artificial intelligence algorithm used to identify and score splicing sites (e.g., for sequence scoring tool 244). For example, in some embodiments, algorithm 250 may include the Shapiro & Senapathy algorithm to score a nucleotide string as a splice site (e.g., a ‘donor’ site or an ‘acceptor’ site), a MaxEntScan algorithm, and an NNSplice algorithm, among others. Algorithm 250 may combine various algorithms including the updated version of the Shapiro & Senapathy algorithm to develop biological probability and impact of the various splicing event data throughout the genome.

In some embodiments, genome sequence analysis engine 242 enables a user to search a subject's genome based on the gene nomenclature, domain identifiers, clinical association, number of domains, and number of exons per domain based on the user's preferences. In some embodiments, genome sequence analysis engine 242 enables the user to search a subject's genome based on genes established and sourced from database 252 (e.g., a third party database such as NCBI). In some embodiments, genome sequence analysis engine 242 enables the user to search a subject's genome based on a protein domain identifier (e.g., using database 252 such as Pfam ID) according to a dropdown selection list in graphic payload 225. In some embodiments, genome sequence analysis engine 242 enables the user to search a subject's genome by a number of domains encoded by a gene. In genes with multiple domains, this search option is based on the genes with the highest number of domains. In some embodiments, genome sequence analysis engine 242 enables the user to search the subject's genome based on the number of exons within a gene. In genes with multiple domains, this search option is based on the domain that is encoded by the highest number of exons.

Database 252 blends NCBI, Ensembl, and UCSC to combat various needs of research and clinical genomics needed for the industry and handles complex queries and supplies seamless data towards various maps for splice. In some embodiments, database 252 is robust as it combines various external sources including NCBI's GRCh37.p13, monarch initiative, COSMIC—v91, ClinVar—clinvar_20200407, dbSNP—b153 and Pfam into one single database to handle various complex needs of splicing across genome. In some embodiments, database 252 collaborates GenBank, EMBL Data Library, DDBJ, NBRF PIR, Protein Research Foundation, SWISS-PROT, and Brookhaven Protein Data Bank into a unified data. In some embodiments, database 252 is scalable to adapt various organisms' data and incubated under optimum normalization mechanisms. So that collects and disseminates the burgeoning amount of nucleotide and amino acid sequence data. In some embodiments, database 252 includes high intense data, which reveals dark matter genomics with appropriate evidence material. In some embodiments, database 252 includes multi-level annotations including correlation between various variations and phenotypes, with supporting evidence. In some embodiments, database 252 includes an integrative database of abundance of several different types of coding and non-coding sequence of the whole genome. Database 252 provides data flexibility to generate or produce the high intensity data to determine real splice sites, cryptic splice sites, and cryptic exons from genes of the human genome.

In some embodiments, genome sequence analysis engine 242 enables the user to search genes having a high frequency of cryptic splice sites that can be searched based on the number of cryptic sites (e.g., ranging from 1-80,000). In some embodiments, genome sequence analysis engine 242 enables the user to search genes having a high frequency of cryptic exons (e.g., ranging from 1-100,000) in a transcript. The cryptic exons can be visualized for individual transcripts for the selected gene. In some embodiments, genome sequence analysis engine 242 enables the user to search genes with different ranges of cryptic scores that can be searched (for example, >50, >60, >70, >80, and >90 to choose from). The cryptic splice sites can be visualized for individual transcripts for the selected gene. In some embodiments, genome sequence analysis engine 242 enables the user to search genes with different ranges of cryptic exon scores (for example, >50, >60, >70, >80, and >90 to choose from). The cryptic exons can be visualized for individual transcripts for the selected gene. In some embodiments, in genome sequence analysis engine 242, the user can choose the canonical transcript with the most number of exons for viewing and analysis. In some embodiments, genome sequence analysis engine 242 enables the user to visualize the cryptic splice sites and cryptic exons for the genes falling under various exceptional features including an in-frame stop codon (TAA, TGA, TAG) inside the reading frame, that contains a seleno-cysteine stop codon (mostly TGA) in the coding sequence, or that contains no stop codons (TAA, TGA, TAG) at the end of CDS. In some embodiments, genome sequence analysis engine 242 enables the user to search the subject's genome based on the canonical and non-canonical transcript identifiers, which may be listed in graphic payload 225 for the user's selection. In some embodiments, genome sequence analysis engine 242 enables the user to search the subject's genome based on a clinical association. Accordingly, a disease association of somatic cancer, germline cancer, inherited disorders, industrial panels, drug metabolizing gene (DMG) panels, the American College of Medical Genetics and Genomics (ACMG) gene panels may be provided by genome sequence analysis engine 242 to the user in a dropdown list in graphic payload 225. Based on the selection criteria, genome sequence analysis engine 242 displays gene and transcript information for the user via graphic payload 225. For example, graphic payload may include a gene name, a chromosome number, a gene ID, a strand, a protein ID, a protein length, and a number of exons. In addition, graphic payload 225 may include an information strip including a “Gene Info” button to display details on gene ontology and phenotype.

Server 130 may also include different modules which, in collaboration with the tools in genome sequence analysis engine 242, enable the different applications and aspects disclosed herein. For example, some of the modules include an exon splice module 260-1, a cryptic splice module 260-2, an exon chart module 260-3, an alternative splice module 260-4, an exon frame module 260-5, a protein signature module 260-6, an “un-translated” (UTR) view module 260-7, a branch point sequence (BPS) view module 260-8, a regulatory module 260-9, a non-coding (nc) RNA map module 260-10, and a dark matter module 260-11 (hereinafter, collectively referred to as “modules 260”). Exon splice module 260-1 identifies exons in a nucleotide string, and provides data analysis regarding the proteins and protein domains codified by the exons, and the possible protein isoforms or deleterious effects produced by amino acid rearrangements and other effects or mutations.

Exon splice module 260-1 indicates exon splices and provides a prediction of exon splice consequences, and may include a visualization tool based on genes established and sourced from external databases, libraries, and sources. Exon splice module 260-1 may also include a search engine with a protein-wise nomenclature based on selected identifiers, in a drop-down selection list. Exon splice module 260-1 enables the search for genes based on various search criteria as mentioned above. Based on the selection criteria, the gene and transcript information are displayed. Information like gene name, chromosome number, gene ID, strand, protein ID, protein length, and number of exons are displayed along with details on gene ontology and phenotype on clicking the “Gene Info” button available in an information strip. In some embodiments, exon splice module is divided into three different sections: after splicing view, sequence view, and hydropathy. The consequences of exon splicing in the gene are depicted under the after splicing view tab, including AA maintained, AA changed, AA change+PTC, frameshift, frameshift+PTC, overlapping domains, domain disruption, and domain skipping. The complete coding sequence, before and after splicing, of the selected transcript are shown in the sequence view tab. The hydropathy plot depicts the hydropathy index values determined by various methods along the amino acids sequence of the selected transcript.

A list of genes from an external database, library, or resource (NCBL, ENSEMBL, and the like) may be downloaded and integrated into Splice database 252 to provide the list of genes, exons, coding sequence, 5′ and 3′ UTRs, poly-A signal sequences, promoter sequences, and clinical association of genes with diseases (as sourced from dbSNP, COSMIC, and ClinVar). The exons are classified based on their coding features into 5′ and 3′ noncoding sequences, 5′ and 3′ partially coding sequences, fully coding sequences, upstream open reading frames (uORFs), downstream open reading frames (dORFs), poly-adenylated tails, kozak sequence contents, and various promoter boxes (TATA, GC, CAAT, and initiator), each of which are computed, identified, and tagged.

Cryptic splice module 260-2 uses algorithm 250 (e.g., the Shapiro & Senapathy algorithm) to identify cryptic splice sites and cryptic exons in human genes. Cryptic splice module 260-2 is a beneficial tool that helps investigate splicing mutations in disease, as Cryptic Splice Sites (CSSs) and cryptic exons are known to be involved in numerous diseases. More generally, cryptic versions of every regulatory element occur within a gene sequence. Furthermore, cryptic exons also occur throughout the gene sequence. Cryptic splice module 260-2 identifies one or more of these elements throughout the gene sequence, and displays them in graphical, tabular, and sequence views. Cryptic splice module 260-2 also determines the mutations that occur within these elements, and displays the details in various forms of illustrations from a subject sequence data and from various public data sources including dbSNP, ClinVar, and COSMIC. Cryptic splice module 260-2 also identifies the cryptic versions of other regulatory elements throughout the gene sequence, and the mutations in them, and provides detailed illustrations in various forms.

Exon chart module 260-3 enables visual classification and analysis of exon lengths and their accompanying splicing features, including unusual exon patterns in distinct genes. In some embodiments, exon chart module 260-3 applies algorithm 250 (e.g., the Shapiro & Senapathy algorithm and other relevant algorithms) to determine the scores of real and cryptic splice sites in the outlier exons and other exons in a gene. In some embodiments, exon chart module 260-3 enables the analysis of outlying exons that have highly outlying lengths compared to the other exons in the gene, and their real splice sites, cryptic splice sites, real exons, cryptic exons, branch point sites, enhancers and silencers, and their scores. In some embodiments, exon chart module 260-3 displays regulatory elements and their cryptic versions within the outlying exon in graphical, tabular, and sequence views. In some embodiments, exon chart module 260-3 enables the graphical depiction of exons with repeated lengths and outlying exons in a gene, and their correlations with the splice donor, acceptor and exons scores, and their DNA and protein sequences, using dropdowns for user selection of these features and their involvement in disease. In some embodiments, exon chart module 260-3 enables various searching options using nested search boxes for the user to choose the genes with gene length, CDS length, genes having exon length repetition, exons with outlying lengths, disease associated with such genes, and exceptional genes with these features. In some embodiments, exon chart module 260-3 enables the search option for genes from various gene panels such as disease panels, drug metabolizing gene (DMG) panels, the American College of Medical Genetics and Genomics (ACMG) gene panels, and other user given gene panels and enabling the visualization and analysis of any gene provided. In some embodiments, exon chart module 260-3 provides the capability to analyze different exon classes based on length, length of the preceding and following exons and introns, and the scores of the acceptor and donor splice sites.

In some embodiments, exon chart module 260-3 provides the capability to analyze different sets of exons, each set with the same lengths, and their splice scores, exon sequences, amino acid sequences, and the ability to analyze various parameters such as if the sequences of exons of the same length are similar or different, and determining if the splice site sequences and scores are similar or different. In some embodiments, exon chart module 260-3 depicts the real and cryptic splice sites by employing Shapiro & Senapathy and other relevant algorithms and comparing the scores for exons with repeat lengths in genes from any given organism, including the human, in an automated manner. In some embodiments, exon chart module 260-3 enables the automated analyses of the many features of an exon chart and providing the tabular, graphical, and sequence representation for the analysis of every gene from any organism including animals, plants, and microorganisms. In some embodiments, exon chart module 260-3 classifies and analyzes exons based on their coding features into 5′ non-coding sequences, 3′ non-coding sequences, 5′ partially-coding sequences, 3′ partially-coding sequences, and fully coding sequences for the genes with repeated exon lengths and outlying exons. In some embodiments, exon chart module 260-3 characterizes the various exons present in a gene into multiple categories based on their length to identify the exon length repetition, highest exon lengths to signify the “outliers” in a gene, and the exception codons which contain no stop codon, in-frame stop codon, or selenocysteine codon sequences. In some embodiments, exon chart module 260-3 creates a repository containing information for genes in a genome such as exon details with the exon length, genomic position of the exons, transcript details, real/cryptic splice donors and acceptors, splicing scores, and enabling the display and analysis of any gene by a query. In some embodiments, exon chart module 260-3 enables a search for genes that fit various parameters of exon lengths, gene lengths, outlier exon lengths, exons with the same lengths, non-coding, partial coding and fully coding exon lengths, genes from different gene panels, and genes from different diseases, and determines if any disease correlates with such genes or vice versa, and the ability to analyze these genes in graphical, tabular, and sequence illustrations. In some embodiments, exon chart module 260-3 overlays the subject(s)' mutations on the gene with depictions in an exon chart, in graphical gene structure and sequence illustrations in color codes for depicting the features of exons, promoter boxes, 5′ and 3′ UTRs, real/cryptic splice sequences, poly-A site and region, branch point regions, and the ability to analyze them for different parameters of exons provided by an exon chart including the correlation of the subject mutations with gene features. In some embodiments, exon chart module 260-3 enables analysis of enhancers and silencers in the outlying exons, especially the first and last exons, to determine if the long lengths are required in order to accommodate these regulatory sequences or signals. In some embodiments, exon chart module 260-3 indicates the consequences of a mutation in graphical and sequence illustrations, and plotting subject mutations in a real or cryptic splice and exonic regions, and the known mutations from the different databases such as dbSNP, ClinVar, and COSMIC, and categorized into clinical significance, molecular consequence, variation type and pathogenicity based on the SIFT and/or PolyPhen scores on any gene chosen by the user. In some embodiments, exon chart module 260-3 enables the query and analysis of different parameters of genes in an exon chart for the detection and analyses of unusual length repetition patterns and splicing patterns in distinct genes, and possible disease connections.

In some embodiments, exon chart module 260-3 provides various information of exons in a gene and its associated elements such as protein family and domains, ontology information, disease phenotypes using i-icons, mouse hovers, and context-sensitive popups. In some embodiments, exon chart module 260-3 applies an exon chart algorithm and displays real and cryptic splicing elements, exons, introns, and abnormalities identified in the ncRNA genes (tRNA, rRNA, miRNA, snoRNA, snRNA, and lncRNA), and further drilling down into the gene structure and sequence views of elements displayed on the gene sequence, depicting them in different color codes. In some embodiments, exon chart module 260-3 enables the user-guide of the platform such as the “About” that provides context sensitive explanations for various features and applications, and “How To” that provides context sensitive information of how to use particular features throughout the different sections of the platform. In some embodiments, exon chart module 260-3 provides statistics for genes in a given organism and displaying the information based on different length ranges, comparing the distribution of genes having repetitive length, and genes having outlier exons, and depicting these statistics in various bar and pie charts. In some embodiments, exon chart module 260-3 enables the use of tightly coupled navigation by interlinking different sections to provide analysis of a gene, protein, or other elements and features throughout the platform.

Alternative splice module 260-4 uses algorithm 250 (e.g., the Shapiro & Senapathy algorithm and other relevant algorithms) to identify alternative splicing events such as exon skipping, intron retention, and alternative splice site usage in each of the predicted isoforms of the given gene. In some embodiments, alternative splice module 260-4 provides a catalog of predicted alternative transcripts in human genes, including those that may or may not genuinely encode distinct proteins. Alternative spice module 260-4 identifies unique splicing events in the alternative transcripts when compared with a canonical transcript, such as exon skipping, exon inclusion, intron retention, and alternative splice site usage.

Alternative splice module 260-4 maps alternative splice events and their molecular effects in different transcripts of a gene compared with the canonical transcript, which is defined by various methods. In addition, it also maps these details based on constitutive exons defined by various methods. In alternative splice module 260-4, differences among transcripts are also correlated with changes in the encoded structural domains, thereby capturing the functional regions of proteins that alternative splicing may normally or deleteriously affect. Alternative splice module 260-4 thus simplifies the prediction of the particular transcripts resulting in distinct proteins and distinguishes them with the artifacts of mistaken sequence annotation, which is key to the advancement of the field of clinical genomics and Precision Medicine. In some embodiments, alternative splice module 260-4 enables the visualization of known mutations, mutations from individual subjects and cohorts of subjects. In addition to the mutational analysis, alternative splice module 260-4 also provides analysis of the domains encoded by different isoforms of a gene in a single view. Thus, alternative splice module 260-4 provides insight into aspects of alternative splicing in genes, their impacts on functional domains, and mutational analysis.

Alternative splice module 260-4 provides multiple ways to view and analyze alternative splicing events, such as based on gene: The alternative splicing events can be visualized for individual transcripts for the selected gene; and based on clinical association: the alternative splicing events can be visualized for individual transcripts for the genes implicated in the panels for all major cancers and inherited disorders. In some embodiments, alternative splice module 260-4 provides alternative splicing events, wherein the user can select a particular transcript of a given gene and explore different alternative splicing events including skipped exons, cryptic exons, exons with alternative acceptor splice sites, exons with alternative donor splice sites, exons with alternative acceptor and donor splice sites, and retained introns together. In some embodiments, alternative splice module 260-4 identifies genes based on a number of transcripts (and selects the highest, or one of the highest): Genes having a high number of transcripts can be searched (e.g., ranging from 1-28). The alternative splicing events can be visualized for individual transcripts for these selected genes.

Alternative splice module 260-4 displays unique splicing events resulting in alternative transcripts when compared with the canonical transcript, such as exon skipping, exon addition, intron retention, and alternative splice site usage from any gene in a genome. In some embodiments, alternative splice module 260-4 correlates the differences among transcripts with changes in the encoded structural domains, thereby capturing the functional regions of proteins that alternative splicing may deleteriously affect, simplifies the prediction of transcripts ascertaining if the resulting amino acid sequences are distinct proteins or the artifacts of mistaken sequence annotation, and performs deeper pattern analysis of variations in alternative transcripts and alternatively spliced sites among different transcripts of a given gene. In some embodiments, alternative splice module 260-4 provides a “View All” option that layers the information from the canonical transcripts, current transcript, and the splice events across various methods such as “Canonical based” and “Exon based,” in one unified view. In some embodiments, alternative splice module 260-4 enables a user driven approach to identify and correlate the clinical association of mutations in various alternative splicing events including constitutive, cryptic, altered donor, altered acceptor, altered acceptor+donor, skipped exon, intron retention, in any cancers and non-cancer disorders. In some embodiments, alternative splice module 260-4 provides a pop-up window for explaining the alternative splicing events based on the occurrences of alternative spliced exons in different transcripts defined by various methods including canonical and constitutive, and displays the layers of events such as coding exons, pre-spliced domains, and splice events in the canonical transcript and transcript isoforms, and plotting the mutations from the subject and known mutations from various mutation-disease databases and highlighting them in both gene structure, tabular and sequence view, providing deeper analytical capabilities. In some embodiments, alternative splice module 260-4 displays possible alternative splicing events of the transcript in multiple views, based on possible combinations of canonical transcript and constitutive exons, including partially-coding and non-coding exons and un-translated regions. In some embodiments, alternative splice module 260-4 illustrates various forms of splicing events in a transcript having longest CDS, longest mRNA, highest number of mRNA exons, and highest number of coding exons, and depicting various forms of transcripts based on protein-coding and non-coding exons in the genes from the genome of any organism in an automated manner; and identifies mutations in the alternative splicing sites within the introns, exons, or any part of a gene from a subject, computing the scores for them using Shapiro & Senapathy and other relevant algorithms, and determining the pathogenicity of these mutations by comparing the scores with the splicing sites of normal sequence.

In some embodiments, alternative splice module 260-4 displays the locations of alternative splicing events and alternative spliced exons in a transcript and overlaying the subject(s)' mutations and known mutations from various public databases in graphical, tabular, and sequence view with pop-up boxes, mouse hovers, and context sensitive explanations. In some embodiments, alternative splice module 260-4 enables nested search boxes for the user to choose the genes based on number of transcripts, alternative splicing events (e.g., skipped exons, cryptic exons, exons with alternative acceptor splice sites, exons with alternative donor splice sites, exons with alternative acceptor and donor splice sites, and retained introns), disease associated genes, and exceptional genes; and predicts the splice events in genes from a portion of the genome or the whole genome without manual intervention, and enabling the automated analysis of every data point. In some embodiments, alternative splice module 260-4 provides information about the gene and its associated elements such as protein family and domains, ontology information, disease phenotypes using i-icons, mouse hovers, and context-sensitive popups while depicting alternatively spliced events and transcripts; and compares the domains within the selected transcript with those in the canonical transcript, and highlighting the portions of the canonical domains that have been removed, and the portions that have been added, in the selected transcript, in different colors.

In some embodiments, alternative splice module 260-4 displays the exon-intron structure of a selected gene with alternative splicing events in color-coded visuals and providing automated display of graphical and sequence illustrations in an expanded view; enables the search option for genes from various gene panels such as disease panels, drug metabolizing gene (DMG) panels, the American College of Medical Genetics and Genomics (ACMG) gene panels, and other user given gene panels; displays splice event details in an expanded view on clicking any of the splice events on the graphical illustration. In some embodiments, alternative splice module 260-4 includes an alternative splice prediction, analysis, and illustration to non-coding RNA genes (e.g., tRNA, rRNA, miRNA, snoRNA, siRNA); provides statistics analysis for genes in a genome, displaying the information on coding and non-coding sequences, distribution of alternative exon classes for each gene, and the frequencies of coding and non-coding transcript per gene, in tabular and graphical illustrations.

In some embodiments, alternative splice module 260-4 represents the consequences of a mutation in the alternatively spliced structures of the transcripts and the gene with graphical and sequence illustrations, and plotting subject mutations in a real or cryptic splice and exonic regions, and the known mutations from different databases such as dbSNP, ClinVar, and COSMIC, and categorized into clinical significance, molecular consequence, variation type, and pathogenicity based on the SIFT and/or PolyPhen scores, in graphical, tabular, and sequence illustrations. In some embodiments, alternative splice module 260-4 is applicable to any organism including animals, plants, and microorganisms. In some embodiments, alternative splice module 260-4 analyzes subject mutations overlaid on the genes to correlate their involvement in disease; analyzes mutations from databases such as ClinVar, dbSNP, and COSMIC on the genes to correlate their involvement in disease; and enables the user-guide of the platform such as the “About” that automatically provides context sensitive explanations for various features and applications, and “How To” that automatically provides context sensitive information of how to use particular features throughout the different sections of the platform. In some embodiments, alternative splice module 260-4 provides a map for genes in various organisms and displaying various statistics and information across the genome, enables the use of tightly coupled navigation by interlinking different sections to provide analysis of a gene, protein, or other elements and features throughout the platform; and enables an expanded version for each of the features of AltSplice map, which allows users to visualize and analyze further details in graphical, tabular, and sequence illustrations.

Exon frame module 260-5 determines the possible distribution of stop codons and coding exons in a reading frame before and after splicing events. A reading frame is a way of dividing the sequence of nucleotides into a set of consecutive, non-overlapping triplets, where these triplets equate to amino acids or stop signals during translation, which are called codons. In some embodiments, exon frame module 260-5 analyzes and verifies that a distance in the nucleotide string between two stop codons while mapping different stop codons should not fall inside an exon region. To verify this, the length of each of the exons and the open reading frame are plotted separately. The exon with maximum length in any transcript should be lesser than the maximum distance between two stop codons in all the reading frames. After splicing, the CDS length should be shorter than the maximum distance between two stop codons.

In some embodiments, exon frame module 260-5 allows the determination, analysis, and illustration of the exon-intron structures across ORF patterns of a gene and determines the structure of a gene with respective reading frames that contain exons of a gene and the patterns of before and after splicing by constructing an image of the entire split gene, including the exons, introns, splice junction signals, and stop codons that occur within each frame. In some embodiments, exon frame module 260-5 streamlines the detection of atypical gene patterns, such as long exons, long open reading frames without annotated exons, or short introns, and illustrates exons and ORFs in a single reading frame of the gene along with their splice sites and scores calculated using algorithm 250 (e.g., the Shapiro & Senapathy algorithm and other relevant algorithms). In some embodiments, exon frame module 260-5 represents three reading frames of a transcript, along with all possible stop codons in each reading frame and plotting the coding exons in appropriate reading frames by using the reading frame algorithm.

In some embodiments, exon frame module 260-5 displays exons, splice sites, branch sites, stop codons, and all possible splicing signals within UTRs, partially-coding and non-coding exons and un-translated regions in “Before” and “After” splicing visual illustrations, and provides capabilities for studying the distribution pattern of ORFs and exons in a gene by comparing their distribution and frequencies in the gene sequence and randomly generated sequences in graphics, tabular, and sequence views. In some embodiments, exon frame module 260-5 identifies mutations in a single reading frame from one or more subjects within the splice sites, exons, or any part of a gene, computing the scores for them using algorithm 250 (e.g., the Shapiro & Senapathy algorithm and other relevant algorithms), and determines the pathogenicity of these mutations by comparing the differences in the scores with the normal sequence. In some embodiments, exon frame module 260-5 illustrates mutations from the subject's genome and known mutations from various public gene-disease databases in graphical, tabular, and sequence view with pop-up boxes, mouse hovers, and context sensitive explanations, and displays a scatter plot showing the distribution of the lengths of ORFs, exons, spliced exons, mRNA, and CDS in the gene and randomly generated sequences, and enabling the comparison of these features between the gene and random sequences.

In some embodiments, exon frame module 260-5 enables nested search boxes for the user to choose the genes and transcripts based on exon and ORF length range, gene length, CDS length, disease associated genes, and exceptional genes, provides information about the gene and its associated elements such as protein family and domains, ontology information, disease phenotypes using i-icons, mouse hovers and context-sensitive popups, and enables search options for genes from various gene panels such as disease panels, drug metabolizing gene (DMG) panels, the American College of Medical Genetics and Genomics (ACMG) gene panels, and other user given gene panels. In some embodiments, exon frame module 260-5 displays a visual graphic of the structure of genes with exons, promoters, poly-A sites, stop codons, branch points, splice enhancers and silencers, and splice sites in a compact and expanded view along with relevant details showing the length of each exon, ORFs, and spliced exons in graphical, tabular, and sequence illustrations. In some embodiments, exon frame module 260-5 predicts the exon frame features of a gene, plotting the subject's mutations or any known mutations in the coding and non-coding regions, comparing the mutations from different gene-disease databases such as dbSNP, ClinVar, and COSMIC, determining the connection to various diseases, and visualizing their clinical impacts on gene structure and sequence illustrations, and represents the consequences of mutations that are categorized into clinical significance, molecular consequence, variation type, and pathogenicity based on the SIFT and/or PolyPhen scores.

In some embodiments, exon frame module 260-5 applies a single and three reading frames predictions, and displays the exons, introns, and abnormalities identified in the ncRNA genes (tRNA, rRNA, miRNA, snoRNA, snRNA, and lncRNA), and further analyzes the sequence analysis and visualizations of elements displayed on the gene sequence, depicting them in different color codes. In some embodiments, exon frame module 260-5 enables the user-guide of the platform such as the “About” that automatically provides context sensitive explanations for various features and applications, and “How To” that automatically provides context sensitive information of how to use particular features throughout the different sections of the platform. In some embodiments, exon frame module 260-5 determines and provides the ExonFrame statistics analyzed for all the genes in the human genome and various organisms, and displaying the information such as an average length of exons, introns, and ORFs, occurrences of stop codons in splice sites, codon distribution in splice sites, distribution of coding exon length, intron length, ORF length across the genome, and randomly generated sequence in tabular and graphical illustrations. In some embodiments, exon frame module 260-5 enables the use of tightly coupled navigation by interlinking different sections to provide analysis of a gene, protein, or other elements and features throughout the platform, illustrates patterns in expanded views, and shows more details in sequence views, and is applicable to various organisms including animals, plants, and microbes.

Protein signature module 260-6 enables the analysis of selected protein features in a genome, and their aberrations due to mutations that lead to diseases and other afflictions such as adverse drug reactions. It further enables the visualization and analysis of various details including the exon-domain signatures, cryptic splice sites, and the protein signature showing variable amino acids at each position of the domains that provides a deeper understanding of the allowed and non-allowed amino acids of the domains. When a gene is chosen in protein signature module 260-6, coding exons of the selected gene and transcript are displayed with their corresponding domains overlaid as colored lines. Mutations on these coding exons can be visualized by selecting the mutation toggle option. On clicking the domains above their coding exons, domain details, and various types of signatures such as 20 colors, Positive-Negative, Hydro, Cryptic splice, Alternative splicing and Whole protein signature, are displayed for further analysis. In some embodiments, protein signature module 260-6 performs or collects alignment results from a third party database (e.g., database 252, including the Pfam database), including a seed alignment and a full alignment. In some embodiments, a seed alignment includes a set of manually curated amino acids from the domain sequences from several genomes and thus tends to have a smaller number of amino acids than the full alignment. In some embodiments, a full alignment includes a set of amino acids produced from several genomes that are aligned using Hidden Markov models, and the like.

In some embodiments, protein signature module 260-6 determines, analyzes, and illustrates the protein sequence signatures of a protein and its domains, their associated features such as the hydropathy and splicing, and the clinical and biological impacts of genetic mutations. In some embodiments, protein signature platform 260-6 provides a protein chart to determine and illustrate the analysis of variable amino acids in protein-coding sequences under three different tabs: Protein Overview, Cryptic Splice Sites, and Variant density. In some embodiments, protein signature platform 260-6 converts the amino acid alignments from Pfam database into amino acid signatures of proteins and their domains, by identifying the variable amino acids and avoiding the redundant amino acids at each position, and by determining if an amino acid occurs at greater than a specific fraction (e.g., 50%) of the aligned positions, thus incorporating a unique algorithm. In some embodiments, protein signature platform 260-6 defines an algorithm that identifies the different non-redundant amino acids at each position and includes them as the variable or allowed amino acids at that position, taking into account any position with “.” or “−” in the alignment indicating a gap, whereby a position with a particular frequency (e.g., >50%) of dots is defined as grey regions in the signature.

In some embodiments, protein signature platform 260-6 determines and displays the set of non-redundant AAs produced from the multiple sequence alignment (MSA), generating a unique signature of allowed AAs for every sequence position, showing each of the 20 AAs in a distinct color, and defines that the allowed and non-allowed regions of the positive-negative signature of a domain or protein determines the pathogenicity or deleteriousness of a variant by its occurrence in the positive (green) or negative (red) region. In some embodiments, protein signature module 260-6 displays the non-redundant AAs from the multiple sequence alignment (for e.g., Pfam database) in one color (e.g., green), and all other AAs in another color (e.g., red), showing a map of allowed (positive) and non-allowed (negative) AA substitution space across the sequence, indicating variants that may result in a viable or defective protein. In some embodiments, protein signature platform 260-6 finds that the deleterious (pathogenic) mutations would fall within the negative region (red) and that the benign or likely pathogenic mutations would fall within the positive region (green), and applying this finding in testing and determining if a given variant is deleterious or not, determines the impact and clinical significance of the mutations based on the occurrence of the altered amino acids within the negative amino acid space or the positive amino acid space, thereby showing the amino acids where the actual mutations occur by color codes, and depicts the signature for the exon encoded domains in color codes based on a hydropathy scale. Protein signature platform 260-6 displays the hydrophobic AAs in shades of a particular color (e.g., red), and hydrophilic AAs shown in shades of another color (e.g., blue) to create a heat-map of hydropathy. In some embodiments, protein signature platform 260-6 determines the secondary structure map of the amino acid signature using standard values of secondary structure, and depicting them in different color codes thus creating a color-coded secondary structure signature, which will change due to genetic mutations from a subject or from gene-mutation databases such as ClinVar, dbSNP, and COSMIC. In some embodiments, protein signature module 260-6 defines the secondary structure map of the amino acid signature using standard values of secondary structure, depicting them in different color codes, thus creating a color-coded secondary structure signature and enabling its illustration against the domain signature for the analysis of secondary structures correlating with signatures and mutations in various amino acids. In some embodiments, protein signature module 260-6 enables the illustration, visualization, and analysis of mutations in the 3D structure of the domain along with the amino acid variability in the allowed or non-allowed set of amino acids and correlating and determining the effects of the mutations in the domain.

In some embodiments, protein signature module 260-6 represents the structure of coding exons in a gene by a shape such as an oval or rectangle, and overlaying the protein domains encoded by the exons, as available in Pfam database or predicted by PfamScan, or any other amino acid alignment databases, correlating the clinical association of mutations in the CDS with cancers and non-cancer disorders in a user-driven approach, displaying various details of domains encoded by the exons such as domain identifier (PfamId), class, start and end position of the transcript encoding the domain, and coding exons using i-icons, mouse hovers, and context-sensitive popups, depicts the variable amino acids in the key regions of human proteins, such as domains and deriving the set of “allowed” amino acids by generating the multiple sequence alignments of diverse genomes, creating a signature of potential amino acid substitutions across the domain, and classifying the signature under different tabs including: 20 colors, Positive Negative, Hydro, Cryptic Splice, Alternative Splicing, and whole protein signature, and illustrates the alignment of amino acids under two different tabs: Seed and Full, and depicting the alignment which contains a set of allowed/curated amino acids in the Seed tab and the alignment which contains the set of amino acids produced by Pfam using Hidden-Markov models in the Full tab. In some embodiments, protein signature module 260-6 computes and depicts the signature of potential amino acid substitutions across the domain in color codes based on the hydropathy (hydrophobic and hydrophilic) index, charge of amino acids, and determining its region and impact on the cryptic and alternative splicing sites, creates and depicts the impression of the known amino acid substitutions or subject(s) mutations that are likely to maintain the structure and function of a given protein region, and the mutations that are likely to destroy the structure and function of the protein thus leading to disease, depicts the exons that encode a domain by overlaying the domains on the corresponding positions of the codon and AA sequences, and various features of domains and proteins against the gene sequence, and enables the selection of different score thresholds to view any cryptic splice sites or cryptic exons that occur within the CDS of different exons in different color codes, thereby identifying the cryptic splice sites and cryptic exons within real exons, whose mutations can disrupt normal splicing leading to defective protein and disease.

In some embodiments, protein signature module 260-6 depicts the positions on the signature in which the human amino acid sequence has a gap, but other genomes have amino acids, shown as a dash in the human sequence, in different color codes, and indicating the positions at which lesser or higher than a specific fraction of amino acids occur with or without a gap (e.g., 50%) in the sequence signature. In some embodiments, protein signature module 260-6 provides toggle options to turn on the mutations to overlay known mutations on the signatures from different databases such as dbSNP, ClinVar, and COSMIC, categorized into clinical significance, molecular consequence, variation type, and pathogenicity based on the SIFT and/or PolyPhen scores, and enabling the illustration of the amino acids, cryptic sites, and its scores in graphical, tabular, and sequence with pop-up boxes, mouse hovers, and context sensitive explanations. In some embodiments, protein signature module 260-6 analyzes cryptic sites and exons within the coding sequence of a protein by determining and depicting the cryptic splice sites and cryptic exons, real splice sites and exon positions and their scores in various color codes and shapes, based on different score thresholds within the coding exon sequences in tabular, graphical, and sequence illustrations, analyzes the alternative splicing of the exons coding for the domains and providing the signatures for the added or skipped region of the exons coding for the domain, and enables the pattern analysis of variations in protein and domain sequence signatures for different transcripts of a given gene.

In some embodiments, protein signature module 260-6 displays the number of samples for each variant from the COSMIC database for each domain position, and depicts the positions of a specific variant in a color (e.g., red), and positions with more than one variant are depicted in different colors, for example, as follows: two variants->blue, three variants->green, four variants->yellow (named as variant density plot), predicts the splice sites in the genes of any organism using Shapiro & Senapathy and relevant algorithms in an automated manner. Predicting and assigning the score for cryptic exons based on the cryptic donor and acceptor splice site scores, and detecting which amino acid mutation would make the protein defective based on the mutations from one or more subjects within the protein signature, based on where the mutation falls within the positive or negative amino acid space, and determining which mutations are correctly identified and which are incorrectly identified. In some embodiments, protein signature module 260-6 overlays the subject(s) mutations on the gene, and provides visual and analytical illustrations of the mutations from the subject(s) and known mutations from various gene-mutation databases in graphical, tabular, and sequence views with pop-up boxes, mouse hovers, and context sensitive explanations, enables various search options using nested search boxes for the user to choose the genes based on the domain, number of domains in a gene, families, average AA substitutions, alignment type, disease associated genes, domains using Pfam Identifier, and exceptional genes, and provides various information about the gene and its associated elements such as protein family and domains, ontology information, disease phenotypes using i-icons, mouse hovers, and context-sensitive popups.

In some embodiments, protein signature module 260-6 creates a repository for the ProtSig platform containing information for genes in a genome such as exon details with the encoding domains, genomic position of the exons, transcript details, exon length, protein structure, real and cryptic splice sites and exons, and enabling the display and analysis of these features for any selected gene, enabling the search option for genes from various gene panels such as disease panels, drug metabolizing gene (DMG) panel, the American College of Medical Genetics and Genomics (ACMG) gene panel, and other user given gene panels and enabling the display and analysis of any gene by a query, and identifying structural regions and allowed nucleotide variations as signatures, and the mutations and disease relationships, in non-coding RNA genes (e.g., tRNA, rRNA, miRNA, snoRNA, siRNA).

In some embodiments, protein signature module 260-6 enables the user-guide of the platform such as the “About” that automatically provides context sensitive explanations for various features and applications, and “How To” that automatically provides context sensitive information of how to use particular features throughout the different sections of the platform, provides and analyzes statistics for genes in various organisms, and displays various statistics and information such as the number of unique domains from Pfam in multiple genomes, number of protein isoforms with different numbers of domains, average number of domains per protein across the proteome, average domain signature characteristics, average number of exons across the genome, and enables the use of tightly coupled navigation by interlinking different sections to provide analysis of a gene, protein, or other elements and features throughout the platform.

In some embodiments, protein signature module 260-6 represents the consequences of mutation in these structures of the transcripts and the gene with graphical, tabular, and sequence illustrations, and plotting subject mutations, and the known mutations from different databases such as dbSNP, ClinVar, and COSMIC, and categorized into clinical significance, molecular consequence, variation type, and pathogenicity based on the SIFT and/or PolyPhen scores, overlays the subject(s)' mutation(s) on the gene and protein structure and sequence on which the real and cryptic splice site and exon mutations are depicted, and determines the connection to various diseases, and enables an expanded version for each of the features of ProtSig, which allows users to visualize and analyze further details in graphical, tabular, and sequence illustrations. In some embodiments, protein signature module 260-6 automatically updates various data from different databases such as NCBI, ENSEMBL, and Pfam, and including other databases, and presents the latest information on different features such as genes, proteins, domains, mutations, and diseases, and is applicable for different organisms, including the human, other animals, microbial organisms, and plants.

UTR view module 260-7 identifies the various promoter elements, 5′ and 3′ UTRs, poly-A sites, and various possible ORFs such as u-ORFs and d-ORFs, their sub classifications within these based on the specific start and stop codons, and their disease connections.

In some embodiments, UTR view module 260-7 identifies genetic elements in various tabs for analyzing the properties of promoters and UTRs in transcripts and mRNAs such as: mRNA sequence, splice score and promoter, displays the structure of mRNA transcript of a gene and illustrating and enabling the analysis of the properties of un-translated regions (UTRs) in human mRNA sequences, and enables the classification of exons in the transcript into coding, partially-coding, or non-coding exons, providing splice site sequences, and scores for each of them.

In some embodiments, UTR view module 260-7 locates any upstream and downstream open reading frames (u-ORFs and d-ORFs) that surround the real ORF (CDS), enables the determination of the Kozak consensus sequences surrounding the start codon, and providing Kozak scores for the identified ORFs in upstream and downstream regions, indicating which ORFs may be turned on in different biological contexts, and depicts the structure and sequence of mRNAs and locates the sequence components such as coding sequence, 5′/3′ UTRs, Poly-A signals, initiator ATG codons, stop codons that are in-frame with one or more ATGs, upstream ORFs (u-ORFs) and downstream ORFs (d-ORFs), and displays four different classes of ORFs in upstream and downstream regions of every mRNA transcript of genes, in tabular, graphical, and sequence views.

In some embodiments, UTR view module 260-7 illustrates different ORF classes such as u-ORF, r-ORF (real open reading frame), and d-ORF between 5′ and 3′ region of coding exons and depicts the occurrences of start and stop codons on the gene's mRNA and for every ORF classes in a graphical, and sequence view, determines the ORF classes and tabulating the features of them such as ORF type, ORF position, Kozak sequence, Kozak score, stop codon sequence, real stop codon score, and 4-base stop codon score, and illustrating them in graphical and sequence view, displays the splice sites for all the exons in a transcript and computing scores using the Shapiro & Senapathy algorithm and other relevant algorithms, and calculating and displaying the exon scores by taking the average of the acceptor and donor scores, and defines different UTR and exon classes in a transcript, and categorizing them as fully coding exon (FCE), 5′ partially-coding exon (PCES), 3′ partially-coding exon (PCE3), 5′ and 3′ partially-coding exon (PCE53), 5′ non-coding exon (NCES), and 3′ non-coding exon (NCE3).

In some embodiments, UTR view module 260-7 identifies the promoter boxes such as TATA, CAAT, GC, and transcription initiators in the gene by computing the scores with varying thresholds by adapting the Shapiro & Senapathy and other relevant algorithms for each of the identified promoter boxes, enabling toggle options to visualize the various promoter boxes in graphical illustrations of gene structure and sequence. In some embodiments, UTR view module 260-7 predicts the clinical consequences of the subject's mutations in promoter boxes and poly-A sites, and UTR elements in the gene graphically and in sequence illustrations, determining their pathogenicity based on mutated scores, correlating with disease, and conducts similar analyses for the known mutations from the different disease-gene databases such as dbSNP, ClinVar, and COSMIC, and categorized into clinical significance, molecular consequence, variation type, and pathogenicity based on the SIFT and/or PolyPhen scores. In some embodiments, UTR view module 260-7 computes the strong poly-A signals and depicts them on the gene and mRNA in tabular, graphical, and sequence illustrations, and the disruptive mutations based on the scores, uses the Shapiro & Senapathy algorithm in the identification of different elements of the promoter (boxes), poly-A sites, and UTR classes for a gene, and their cryptic versions, and identifies real and cryptic promoter and poly-A motifs and elements by adapting and modifying other relevant algorithms such as MaxEntScan, NNSplice, and Human splicing Finder throughout the gene sequence and genes in the genome and its application to subject and cohort genomics.

In some embodiments, UTR view module 260-7 identifies real and cryptic promoters and poly-A motifs and elements by adapting and modifying other relevant algorithms such as MaxEntScan, NNSplice, and Human splicing Finder throughout the gene sequence and genes in the genome. In some embodiments, UTR view module 260-7 identifies real and cryptic splice sites using promoter and poly-A motifs and elements by adapting and modifying other relevant algorithms such as MaxEntScan, NNSplice, and Human splicing Finder throughout the gene sequences and genes in the genome and identifying the known mutations from databases such as ClinVar, dbSNP, and COSMIC. In some embodiments, UTR view module 260-7 identifies real and cryptic promoter and poly-A motifs and elements by adapting and modifying other relevant algorithms such as MaxEntScan, NNSplice, and Human splicing Finder throughout the gene sequences and genes in the genome and identifying the mutations from subjects' genome. In some embodiments, UTR view module 260-7 enables various search options using nested search boxes for the user to choose the genes based on number of ORFs, number of promoter boxes, promoter box score, poly-A boxes, poly-A box score, exon classes, disease associated genes, exceptional genes, and other parameters.

In some embodiments, UTR view module 260-7 provides various information for elements such as exons, mRNA elements, promoter elements, and UTR elements in a gene and their associated elements such as protein family and domains, ontology information, disease phenotypes using i-icons, mouse hovers, and context-sensitive popups. In some embodiments, UTR view module 260-7 enables the search option for genes from various gene panels such as disease panels, drug metabolizing gene (DMG) panels, the American College of Medical Genetics and Genomics (ACMG) gene panels, and other user given gene panels and enabling the display and analysis of these genes on UTR view platform. In some embodiments, UTR view module 260-7 identifies and illustrates the exceptional gene exons with rare behaviors such as an in-frame stop codon, selenocysteine codon, or no stop codons present in the end of the CDS, and applies to non-coding RNA genes (e.g., tRNA, rRNA, miRNA, snoRNA, siRNA, lncRNA).

In some embodiments, UTR view module 260-7 enables the user-guide of the platform such as the “About” that automatically provides context sensitive explanations for various features and applications, and “How To” that automatically provides context sensitive information of how to use particular features throughout the different sections of the platform. In some embodiments, UTR view module 260-7 provides the UTR view statistics analyzed for all the genes in various organisms and displays the information on frequency of different elements such as promoter boxes, poly-A sites, and exons contained in coding and non-coding regions, several different classes of ORFs (u-ORFs and d-ORFs), average Kozak and 4-base stop codon scores from the different ORF classes, and distribution of real and false 4-base stop codons. In some embodiments, UTR view module 260-7 enables the use of tightly coupled navigation by interlinking different sections to provide analysis of a gene, protein, or other elements and features throughout the UTR view platform, updates latest data and information pertaining to the elements described in UTR view with increasing data sources. In some embodiments, UTR view module 260-7 applies to all organisms including human, other animals, plants, and microbial organisms, enables the depiction of cryptic splice sites on the 5′ and 3′ UTR regions using the Shapiro & Senapathy and other relevant algorithms, and analyzes subject mutations and known mutations from databases such as ClinVar, dbSNP, and COSMIC, overlaid on the genes to correlate their involvement in disease.

In some embodiments, UTR view module 260-7 identifies new promoter motifs and elements based on PWM methods, sliding window methods, motif search methods, methods using motif sequence lookups, and sequence alignment methods in long sequences up to more than 10,000 bases upstream of gene start. In some embodiments, UTR view module 260-7 identifies motifs and elements that are target(s) of sequence specific promoter binding proteins and genes (such as TP53, OBSCN, TAF3, and FAT3) based on PWM methods, sliding window methods, motif search methods, methods using motif sequence lookups, and sequence alignment methods in long sequences up to more than 10,000 bases upstream of gene start. In some embodiments, UTR view module 260-7 identifies new gene control motifs and elements including promoter silencers and enhancers, based on PWM methods, sliding window methods, motif search methods, methods using motif sequence lookups, and sequence alignment methods in long sequences up to more than 10,000 bases upstream of gene start. In some embodiments, UTR view module 260-7 identifies new poly-A site motifs and poly-A site recognition motifs based on PWM methods, sliding window methods, motif search methods, methods using motif sequence lookups, and sequence alignment methods in long sequences up to more than 10,000 bases downstream of CDS end and gene end.

In some embodiments, UTR view module 260-7 identifies the promoter motifs by combining each of the shorter promoter elements such as TATA, CAAT, and GC, and, in addition, the transcription start site (TSS) calculates promoter motif score by combining the scores of individual promoter elements such as TATA, CAAT, and GC, defining the strength of the promoter, and defines other transcriptional regulating elements such as enhancers and silencers and determining their combined scores. In some embodiments, UTR view module 260-7 determines the poly-A motifs by combining additional signals such as T/GT-rich downstream sequence elements, T-rich upstream sequence elements, G-rich auxiliary downstream elements, and TGTA elements, calculates poly-A motif score by combining the different scores of each of the elements such as T/GT-rich downstream sequence elements, T-rich upstream sequence elements, G-rich auxiliary downstream elements, and TGTA elements, and identifies and analyzes the mutations in these promoter and poly-A motifs and their implications in disease causation. In some embodiments, UTR view module 260-7 identifies subject mutations in potential gene control elements in long sequences up to more than 10,000 bases upstream of gene start and downstream of gene end, using tools provided within Genome Explorer and Splice Atlas.

BPS view module 260-8 predicts, illustrates, and analyzes BPSs in one, or multiple genes from a genome, identifies the mutations from subjects or known mutations within BPSs, and identifies the mutations from subjects or known mutations within branch point sites and their correlation with cancers and other diseases. In some embodiments, BPS view module 260-8 uses algorithm 250 (e.g., the Shapiro & Senapathy algorithm and other relevant algorithms) in genes in a genome, and represents BPSs on the gene and genomic scale of individual subjects and cohorts of subjects. In some embodiments, BPS view module 260-8 enables the discovery of frequently mutated genes within the BPS from subjects, and correlates the molecular details of the structure/function and aberrations in these genes with the phenotypes, traits, and disease or drug responses.

In some embodiments, BPS view module 260-8 predicts the splicing alterations and aberrations and their effect in splicing the transcript resulting in a defective protein based on the mutations within the branch point regions in a gene, and displays the branch point mutations and their effects on splicing (e.g., intron retention, exon skipping, and cryptic exons inclusions) in the transcripts, mRNA and protein with graphical, sequence, and tabular illustrations of the gene, RNA and protein from subjects. In some embodiments, BPS view module 260-8 displays the frequency of branch point mutations in genes in the genome in one view, and their effects on splicing (e.g., intron retention, exon skipping, and cryptic exons inclusions) in the transcripts, mRNA and protein with graphical, sequence, and tabular illustrations of the gene, RNA and protein from subjects. In some embodiments, BPS view module 260-8 identifies cryptic branch point sequences within exons and introns and throughout the gene sequence, and in genes across the genome, with varying range of score thresholds calculated based on the Shapiro & Senapathy algorithm and other available algorithms, and depicting them graphically on the gene structure, sequence, and tabular illustrations. In some embodiments, BPS view module 260-8 displays the branch point mutations in one or more genes individually or on the genome-scale in one view, from the mutation databases such as dbSNP, ClinVar, and COSMIC, and their effects on splicing (e.g., intron retention, exon skipping, cryptic exons inclusions) in the transcripts, mRNA and protein with graphical, sequence, and tabular illustrations of gene, RNA, and protein from subjects.

In some embodiments, BPS view module 260-8 displays the mutations in the cryptic branch point sites on one or more genes individually or on the genome-scale in one view, from the variant databases such as dbSNP, ClinVar, and COSMIC, and their effects on splicing (e.g., intron retention, exon skipping, cryptic exons inclusions) in the transcripts, mRNA and protein with graphical, sequence, and tabular illustrations of gene, RNA, and protein from a subject or cohort of subjects. In some embodiments, BPS view module 260-8 enables the discovery of frequently mutated genes within the cryptic branch point regions from subjects, and correlating the molecular details of the structure/function and aberrations in these genes with the phenotypes, traits, and disease or drug response, analyzes branch point mutations from a subject, overlaid on the genes to correlate their involvement in disease, and enables the identification, visualization, and deeper analysis of branch points and other regulatory elements and their cryptic versions, individually and in combinations, in a single application. In some embodiments, BPS view module 260-8 builds sub-PWMs for the non-canonical branch points surrounding the first downstream base from the 3′ intron end, enables the visualization and deeper analysis of branch points and other regulatory elements and their cryptic versions, individually and in combinations, in a single application, enables the analysis of BPS from single subjects or cohort of subjects, and enables the analysis of BPS and its combinations with different coding and regulatory elements from single subjects or cohort of subjects in a single application.

In some embodiments, BPS view module 260-8 predicts, illustrates, and analyzes a BPS in one, multiple, or genes from the genomes of organisms including the human, other animals, plants, and eukaryotic microbial organisms. In some embodiments, BPS view module 260-8 enables the user-guide of the platform such as the “About” that automatically provides context sensitive explanations for various features and applications, and “How To” that automatically provides context sensitive information of how to use particular features throughout the different sections of the platform. In some embodiments, BPS view module 260-8 provides statistics analysis for the genes in various organisms and displaying various statistics and information across the genome, enables the use of tightly coupled navigation by interlinking different sections to provide analysis of a gene, protein, or other elements and features throughout the platform, and enables an expanded version for each of the features of BPS view module 260-8, which allows users to visualize and analyze further details in graphical, tabular, and sequence illustrations.

Regulatory module 260-9 identifies promoter enhancer and silencer regions at a distance from the promoter, at the 5′ or 3′ sides of the gene or within exons and introns, or at remote locations on the same or other chromosomes. Enhancers and silencers of polyadenylation signals are also found at the 5′ or 3′ sides of the gene or within exons and introns. Enhancers and silencers of splicing are found within exons and introns and other regions of the gene. Regulators of trans-splicing may occur remotely on the same or other chromosomes. In some embodiments, regulatory module 260-9 identifies short sequence motifs that contain binding sites for transcription factors and other binding proteins and activate their target genes by binding to specific sequences. In some embodiments, regulatory module 260-9 identifies silencers that suppress the gene expression, splicing, or other processes. Although the enhancer DNA may be far from the gene in a linear way, it may be spatially close to the promoter and gene. This allows the enhancer sequence to interact with the general transcription factors and RNA polymerase II. The same mechanism holds true for silencers. Silencers are antagonists of enhancers that, when bound to its proper transcription factors called repressors, repress the transcription of the gene. In some embodiments, regulatory module 260-9 identifies an enhancer located within several hundred thousand bases upstream or downstream of the gene it regulates. Enhancers act by binding to activator proteins and not on the promoter regions. These activator proteins interact with the mediator complex, which recruits polymerase II and the general transcription factors which then begin transcribing the genes. Enhancers can also be found within introns. In addition, enhancers can be found at the exonic region of an unrelated gene, and may act on genes on another chromosome. In some embodiments, regulatory module 260-9 identifies the trans-acting splicing activator and splicing repressor proteins as well as cis-acting elements within the pre-mRNA itself such as enhancers and silencers. These sequences are located within both exons and introns that either enhance or suppress splicing. In some embodiments, regulatory module 260-9 identifies exonic splicing enhancers (ESEs) and intronic splicing enhancers (ISEs) that activate or enhance the splicing process, from within exons while intronic splicing enhancers (ISEs) and silencers (ISSs) suppress the splicing process from within introns.

In some embodiments, regulatory module 260-9 identifies cis-regulatory elements i.e., exonic and intronic splicing enhancers (ESE and ISE, respectively) and exonic and intronic splicing silencers (ESS and ISS, respectively) by recognizing specific splicing repressors and activators (trans-acting elements) that help to properly carry out the splicing process. In some embodiments, regulatory module 260-9 identifies splicing enhancers to which splicing activator proteins bind, increasing the probability that a nearby site will be used as a splice junction. These also may occur in the intron (intronic splicing enhancers, ISE) or exon (exonic splicing enhancers, ESE). In some embodiments, regulatory module 260-9 identifies an exonic splicing enhancer (ESE) consisting of ˜6 bases within an exon that enhances accurate splicing of pre-mRNA into the mRNA. Most of the activator proteins that bind to ISEs and ESEs are members of the SR protein family. Such proteins contain RNA recognition motifs and arginine and serine-rich (RS) domains. In some embodiments, regulatory module 260-9 identifies splicing silencers to which splicing repressor proteins bind, reducing the probability that a nearby site will be used as a splice junction. These can be located in the intron itself (intronic splicing silencers, ISS) or in a neighboring exon (ESS). An ESS is a short region (usually 4-18 nucleotides) of an exon, which inhibits or silences splicing of the pre-mRNA and contributes to constitutive and alternative splicing. The majority of splicing repressors are heterogeneous nuclear ribonucleoproteins (hnRNPs) such as hnRNPA1 and polypyrimidine tract binding protein (PTB).

In some embodiments, regulatory module 260-9 identifies point mutations in exons that inactivates an ESE, can create an ESS, which in turn can lead to alternative events like exon skipping and eventually a truncated protein resulting in genetic disorders. Mutations in these regions are of very high significance as these are implicated in numerous cancers and non-cancer disorders. Also, the adaptive significance of splicing silencers and enhancers is further attested by multiple studies showing that there is a strong selection in human genes against mutations that produce new silencers or disrupt existing enhancers. In some embodiments, regulatory module 260-9 identifies cryptic enhancers, and silencers also have great impact in gene expression, splicing, and translation. These cryptic regulators may be present anywhere in the genome and affect the gene expression and splicing on a large scale on account of mutational aberrations within them. Mutations in the cryptic sites may increase their scores (calculated using modified Shapiro & Senapathy algorithms and other algorithms), which may lead to suppression of gene expression or regulation of unwanted genes.

In some embodiments, regulatory module 260-9 creates a map to enable the prediction, illustration, and analysis of enhancers and silencers and their cryptic versions, and their mutational aberrations, employing the modified Shapiro & Senapathy algorithm and other relevant algorithms in any genomes including human and other organisms. In addition, it provides a platform for predicting and analyzing the effects of known mutations in these regulatory elements as well as mutations from individual subjects' genomes and the genomes from subject cohorts. In some embodiments, regulatory module 260-9 identifies regulators of trans-acting elements for gene regulation and splicing that may occur remotely on the same or other chromosomes. Splice Atlas identifies the cis-acting enhancer and silencer motifs and elements, their cryptic versions, and mutations, based on several methodologies throughout the gene, and trans-acting enhancer and silencer motifs and elements using similar methods at remote locations on the same or different chromosomes. In addition, Splice Atlas also identifies the cryptic versions of regulatory and splicing elements and mutations within them.

In some embodiments, regulatory module 260-9 identifies and illustrates mutations from a third party database such as dbSNP, ClinVar, and COSMIC and are also retrieved and overlaid over these enhancer and silencer sites. In addition, mutations from the individual subjects' genome and from a cohort of subjects are also identified and plotted over the gene plot. Enhancers and silencers for polyadenylation sites are also determined using similar methods. The details of elements including the gene regulating enhancers and silencers, splicing enhancer and silencers and their cryptic forms are illustrated on gene plots in compact and expanded view, tabular forms, and detailed sequence views. Mutations in these elements and the molecular details of aberrations are also illustrated and enabled for interpretation and analysis. In some embodiments, regulatory module 260-9 provides a map of enhancers and silencers for predicting, illustrating, and analyzing the regulatory elements in genes, identifies and analyzes the mutations from subjects in these elements, and correlating with clinical impacts. In some embodiments, regulatory module 260-9 identifies the exon and intron splicing enhancers (ESEs & ISEs) or silencers (ESSs & ISSs) by adapting the modified Shapiro & Senapathy algorithm and other relevant algorithms in genes in a genome, and representing them on the gene and genomic scale of individual subjects and cohorts of subjects. In some embodiments, regulatory module 260-9 identifies known mutations from sources such as dbSNP, ClinVar, and COSMIC, in the splicing enhancers (ESEs & ISEs) or silencers (ESSs & ISSs) in the genes of subjects and in cohorts of subjects, and their analysis in correlation with various diseases.

In some embodiments, regulatory module 260-9 identifies frequently mutated genes within the splicing enhancers or silencer regions from an individual or cohort of subjects, and correlating the molecular details of the structure/function and aberrations in these genes with the phenotypes, traits, or drug responses, and identifies mutations in the splicing enhancers or silencers responsible for aberrations involved in adverse drug reactions and affecting the efficacy of varied drugs in a subject. In some embodiments, regulatory module 260-9 displays the mutations in the splicing enhancer or silencer sites on one or more genes individually or on the genome-scale in one view, from the variant databases such as dbSNP, ClinVar, and COSMIC, and their effects on splicing (e.g., intron retention, exon skipping, cryptic exons inclusions) in the transcripts, mRNA and protein with graphical, sequence, and tabular illustrations of gene, RNA, and protein from a subject or cohort of subjects. In some embodiments, regulatory module 260-9 displays mutations in the cryptic splicing enhancer and silencer sequences on one or more genes individually or on the genome-scale in one view, from the variant databases such as dbSNP, ClinVar, and COSMIC, and their effects on splicing (e.g., intron retention, exon skipping, cryptic exons inclusions) in the transcripts, mRNA and protein with graphical, sequence, and tabular illustrations of gene, RNA, and protein from subjects. In some embodiments, regulatory module 260-9 provides interactive visualizations and analytical capabilities for focusing on the mutations in various splicing enhancers or silencer regions individually on gene structures and on a genomic scale, and facilitating the ability to perform analysis on enhancers and silencers across subjects.

In some embodiments, regulatory module 260-9 identifies cryptic splicing enhancer and silencer sequences within exons and introns and throughout the genes across the genome, with varying range of score thresholds calculated based on the modified Shapiro & Senapathy algorithm and other available algorithms, and depicting them graphically on the gene structure, sequence, and tabular illustrations. In some embodiments, regulatory module 260-9 identifies the mutations from subjects on the cryptic splicing enhancer and silencer sequences, and their effects on splicing (e.g., intron retention, exon skipping, and cryptic exons inclusions) in the transcripts, mRNA and protein structures and functions. In some embodiments, regulatory module 260-9 predicts and identifies regulators of trans-splicing that may occur remotely on the same or other chromosomes, identifies cis-acting enhancer and silencer motifs and elements based on PWM methods, uses sliding window methods, motif search methods, methods using motif sequence lookups, and sequence alignment methods throughout the gene, and identifies trans-acting enhancer and silencer motifs and elements based on PWM methods, sliding window methods, motif search methods, methods using motif sequence lookups, and sequence alignment methods throughout the gene on remote locations on the same or different chromosomes.

In some embodiments, regulatory module 260-9 analyzes enhancers and silencers in gene expression, splicing and translation, and their cryptic versions, and its combinations with different coding and regulatory elements from single subjects or cohort of subjects in a single application. In some embodiments, regulatory module 260-9 includes a platform for predicting, illustrating, and analyzing the enhancer and silencer sequences in one, multiple, or genes from the genomes of organisms including the human, other animals, plants, and eukaryotic microbial organisms. In some embodiments, regulatory module 260-9 enables a user-guide of the platform such as the “About” that automatically provides context sensitive explanations for various features and applications, and “How To” that automatically provides context sensitive information of how to use particular features throughout the different sections of the platform. In some embodiments, regulatory module 260-9 provides statistics analysis for all the genes in various organisms and displays various statistics and information across the genome, enables the use of tightly coupled navigation by interlinking different sections to provide analysis of a gene, protein, or other elements and features throughout the platform, and enables an expanded version for each of the features of enhancer/silencer view, which allows users to visualize and analyze further details in graphical, tabular, and sequence illustrations.

In some embodiments, regulatory module 260-9 includes a modified version of the Shapiro & Senapathy algorithm. In some embodiments, regulatory module 260-9 includes modified versions of other splicing algorithms such as MaxEntScan, NNSplice, and Human Splicing Finder. In some embodiments, regulatory module 260-9 detects each of the different regulatory elements (promoter boxes such as TATA box, CAT box, GC box), promoter element, transcription initiator, branch point, exon splice enhancer and silencer (ESE & ESS), intron splice enhancer and silencer (ISE & ISS, poly-A site) based on the specific position weight matrix (PWM) derived from the respective consensus sequence frequencies and sequence length of each regulatory element. In some embodiments, regulatory module 260-9 detects the cryptic versions of each of the different regulatory elements (promoter boxes -TATA box, CAT box, GC box-, promoter element, transcription initiator, branch point, exon splice enhancer and silencer -ESE & ESS-, intron splice enhancer and silencer -ISE & ISS-, and poly-A site). In some embodiments, regulatory module 260-9 detects the different regulatory elements, and their cryptic versions, throughout the gene, and throughout multiple genes or all genes within a genome.

In some embodiments, regulatory module 260-9 detects the different regulatory elements, and their cryptic versions, throughout the exons within a gene, throughout multiple genes or all genes within a genome, detects the different regulatory elements, and their cryptic versions, throughout the introns within a gene, throughout multiple genes or all genes within a genome, and detects the different regulatory elements, and their cryptic versions, throughout the un-transcribed (promoter and upstream, poly-A site and downstream) and un-translated regions (5′ and 3′ UTR) within a gene, throughout multiple genes or all genes within a genome. In some embodiments, regulatory module 260-9 detects the different regulatory elements, and their cryptic versions, throughout the intergenic regions of a genome, detects cryptic exons, throughout the exons within a gene, throughout multiple genes or all genes within a genome, detects cryptic exons throughout the un-transcribed (promoter and upstream, poly-A site and downstream) and un-translated regions (5′ and 3′ UTR) within a gene, throughout multiple genes or all genes within a genome. In some embodiments, regulatory module 260-9 detects cryptic exons throughout the introns within a gene, throughout multiple genes or all genes within a genome, detects cryptic exons throughout the intergenic regions of a genome, and identifies deleterious mutations within splice sites to detect the deleterious mutations within each of the different regulatory elements (promoter boxes -TATA box, CAT box, GC box-, promoter element, transcription initiator, branch point, exon splice enhancer and silencer -ESE & ESS-, intron splice enhancer and silencer -ISE & ISS-, poly-A site) based on the specific position weight matrix (PWM) derived from the respective consensus sequence frequencies and sequence length of each regulatory element.

In some embodiments, regulatory module 260-9 detects deleterious mutations within each of the different regulatory elements (promoter boxes: TATA box, CAT box, GC box), promoter element, transcription initiator, branch point, exon splice enhancer and silencer (ESE & ESS), intron splice enhancer and silencer (ISE & ISS), and poly-A site, and detects deleterious mutations within the cryptic versions of each of the different regulatory elements (promoter boxes (TATA box, CAT box, GC box), promoter element, transcription initiator, branch point, exon splice enhancer and silencer (ESE & ESS), intron splice enhancer and silencer (ISE & ISS), and poly-A site). In some embodiments, regulatory module 260-9 detects deleterious mutations within the different regulatory elements, and their cryptic versions, throughout the gene, and throughout multiple genes or all genes within a genome, detects deleterious mutations within the different regulatory elements, and their cryptic versions, throughout the exons within a gene, throughout multiple genes or all genes within a genome, and detects deleterious mutations within the different regulatory elements, and their cryptic versions, throughout the introns within a gene, throughout multiple genes or all genes within a genome.

In some embodiments, regulatory module 260-9 detects deleterious mutations within the different regulatory elements, and their cryptic versions, throughout the un-transcribed (promoter and upstream, poly-A site and downstream) and un-translated regions (5′ and 3′ UTR) within a gene, throughout multiple genes or all genes within a genome, detects deleterious mutations within the different regulatory elements, and their cryptic versions, throughout the intergenic regions of a genome, detects deleterious mutations within cryptic exons throughout the exons within a gene, throughout multiple genes or all genes within a genome, and detects deleterious mutations within cryptic exons throughout the introns within a gene, throughout multiple genes or all genes within a genome. In some embodiments, regulatory module 260-9 detects: deleterious mutations within cryptic exons throughout the un-transcribed (promoter and upstream, poly-A site and downstream) and un-translated regions (5′ and 3′ UTR) within a gene, throughout multiple genes or all genes within a genome, detects deleterious mutations within cryptic exons throughout the intergenic regions of a genome, finds mutations within the new genes discovered within the introns and intergenic regions, and identifies splice sites (such as MaxEntScan, NNSplice, Human Splicing Finder) to detect each of the different regulatory elements (promoter boxes (TATA box, CAT box, GC box), promoter element, transcription initiator, branch point, exon splice enhancer and silencer (ESE & ESS), intron splice enhancer and silencer (ISE & ISS), poly-A site, based on the specific position weight matrix (PWM) derived from the respective consensus sequence frequencies and sequence length of each regulatory element.

In some embodiments, ncRNA map module 260-10 identifies and illustrates ncRNA genes from the human genome, and their splicing and processing into the mature functional RNA molecules in tabular, graphical, and sequence illustrations, and creates a repository for the non-coding RNA genes platform containing all possible information for ncRNA genes in a genome such as exon details with the genomic position of the exons, transcript details, exon length, splicing and maturation processes, and consequences of the mutations. In some embodiments, ncRNA map module 260-10 identifies mutations in the non-coding RNA genes by modifying and applying the Shapiro & Senapathy algorithm and other relevant algorithms across the gene and genomic scale from individual subjects and in a cohort of subjects, and enabling the clinicians to correlate the mutations in non-coding RNA genes that drive disease pathogenesis, and identifies mutations in the regulatory elements of the non-coding RNA genes responsible for disease-causing, adverse drug reactions and affecting the efficacy of various drugs in a subject. In some embodiments, ncRNA map module 260-10 identifies known disease-causing mutations in different ncRNA genes, and using them to predict or diagnose mutations and diseases from the subject genome, parses the identified mutations in non-coding RNA genes against the curated Genome Explorer proprietary mutation database, enabling to distinguish and categorize the known and novel mutations of non-coding RNA genes reported in the individual and cohort subjects, and identifies structural and functional motifs and elements in the non-coding (nc) RNA genes (rRNA, tRNA, miRNA, snRNA, snoRNA, siRNA, lncRNA).

In some embodiments, ncRNA map module 260-10 identifies disease-causing mutations in different ncRNA genes, predicting or diagnosing, mutations and diseases from the subject genome, and known disease-causing mutations in different ncRNA genes, using them to predict or diagnose mutations and diseases from the subject genome. In some embodiments, ncRNA map module 260-10 identifies sequence signals for processing different ncRNA genes to their mature forms using the modified Shapiro & Senapathy and other algorithms based on consensus, PWMs, and other relevant parameters for all ncRNA genes, and compares subject ncRNA gene sequences with reference sequences to identify mutations using modified Shapiro & Senapathy and other relevant algorithms based on the score difference between the normal and the mutated signals. In some embodiments, ncRNA map module 260-10 identifies subjects with frequently occurring mutations in the structural and functional motifs and elements in the non-coding (nc) RNA genes (rRNA, tRNA, miRNA, snRNA, snoRNA, siRNA, lncRNA), enables the visualization and analysis of variability within the ncRNA sequence positions to determine disease associations, and defines the allowed (green) and non-allowed (red) regions of the positive-negative signature of an ncRNA gene from the alignment of various types of ncRNA genes, and determines the pathogenicity or deleteriousness of a variant by its occurrence in the positive (green) or negative (red) region of the ncRNA signature.

In some embodiments, ncRNA map module 260-10 displays non-redundant bases from the multiple sequence alignment of ncRNA genes from various organisms in one color (e.g., green), and all other bases in another color (e.g., red), showing a map of allowed (positive) and non-allowed (negative) nucleotide substitution space across the sequence, indicating variants that may result in a viable or defective regulatory RNA. In some embodiments, ncRNA map module 260-10 determines that the deleterious mutations would fall within the negative region (red) and that the benign or likely pathogenic mutations would fall within the positive region (green), and applying this finding in testing and determining if a given variant is deleterious or not, and determines whether the impact and clinical significance of the mutations is deleterious or not, based on the occurrence of the altered base within the negative space or the positive space, thereby showing where the actual mutations occur by color codes. In some embodiments, ncRNA map module 260-10 provides interactive visualizations and analytical capabilities for focusing on the mutations in various non-coding RNA genes individually on gene structures and on a genomic scale, facilitating the ability to perform non-coding RNA gene analysis across individual and multiple subjects and cohorts, which may be involved in the regulation of gene expression, splicing, transcriptional and translational control, chromatin remodeling, and cell proliferation. In some embodiments, ncRNA map module 260-10 identifies new disease-causing mutations in non-coding RNA genes based on individual and cohort analysis, and providing a range of therapeutic targets and enabling and exploiting the development of RNA-based therapeutics, enables the search option for genes from various gene panels such as disease panels, and other user given gene panels, and enables toggle options for displaying the graphical illustrations of details of every ncRNA gene and plotting the mutations in an expanded view.

In some embodiments, ncRNA map module 260-10 identifies and analyzes exon splicing of an ncRNA gene, plotting the subject's mutation or any known mutations in the ncRNA genes from different databases such as dbSNP, ClinVar, and COSMIC, the effect of mutations such as suppression of gene expression, splicing, and transcriptional regulation based on the indigenous algorithm of ncRNA MAP, and analyzes subject mutations overlaid on the ncRNA genes to correlate their involvement in disease. In some embodiments, ncRNA map module 260-10 identifies mutations from databases such as ClinVar, dbSNP, and COSMIC on the ncRNA genes to correlate their involvement in disease, enables the analysis of ncRNA from single subjects or cohort of subjects, enables the analysis of ncRNA mutations in various combinations of different regulatory elements from single subjects or cohort of subjects in a single application, and identifies the non-coding RNA sequences in one, multiple, or genes from the genomes of organisms including the human, other animals, plants, and eukaryotic microbial organisms. In some embodiments, ncRNA map module 260-10 enables the user-guide of the platform such as the “About” that automatically provides context sensitive explanations for various features and applications, and “How To” that automatically provides context sensitive information of how to use particular features throughout the different sections of the platform, provides ncRNA map statistics genes in various organisms and displays various statistics and information across the genome. In some embodiments, ncRNA map module 260-10 enables tightly coupled navigation by interlinking different sections to provide analysis of a gene, protein, or other elements and features throughout the platform, and enables an expanded version for each of the features of the ncRNA map, which allows users to visualize and analyze further details in graphical, tabular, and sequence illustrations.

Dark matter module 260-11 identifies protein genes within the dark matter genome using various algorithms such as Shapiro & Senapathy, Splice Atlas Splice Code, GenScan, Augustus, and GeneID, and identifies ncRNA genes within the dark matter genome using various algorithms. In some embodiments, dark matter module 260-11 identifies potential domains from the protein-coding genes of the dark matter genome using PfamScan and other algorithms, and applies each of modules 260, on newly found genes to integrate relevant data and information into database 252.

Application 222 may be installed by server 130 and perform scripts and other routines provided by server 130 to display graphic payload 225 provided by genome sequence analysis engine 242. In some embodiments, graphic payload 225 may include a mark for a mutation of the nucleotide string on a positive signature and a negative signature of a protein domain. In some embodiments, graphic payload 225 may include i-icons, mouse hovers with context sensitive pop ups for the user, pull down menus, sliding windows and scales, active tabs and buttons, and other interactive elements that enable the user to retrieve more detailed information. In some embodiments, graphic payload 225 may enable toggle options for displaying the graphical illustrations of a selected gene or portion of the human genome, and plotting the corresponding mutations in an expanded view. Further, graphic payload 225 may enable a user-guide tab (e.g., including an “About” option that automatically provides context sensitive explanations for various features and applications, and a “How To” that automatically provides context sensitive information of how to use and navigate particular features throughout the different sections of modules 260). Embodiments as disclosed herein enable the use of tightly coupled navigation features interlinking different sections and modules 260 to provide analysis of a selected gene, protein, or other elements and features throughout the platform.

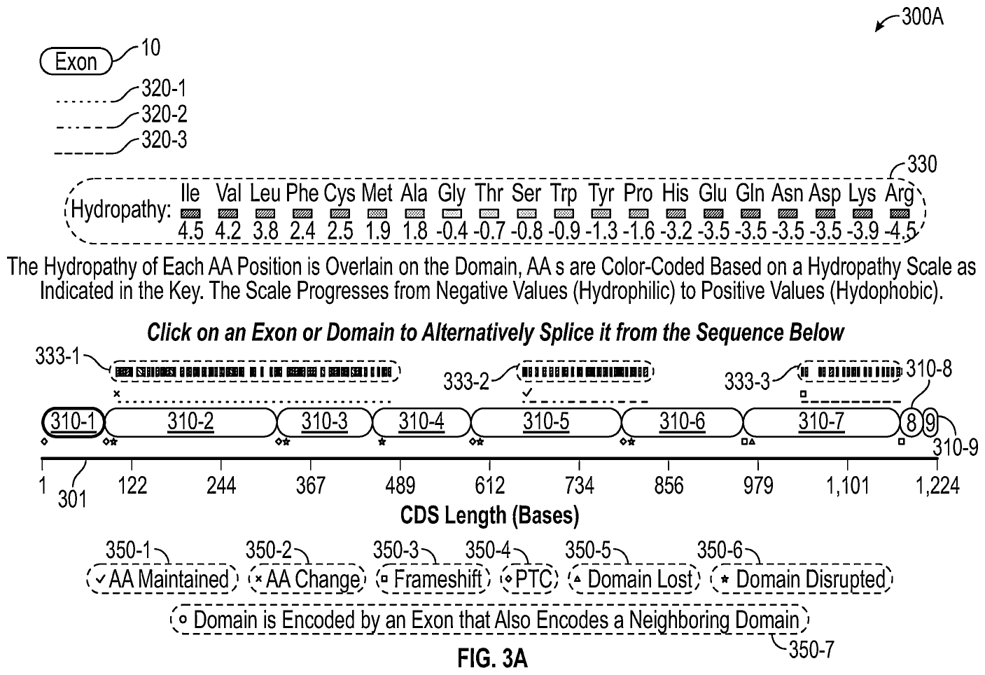

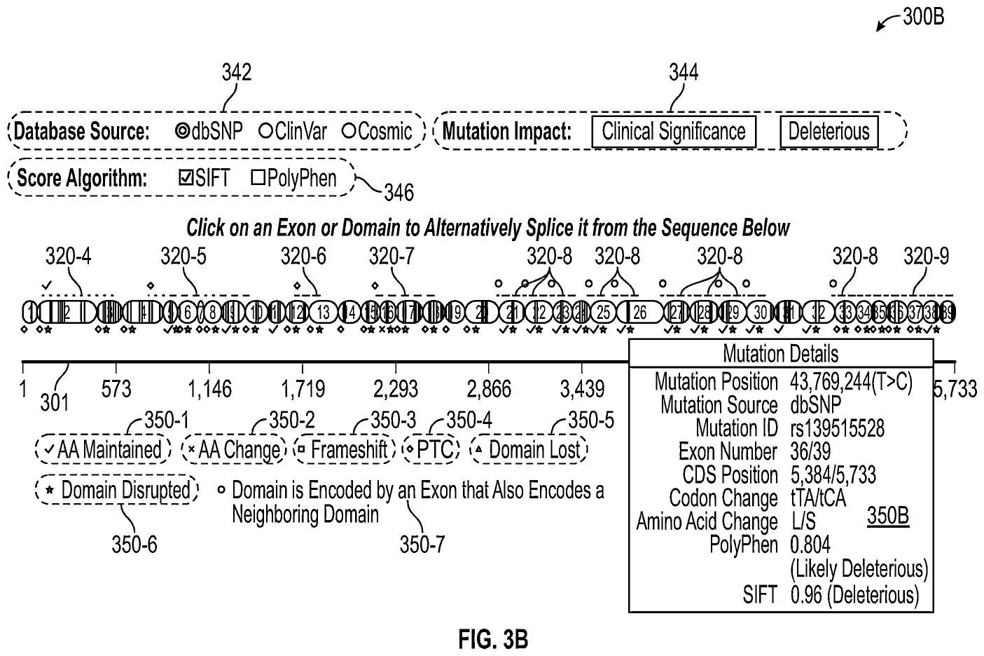

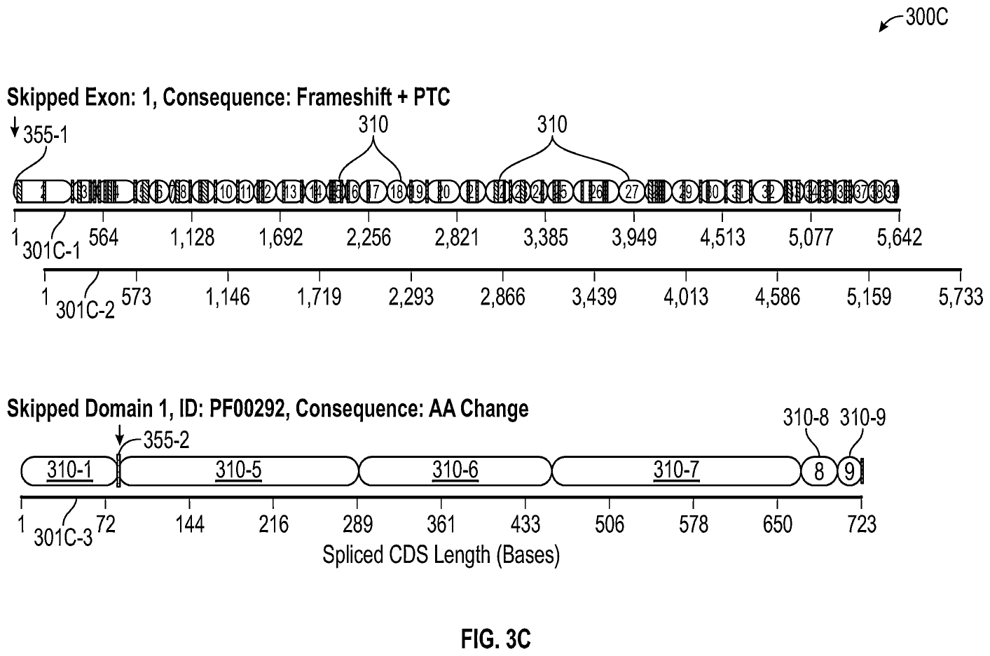

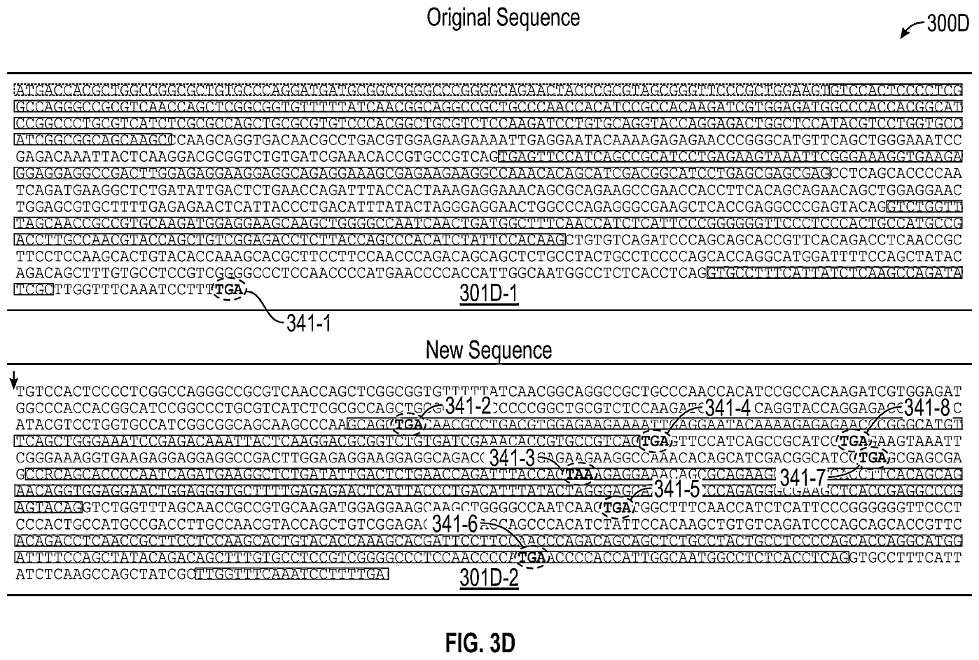

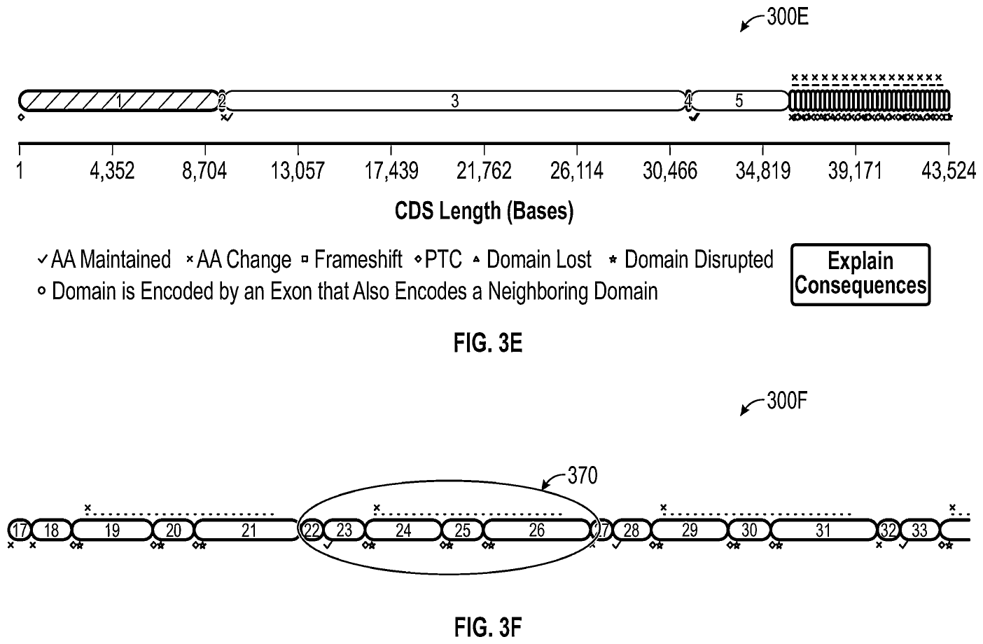

FIGS. 3A-3F illustrate details of exon splices 300A, 300B, 300C, 300D, 300E, and 300F (hereinafter, collectively referred to as “exon splices 300”), according to embodiments disclosed herein. In some embodiments, exon splices 300 may be provided by an exon splice module interacting with a genome sequence analysis engine, as disclosed herein (e.g., exon splice module 260-1, and genome sequence analysis engine 242). Exon splices 300 may include an “after splicing view” to illustrate the consequence of excluding one or more exons that correspond to a protein domain during RNA splicing. In some embodiments, the “after splicing view” illustrates the disrupted protein product post-splicing, including the spliced exon structure and the protein product. The disrupted protein may undergo tolerated changes or destructive changes. Exon splices 300 indicate the protein changes on both the micro (nucleotide scale) and the macro (protein structure) scales, enabling disease and biological correlations.

In some embodiments, the coding exon-domain plot for the selected gene is visualized with exons in grey rectangles and domains overlaid on them. Domains are predicted using a search engine to search a protein sequence for the presence of domains encoded by the gene. The consequences of splicing any of the exons or set of exons coding for a particular domain are predicted based on the S&S algorithm and the codon degeneracy principle. The reading frame of the resultant ORF after splicing out every exon individually, is checked for its correctness. If the frame of the ORF is shifted due to exon excision, introduction of a premature termination codon (PTC), or the deletion of domain coding sequence (single domain or multiple domains) are combined to depict multiple possible consequences.

Exon splices 300 may be provided for genes from various panels for different diseases, with user preferences accommodated for diseases list, gene names, and transcript identifiers. The most probable and destructive splice events are indicated for the chosen disease diagnosis. Exon splices 300 may indicate protein isoforms associated with a selected sequence of exons 310 codifying different protein domains 320-1, 320-2, and 320-3 (hereinafter, collectively referred to as “protein domains 320”). Exons 310-1, 310-2, 310-3, 310-4, 310-5, 310-6, 310-7, 310-8, and 310-9 (hereinafter, collectively referred to as “exons 310”) include portions of a nucleotide string 301 coding amino acids in a protein having protein domains 320. Exon splices 300A, 300B, and 300C include after splicing views of exons 310. Exon splice 300A includes a hydropathy view of protein domains 320, and exon splice 300D includes a sequence view listing the nucleotide string of the exon chain. Exon splices 300 may be provided in a graphic payload of an application running in a client device and hosted by a genome sequence analysis engine in a server, as disclosed herein. For example, exon splices 300 may include a display of an amino acid hydropathy chart 330, listing the hydrophobicity and/or hydrophilicity of each amino acid. The hydro section aids in visualizing the signature of the selected Pfam ID based on the values of their hydropathy index. The signature plot in this section is color-coded based on the hydropathy index scale, where hydrophobic amino acids are represented in shades of red and hydrophilic amino acids are shown in shades of blue. Accordingly, a hydropathy pattern 333-1, 333-2, and 333-3 (hereinafter, collectively referred to as “hydropathy patterns 333”) may be displayed for each of the protein domains 320. Hydropathy patterns 333 depict hydropathy index values determined by various methods along the amino acids sequence of the selected transcript. In some embodiments, hydropathy patterns 333 may be indicated using a sliding window. In some embodiments, hydropathy patterns 333 may display the amino acids in color codes based on the hydropathy nature of amino acids, and the exon splice module may enable a mouse hover on each of the amino acids in the graphic payload supporting exon splice 300C so the user may view the corresponding codon in the nucleotide string.

In some embodiments, a hydropathy score in hydropathy pattern 333 may be calculated by a moving average of several adjacent amino acids, and exon splices 300 may enable mouse hover on the amino acid sequence or plot to view the hydropathy values. In some embodiments, exon splices 300 illustrate a hydropathy pattern 333 as a pattern of hills and valleys, showing the balance between hydrophobic and hydrophilic amino acids encoded by exons 310. In some embodiments, hydropathy pattern 333 includes a disruptive hydropathy indicating an imbalance in the hydropathy nature of amino acids caused by mutation.

FIG. 3A and FIG. 3B illustrate exon splices 300A and 300B including mutations and other protein effects such as amino acid maintained 350-1, amino acid change 350-2, frameshift 350-3, premature termination codon (PTC) 350-4, domain lost 350-5, domain disrupted 350-6, and domain encoded by an exon that also encodes a neighboring domain 350-7 (hereinafter, collectively referred to as “protein effects 350”) in the protein sequence. Exon splice 300B also includes a tab 342 to select a database source (cf. database 252, e.g., dbSNP ClinVar, COSMIC), and a tab 344 for identifying a mutation impact (e.g., clinical significance=deleterious) for a selected mutation. The mutations curated for the selected gene can be depicted on the plot by configuring the mutations toggle. The mutation details fetched from the respective databases are displayed on hover of a particular mutation. The clinical significance may include ontology information of a disease and a disease phenotype. In some embodiments, exon splice 300B may display a mutation details window 350B indicating details of a selected mutation such as mutation position, mutation source (e.g., database 252), mutation ID, exon number (where the mutation occurs), CDS position, codon change, amino acid change, and a score factor for the mutation calculated by a selected scoring algorithm. Exon splice 300B also provides for the user a tab 346 to select the score algorithm to evaluate the mutation score factor (e.g., SIFT, PolyPhen, cf. algorithm 250). Exon splices 300A and 300B illustrate the locations of frameshifts, premature stop codons, and amino acid changes that lead to specific sequence alterations caused by exon exclusion, and provide graphical, tabular, and sequence view with pop-up boxes, mouse hovers, and context sensitive explanations for the user.

Protein isoforms include different protein variants that arise due to the rearrangement of the intron-exon elements during transcription, splicing, and translation. These isoforms pave the way for proteins with different structure, function, and cellular properties from a given gene, and in turn, increase the diversity of human proteins. Exon splices 300 are the result of an algorithm (cf. algorithm 250, e.g., including a Shapiro & Senapathy formulation) to predict whether inherent exon skipping events arising through potentially viable or destructive alternative splicing events, maintain or destroy the open reading frame of a gene, and thus have the potential to produce a viable or defective protein.

The algorithm predicts the outcomes for multiple exon or domain coding exon skipping events in a human gene and analyzes the downstream effect of events on the reading frame of the gene and the translated protein. Mutations such as frameshifts 350-3, premature stop codons, and amino acid changes 350-2 that cause protein alterations are predicted by mapping with a reference human genome from a database (e.g., database 252, including a Pfam database) to locate protein domains 320. Hampered functionality is also predicted by diagnosing the domain-disabling mutations or exon skipping 355-1 or 355-2 that would result in a damaged protein (cf. FIGS. 3C-3D).

In some embodiments, the exon splice module indicates the consequences of a mutation in a protein and their molecular defectiveness (e.g., tab 344). For example, a defective protein may result from a defective gene due to a splice site mutation that leads to exon skipping 355-1. Thus, an exon splice module as disclosed herein may determine the consequence of splicing mutations in a subject, leading to splicing aberrations. In some embodiments, the exon splice module may interact with an alternative splice module (e.g., alternative splice module 260-4) to determine exons 310 and protein domains 320 that may include viable alternative splicing. Further, the exon splice module and alternative splice module may indicate which exons 310 and protein domains 320 would introduce unintended consequences and affect (negatively) a protein functionality.

Exon splices 300 include exons 310 in grey blocks and domains 320 overlaid on them. In some embodiments, domains 320 may be predicted using an algorithm (e.g., PfamS can), to search a protein sequence for the presence of domains 320-4, 320-5, 320-6, 320-7, 320-8, and 320-9 (hereinafter, collectively referred to as “domains 320”) encoded by a selected gene. In some embodiments, the consequences of splicing any of exons 310 coding for a particular domain 320 are predicted based on the Shapiro & Senapathy algorithm and a codon degeneracy principle. The resulting open reading frame (ORF) after splicing out every exon individually, is checked for its correctness. When the ORF is shifted due to exon excision, introduction of PTC 350-4, or the deletion of domain coding sequence 350-5 (single domain or multiple domains) are combined to depict multiple possible consequences.

FIG. 3C illustrates an after splicing view of exon splice 300C. In some embodiments, exon splice 300C may include a skipped exon 355-1 (e.g., exon 310-1 in exon splice 300A). Accordingly, nucleotide string 301C-1 starts in a position corresponding to exon 310-2. In some embodiments, skip exon 355-1 may be the result of frameshift 350-3 combined with PTC 350-4. In some embodiments, exon splice 300C includes a skipped protein domain 355-2 (e.g., protein domain 320-2). In some embodiments, a nucleotide string 301C-2 may start at a different position marked in red (e.g., a stop codon) within exon 310-2. In some embodiments, a skipped protein domain 355-2 may be the result of overlapping domains, domain disruption 350-6, and domain skipping (or lost) 350-5, associated with a nucleotide string 301C-3.

FIG. 3D illustrates a view tab of exon splice 300D including a complete coding sequence, before splicing 301D-1, and after splicing 301D-2 (hereinafter, collectively referred to as “nucleotide strings 301D”) of the selected transcript. Exon splice 300D displays a “sequence view” of nucleotide strings 301D representing stop codons 341-1, 341-2, 341-3, 341-4, 341-5, 341-6, 341-7, and 341-8. In some embodiments, the exon splice module may also display a pathogenicity score for mutations 341, marked by an in-silico classifier. In some embodiments, the user may request a tabular illustration displaying information for selected protein domains 320 and their respective exons 310, such as encoding domain name, Pfam ID (domain identifier), Start/End within exons, Start/End within transcript, and the exons coding for the domain. Table 1 below shows an exemplary table providing a summary of some of mutations 341 and their consequence (cf. protein effects 350).

TABLE I

| Original sequence | New sequence | Consequence |
|---|---|---|
| ATGTGCAATTCCTGA | ATGTATTCCTGA | AA change |
| ATGTGCAAGTCCTGA | ATGTAGTCCTGA | AA change + PTC |
| ATGTGCAATTCCTGA | ATGAATTCCTGA | AA maintained |
| ATGTGCACGTCCTGA | ATGTACGTCCTGA | Frameshift |
| ATGTGCAAGTCCTGA | ATGTAAGTCCTGA | Frameshift + PTC |
| ATGTGCACTGACTGA | ATGTACTGACTGA | Frameshift + PTC |

Exon splices 300 aid in determining if the alternate splicing of exons encoding a domain is genuine based on if such splicing leads to PTC or frameshift in the protein sequence, or does not alter the protein sequence's frame thus maintaining the downstream sequence. This approach can thus identify genuine alternative splicing events or spurious events, incorrectly annotated due to methodological difficulties. Exon splices 300 thus identify the genuine and spurious alternative splicing occurring in all of the human genes and catalogues them as biological alternative splicing. It also enables identification of defective splicing due to various splice site mutations and the defective proteins leading to diseases.

Using information collected by exon splices 300 for any selected gene or portion of the human genome, the exon splice module may create a repository containing exon details with the encoding domains, genomic position of the exons, transcript details, exon length, protein structure of the protein domain, amino acid sequence, hydropathy index for each of the amino acids, and consequences of the exon splicing and mutations. Accordingly, embodiments as disclosed herein enable the search option for genes from various gene panels such as disease panels, drug metabolizing gene (DMG) panel, the American College of Medical Genetics and Genomics (ACMG) gene panel, and other user given gene panels.

FIGS. 3E and 3F illustrate a repeating pattern of exons and a domain in the gene MUC16 in an exon splice map, according to some embodiments.

FIG. 3F illustrates a pattern 370 with a consecutive repetition of five exons along with the domain encoded by three of the five exons. Embodiments as disclosed herein enable the identification of pattern 370, which may have clinical relevance across multiple maps.

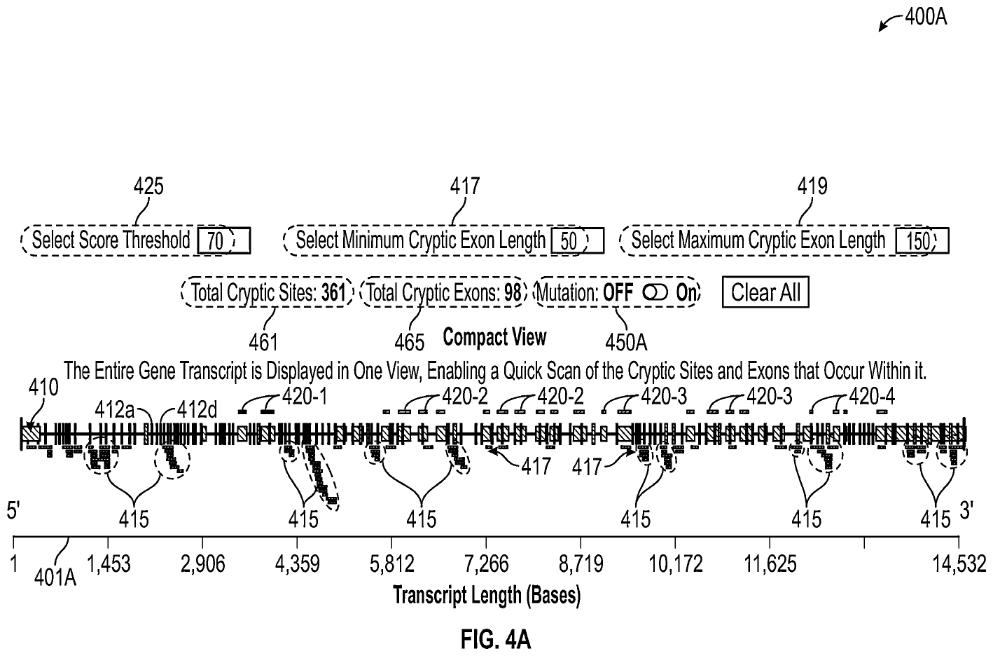

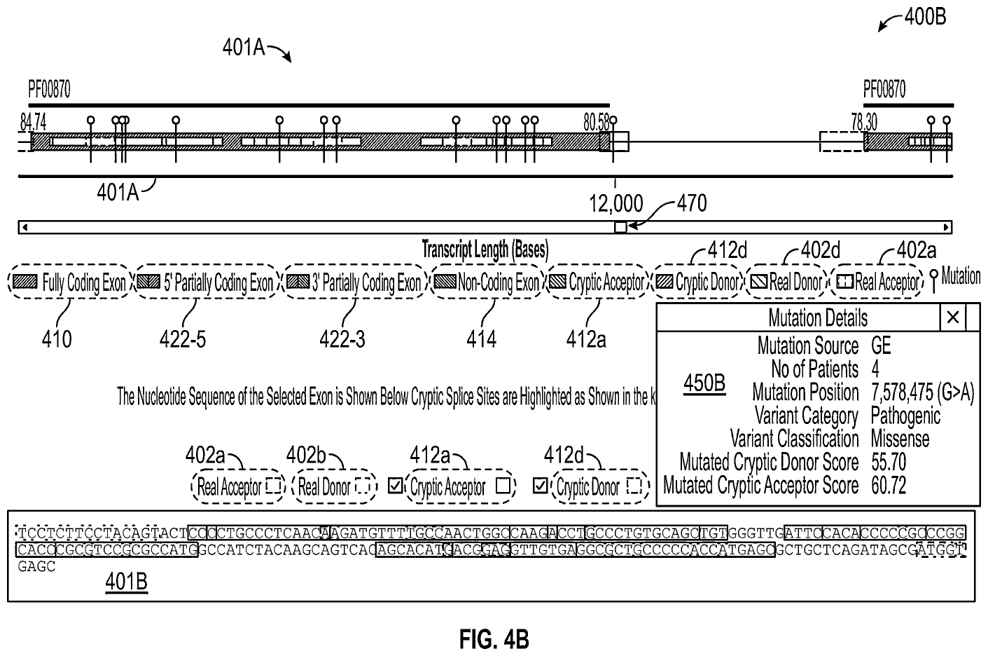

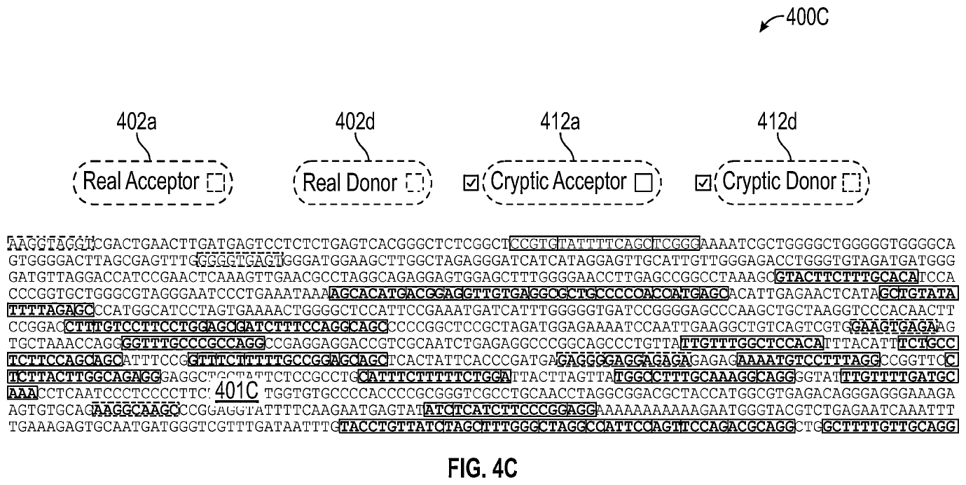

FIGS. 4A-4C illustrate details of cryptic splices 400A, 400B, and 400C (hereinafter, collectively referred to as “cryptic splices 400”), according to embodiments disclosed herein. Cryptic splices 400 may be provided by a cryptic splice module, as disclosed herein (cf. cryptic splice module 260-2). A cryptic splice site (CSS) is defined as a sequence of 15 bases for acceptors 412a and 9 bases for donors 412d (hereinafter, collectively referred to as CSS 412) that match closely with the real splice sites in sequence regions other than the real sites, anywhere within a nucleotide string 401A. A cryptic exon 415 is defined as a sequence between cryptic acceptor 412a and a cryptic donor 412d with at least one of the open reading frames (ORF) 417 between them. In some embodiments, CSS 412 is also formulated by modifying other relevant splice site prediction algorithms to predict CSS 412 for the different regulatory elements by using position weight matrix (PWM) methods, consensus sequences, and sequence lengths specific for the different elements respectively. Different protein domains 420-1, 420-2, 420-3, and 420-4 (hereinafter, collectively referred to as “protein domains 420”) are also illustrated.

The user selects a gene based on the search criteria from the drop-down list enabled under each of the search options such as genes, clinical associations, number of cryptic sites and cryptic exons. The cryptic sites and cryptic exons are depicted on the gene plot along with their scores, sequence and other additional information and presented as the gene view, sequence view, and table view. CrypticSplice enables the user to modify the cryptic site score threshold and cryptic exon length criteria to analyze the CrypticSplice map of the selected gene. Real exons and splice sites are displayed in the selected transcript as shown in the key. Any cryptic splice sites and cryptic exons that occur within the transcript are also displayed. A cryptic splice site is defined as a sequence of 15 bases for acceptors and 9 bases for donors, which has a Shapiro-Senapathy algorithm score that is above the selected score threshold. A cryptic exon is defined as a sequence between a cryptic acceptor and cryptic donor that falls within the selected length range.

FIG. 4A illustrates cryptic splice 400A, according to some embodiments.

FIG. 4B illustrates cryptic splice 400B including CSSs 412 that pass a selected score threshold and are detected and mapped onto the gene sequence. The scores of real splice sites (e.g., real acceptor 402a and real donor 402d, collectively referred hereinafter as “real splice sites 402”), real exons 410, cryptic splice sites 412, and cryptic exons 415 are shown on a nucleotide string 401A, 401B, or 401C (hereinafter, collectively referred to as “nucleotide strings 401”), creating a landscape of real and cryptic splice scores across the gene. These scores can be used to predict where erroneous splicing may occur if a mutation weakens a real splice site or strengthens a cryptic splice site. They also indicate the occurrence of alternative splicing positions in the gene during biologically mediated alternative splicing, and alternative splicing aberrations due to mutations. The cryptic splice module, using cryptic splices 400, reliably identifies hidden splice sites and exons in a selected gene, exposing likely locations that the splicing machinery will target under different biological and disease conditions.

Cryptic splice 400B enables a user driven approach to identify and correlate the mutations in cryptic acceptor 412a and cryptic donor 412d, and cryptic exons from any subject exhibiting any cancer or non-cancer disorders. A fully coding exon 410 is delimited on its 5′ end by a 5′ partially coding exon 422-5 and on its 3′ end by a 3′ partially coding exon 422-3. In some embodiments, a non-coding exon 414 may also be indicated. A slide button 470 may enable the user to pan nucleotide string 401A in either direction (towards the 5′ and 3′ ends) for convenience. In some embodiments, cryptic splice 400B enables a pattern analysis of variations in cryptic splice sites and cryptic exons for different transcripts of a given gene, and across different genes. In some embodiments, cryptic splice 400B displays mutations 450B from one or more subjects within the real and cryptic splice sites and exons, and determining the pathogenicity of these mutations by comparing the scores obtained in the normal sequence, and displaying the mutations within any of these features and genetic elements in color codes, graphical, tabular, and sequence illustrations. In some embodiments, cryptic splice 400B identifies the consequences of mutation in these structures of the transcripts and the genes with graphical and sequence illustrations, plotting subject mutations in a real or cryptic splice and exonic regions, and the known mutations from different databases such as dbSNP, ClinVar, and COSMIC, and categorized into clinical significance, molecular consequence, variation type, and pathogenicity based on the SIFT and/or PolyPhen scores. In some embodiments, cryptic splice 400B overlays the subject(s) mutations on the gene and the genome, comparing them with the known mutations from the databases, by visual illustrations and analytical tools in graphical, tabular, and sequence views with pop-up boxes, mouse hovers, and context sensitive explanations. In some embodiments, cryptic splice 400B displays the exon-intron structure of a selected gene including the promoters, UTRs, and poly-A sites, and overlaying the cryptic donor and acceptor sites and cryptic exons, as well as the subjects' variants and mutations across these features on the entire gene in a graphical display of the gene.

FIG. 4C illustrates cryptic splice 400C, according to some embodiments.

In some embodiments, individual exons and introns, un-transcribed and un-translated regions of the selected transcript of a given gene are analyzed independently. These sequences are split into acceptors (15 bases) and donors (9 bases) by several methods including sequence PWM methods using the Shapiro & Senapathy algorithm, and scores are calculated for each 15/9 mer (e.g., each acceptor/donor pair). The sites having 15/9 mer score higher than the cut-off threshold score of 50 are considered as cryptic sites. The cryptic splice module may use other methods such as sliding window methods, motif search methods, methods using motif sequence lookups, and sequence alignment methods to detect and analyze CSSs 412. Furthermore, Splice Atlas has discovered that different sequence lengths for donor and acceptor splice sites may be more optimal when compared with 15/9 bases, which are also used.

For each transcript, valid CSSs 412 are taken as study sequences. Cryptic exons 415 are formed from the last base of a cryptic acceptor 412a to the first three bases of a cryptic donor 412d. In some embodiments, a cryptic splice module as disclosed herein combines each cryptic acceptor site 412a with each of the cryptic donor sites 412d that occur within a chosen exon length limit. The scores are calculated for each exon possibility using a suitable algorithm (e.g., algorithm 250, including a Shapiro & Senapathy algorithm). In some embodiments, sequences having lengths between a minimum cryptic exon length 417 and a maximum cryptic exon length 419 (e.g., 50 and 500 bases) and a score higher than a minimum cut-off threshold score 425 (e.g., 50) are considered as cryptic exons 415. CSSs 412 also enable methodological variations of forming cryptic exons 415. In some embodiments, cryptic splices 400 may predict cryptic exons 415 based on the cryptic donor and acceptor splice site scores using equal or unequal weights for the donor and acceptor scores, and assigning a score for cryptic exons 415.

Cryptic splices 400 may identify and map a CSS 412 and a cryptic exon 415 in the human genome according to score threshold 425 within nucleotide strings 401A and 401B (hereinafter, collectively referred to as “nucleotide strings 401”). The scores of real splice sites 402, real exons 410, cryptic splice sites 412, and cryptic exons 415 are also shown, creating a landscape of real and cryptic splice scores across the gene. These scores can be used to predict where the spliceosome may accidentally turn if a mutation weakens a real splice site 402 or strengthens a cryptic splice site 422. They also suggest where the spliceosome may purposefully turn during biologically mediated alternative splicing. Cryptic splices 400 thus bring to light the hidden splice sites and exons in any gene, exposing the most likely locations that the splicing machinery will target under different biological conditions. Cryptic splices 400 also enable visualization and analysis of mutations from the sequence of a subject or cohort. In some embodiments, cryptic splices 400 may identify CSSs 412 in the genome of any organism including humans, animals, plants, bacteria, or fungi.

Cryptic splices 400 enable nested search boxes for the user to choose the genes having the highest number of cryptic sites, cryptic exons, highest cryptic site score, exon score, disease associated genes, and exceptional genes. Cryptic splices 400 create a repository containing information for genes in a genome such as exon details with the encoding domains, genomic position of the exons, transcript details, exon length, protein structure of the domain, real and cryptic splice donors, acceptors and exons, and enabling the display and analysis of any gene by a query. Cryptic splices 400 enable a search option for genes based on various parameters of cryptic sites, cryptic exons, cryptic splice site scores, and cryptic exon scores, and based on various gene panels such as disease panels, drug metabolizing gene (DMG) panels, the American College of Medical Genetics and Genomics (ACMG) gene panels, and other user given gene panels, and enable the display and analysis of any gene by a query. In some embodiments, a tab 461 may indicate the total number of cryptic sites (e.g., in a selected gene), and a tab 465 may indicate a total number of cryptic exons. A toggle switch 450A may turn on/off the illustration of mutations in the gene, as well.

In some embodiments, cryptic splices 400 create a landscape of real and cryptic splice scores across the entire gene by layering the scores of real splice sites, real exons, cryptic splice sites, and cryptic exons on the gene structure. In some embodiments, cryptic splices 400 predict the impact of a subject mutation on the action of the spliceosome, and determining where the spliceosome may erroneously make a mistake if a mutation weakens a real splice site or strengthens a cryptic splice site or vice versa by using the splice site scores. In some embodiments, cryptic splices 400 predict where the spliceosome may purposefully turn during biologically mediated alternative splicing, identifying the hidden splice sites and exons in any gene, and exposing the most likely locations that the splicing machinery will target under different biological and disease conditions. In some embodiments, cryptic splices 400 enable the use of tightly coupled navigation by interlinking different maps to provide analysis of a gene, protein, or other elements and features throughout the platform. In some embodiments, cryptic splices 400 perform analysis of subject mutations overlaid on the cryptic splice site patterns on the genes to correlate their associations with disease. Mutations from databases such as ClinVar, dbSNP, and COSMIC on cryptic splice site patterns on the genes may be correlated with disease. In some embodiments, cryptic splices 400 identifies real and cryptic splice sites using other relevant algorithms such as MaxEntScan, NNSplice, and Human splicing Finder throughout the gene sequence and genes in the genome and its application to subject and cohort genomics. In some embodiments, cryptic splices 400 discovers different sequence lengths for donor and acceptor splice sites that are more optimal when compared with 15/9 bases. Applying these lengths detects splice sites and their cryptic versions.

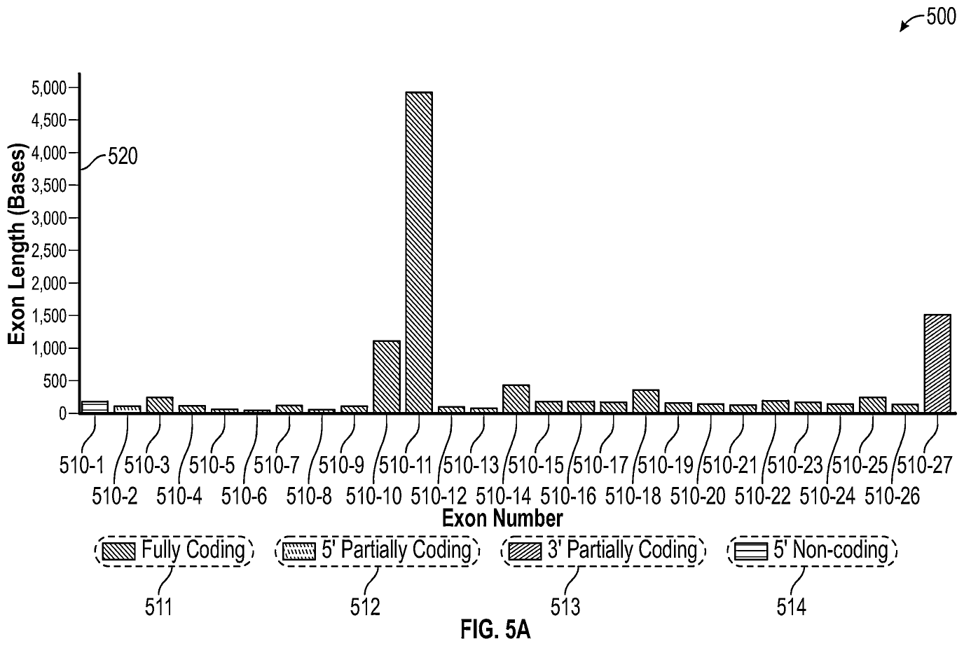

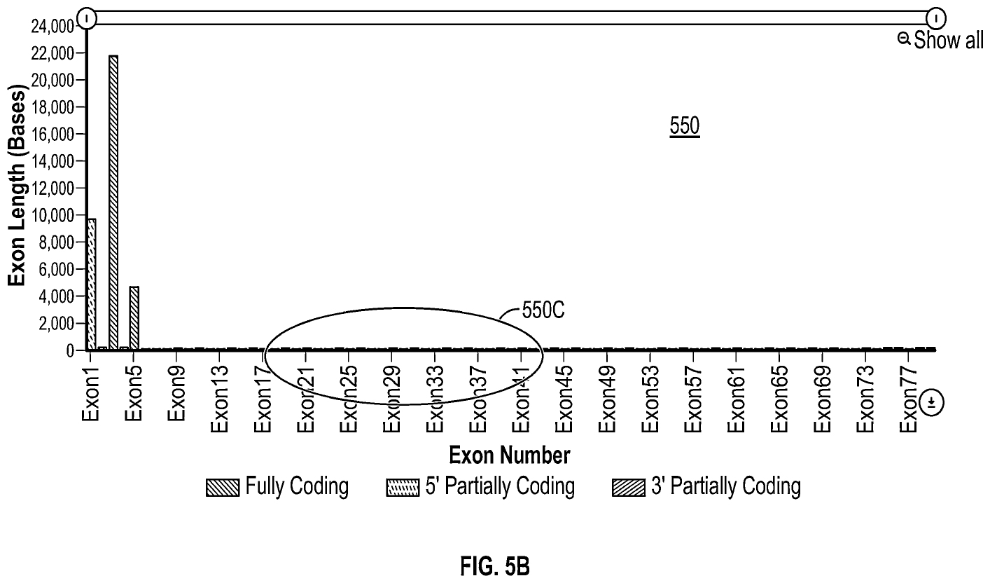

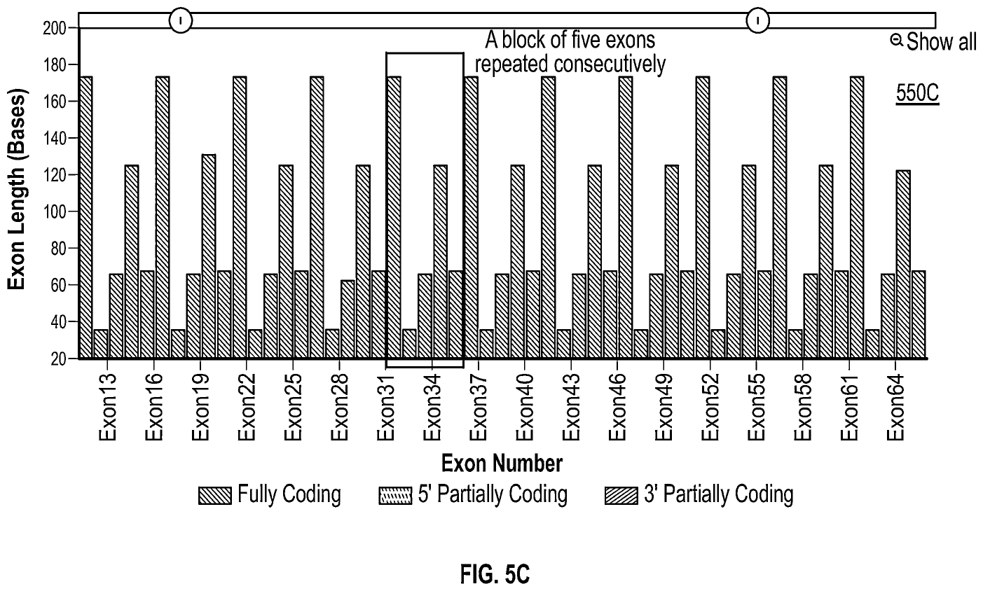

FIG. 5A illustrates an exon chart 500, according to embodiments disclosed herein. Exon chart 500 may be provided by an exon chart module (cf. exon chart module 260-3), as disclosed herein. Exon chart 500 is a map of the exon length 520 within human genes, containing multiple details for exons 510-1, 510-2, 510-3, 510-4, 510-5, 510-6, 510-7, 510-8, 510-9, 510-10, 510-11, 510-12, 510-13, 510-14, 510-15, 510-16, 510-17, 510-18, 510-19, 510-20, 510-21, 510-22, 510-23, 510-24, 510-25, 510-26, and 510-27 (hereinafter, collectively referred as “exons 510”). It displays the coding sequence (CDS) for each gene, and displays the lengths 520 of exons 510 and their associated splice scores in a graphical and sequence view. It additionally highlights any exon length repetition in each CDS, wherein multiple exons have the same length. Exon chart 500 also isolates exons 510 that have a highly outlying length compared to other exons 510 in a gene, and lists their splice scores as well as any cryptic splice sites contained within them. Exon chart 500 is thus a visual platform for analyzing the classification of exon lengths 520 and their accompanying splicing features, including unusual exon patterns in distinct genes. In some embodiments, exon chart 500 may provide further details for exons 510 (including the Shapiro & Senapathy score of the exon, acceptor and donor sites, and the exon and intron lengths) upon mouse hover by the user over the specific bar for a given exon 510.

Exon chart 500 maps exons in human genes as graphs of exon lengths 520 within each gene that creates visual bar charts of patterns such as unique exon length distribution, outlying exons, and length repetition. A detailed analysis of two additional features are also illustrated. The first is exon length repetition, wherein multiple exons in the gene have the same length. The second is highly outlying exon lengths (e.g., exon 510-11), in which one or more exons in the gene may be exceedingly longer than the others (e.g., exons 510-10 and 510-11). Each repeated or outlying exon length 520 can be further examined with full nucleotide string views and associated splice site scores.

The cause and effect of these features in human genes, which have important functions in a large number of diseases, are yet to be understood. As exon length 520 and intron length are closely associated with the splice sites and their sequences, exon chart 500 enables the understanding of these unusual features for selected human genes. By consolidating the lengths and splice site sequences of exons from the human genes, exon chart 500 permits the detection of exons 510 with unusual repeat lengths, outlying exon length patterns, and splicing patterns in distinct genes and their biological implications, and studies their associations with disease.

Exons 510 may be classified based on their coding features: 5′ non-coding sequences 514, 3′ non-coding sequences (not shown in FIG. 5), 5′ partially coding sequences 512, 3′ partially-coding sequences 513, and fully coding sequences 511. Various exons 510 present in a gene are characterized into multiple categories based on their length 520 to identify the exon length repetition property, highest exon lengths 520 to signify the “outliers” in the gene (exons with ≥3 times the length of average exons, e.g., exon 510-10 and 510-11), and the exception genes which contain no stop codon, in-frame stop codon, or selenocysteine codon sequences.

The splice acceptor and donor scores for each of the exon-intron junction sites are calculated using an algorithm (e.g., algorithm 250, such as the Shapiro & Senapathy and other relevant algorithms) to depict the biological probability and impact of the splicing event occurring at these sites. Cryptic splice sites are also determined based on the Shapiro & Senapathy and other relevant algorithms, within the selected exon sequence and their scores are tabulated. In addition, these real and cryptic splice sites are highlighted in the sequence view of the exons (cf. real splice sites 402 and CSSs 412).

The distribution of length of the exons in each of the transcripts of a gene is determined based on the CDS information including the number of coding exons, and length of each exon of all the transcripts of a gene. The length of the exons are plotted against each of the exons in a transcript and its distribution is identified. Furthermore, the length of exons that are repeating in a transcript is also identified and tabulated. Outlying exons are defined using various methods, including the exons with length greater than thrice the average exon length.

In addition, the exon chart module enables the user to search and query exon chart 500 to identify CDS length: Genes based on the coding sequence length can be searched ranging from 1-110,000 bases, which directly reflects the gene length. The exon chart module also enables the user to search and query exon chart 500 to identify exon length repetition: Genes that have exons of repetitive coding lengths can be chosen to determine the distribution of the exon lengths that are repeated. The exon chart module also enables the user to search and query exon chart 500 to identify outlying exon lengths: Genes that contain a significantly higher exon length when compared to the other exon lengths are termed as “outlying” exon lengths. Such genes with stark differences are identified by incorporating a rule that the outliers should be ≥3 times the length of average exons. The exon chart module also enables the user to search and query exon chart 500 to identify a gene: Defined based on the gene nomenclature, protein identifiers, clinical association, number of domains, and number of exons per domain, which are based on the user's preferences. The exon chart module also enables the user to search and query exon chart 500 to identify clinical association: The disease association of somatic cancer, germline cancer, non-cancer inherited disorders, industrial panels, ACMG gene panel, and DMG panel are enabled in the dropdown list. The exon chart module also enables the user to search and query exon chart 500 to identify exception genes: Genes that exhibit a rare characteristic exon behavior such as containing an in-frame stop codon, selenocysteine codon, or no stop codons at the end of the gene that are present in the sequence are shown.

In some embodiments, exon chart 500 may also identify the biomarker mutations for exons 510 from various data sources such as dbSNP, ClinVar, and COSMIC (cf. database 252), reported for different diseases. Accordingly, exon chart 500 may determine and illustrate a probability to develop a disease, for a given subject.

The exon chart module may also provide tabulated information based on exon chart 500, as illustrated in Table 2, below. For example, the exon chart module may identify exons 510 when an exon length is greater than or equal to three times an average exon length of a gene, based on exon chart 500. Accordingly, Table 2 indicates cryptic splice sites that occur within an outlying exon (e.g., exon 510-10 or 510-11). Table 2 displays the nucleotide string for the selected outlying exon, together with the real acceptor and donor as well as cryptic acceptors and donors are depicted in different colors on the sequence view.

TABLE II

| | | | | Total | |
|---|---|---|---|---|---|
| | | Real | Real | Cryptic | Total |
| Exon | | Accepter | Donor | Acceptor | Cryptic |
| Number | Exon length | Score | Score | Sites | Donor Sites |
| Exon 11 | 4,932 | 88.16 | 86.70 | 119 | 34 |

Select cryptic score threshold: 70

| Cryptic acceptor sites | | | Cryptic donor sites | | |
|---|---|---|---|---|---|
| Position | Sequence | Score | Position | Sequence | Score |
| 12 | TCTTCTGAAAGA | 72.58 | 232 | AAGGACAGT | 72.46 |
| 25 | GAAGCTGTTCACAGA | 70.68 | 470 | AATGTCAGA | 71.45 |
| 60 | TTGTCCTTAACTAGC | 74.15 | 487 | AAGGTAACA | 81.78 |
| 119 | TTCTAATAATACAGT | 78.06 | 549 | GATGTATGT | 74.04 |
| 130 | CAGTAATCTCTCAGG | 78.44 | 633 | AAGGTACAA | 71.57 |
| 146 | TCTTGATTATAAAGA | 76.08 | 889 | CAGGTGATA | 78.04 |
| 191 | ATTTATTACCCCAGA | 83.95 | 1,108 | GAGGTAGCT | 74.47 |
| 209 | TGATTCTCTGTCATG | 71.04 | 1,456 | GAAGTCAGT | 74.69 |

FIGS. 5B and 5C depict visualizations of an exon length distribution pattern 550 in the gene MUC16 in ExonChart Map.

FIG. 5B illustrates the exon length 550 having a long tail 550C indicative of a repeated pattern. The tail 550C includes the repetitive patterns of exon lengths having a marginal size.

FIG. 5C illustrates the tail end 550C of distribution pattern 550 repeated in a specific fashion. In tail 550C, exons of length 36 are repeated 15 times, exons of length 66 are repeated 10 times, and so on. Each block of 5 exons, with the lengths 173, 36, 66, 125, and 68, are repeated consecutively. It is to be noted that this gene MUC16 is an important cancer gene. This pattern is also connected with the repetition of a domain that is encoded by these exons as visualized in the Exon Splice map.

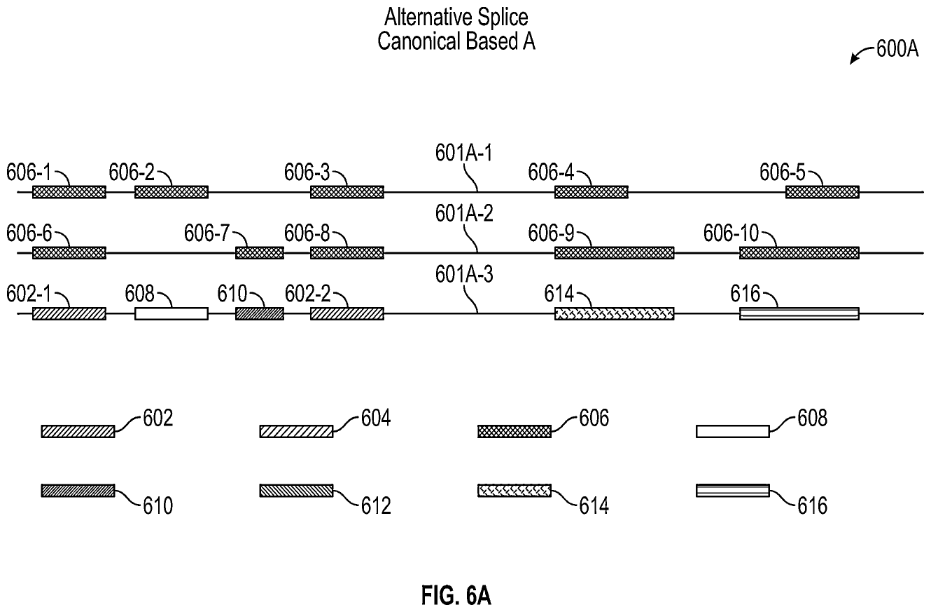

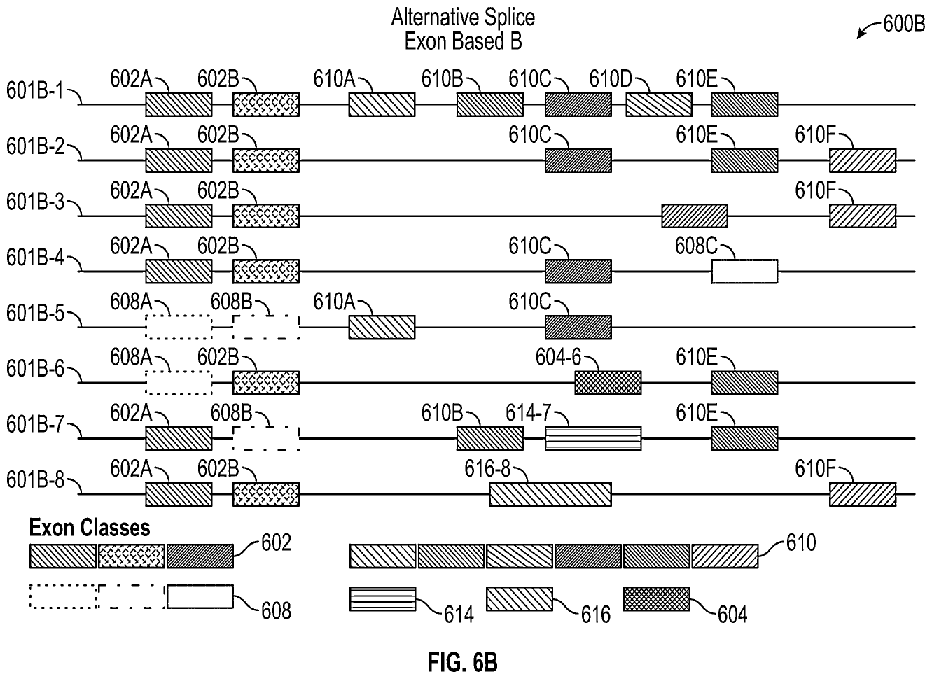

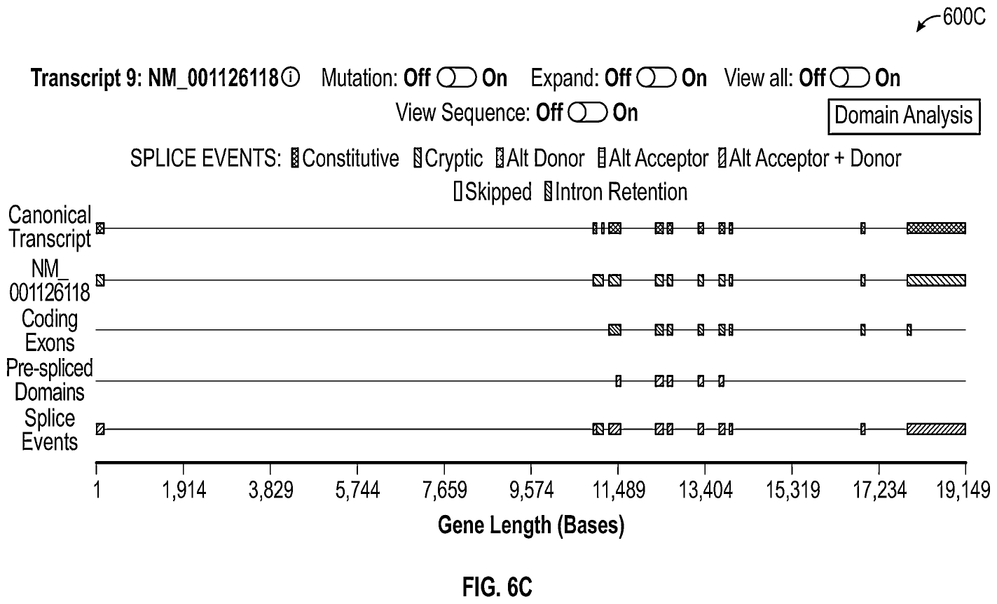

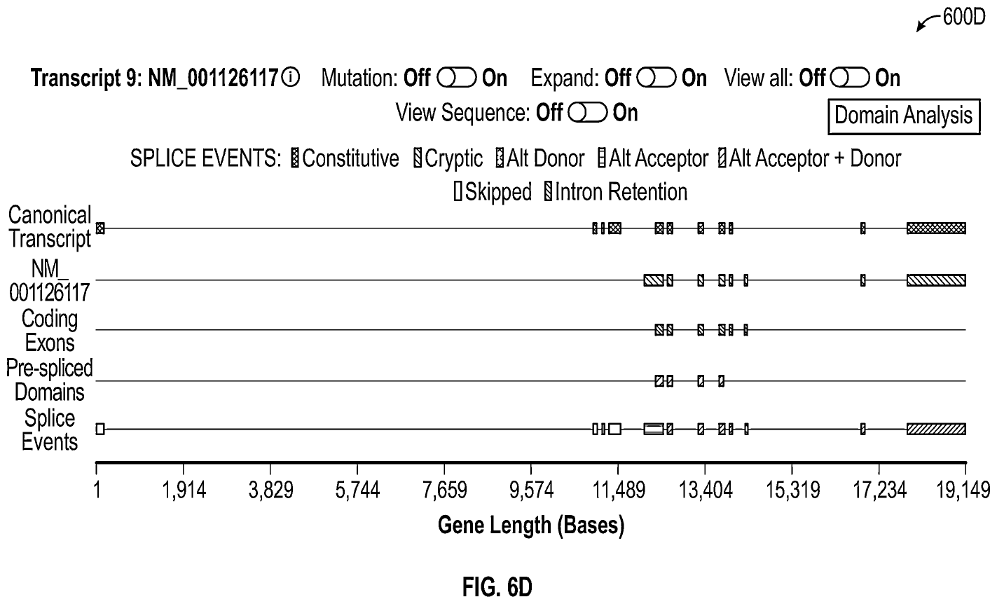

FIGS. 6A-6D illustrate exemplary embodiments of alternative splices 600A, 600B, 600C, and 600D (hereinafter, collectively referred to as “alternative splices 600”), as disclosed herein. In some embodiments, alternative splices 600 are provided by an alternative splice module as disclosed herein (e.g., alternative splice module 260-4). Alternative splices 600 illustrate alternative transcripts of a nucleotide string 601A-1, 601A-2, 601A-3, 601B-1, 601B-2, 601B-3, 601B-4, 601B-5, 601B-6, 601B-7, and 601B-8 (hereinafter, collectively referred to as “transcripts 601A,” “transcripts 601B,” and “canonical transcripts 601”). Alternative splice 600A illustrates a canonical-based splice event, and alternative splice 600B illustrates an exon-based splice event.

Transcripts 601A may be identified by the length of a CDS (e.g., the longest, or one of the longer CDSs) in a gene (length summation of all coding exons). In some embodiments, transcripts 601A are identified by a length of the corresponding mRNA (e.g., the longest, or one of the longer mRNAs) in a gene (length summation of all mRNA exons that includes coding and non-coding regions). In some embodiments, transcripts 601A may be identified by a number of mRNA exons (e.g., the highest, or one of the higher numbers) in the gene. In some embodiments, transcripts 601A may be identified by a number of coding exons (e.g., the highest, or one of the higher numbers) in the gene. When there are more than one transcripts 601A with the same values in a selected method from above, a canonical transcript 601 may be selected when it is annotated as “canonical” in a third party database (e.g., database 252, such as UniProt database). When the “canonical status” is not available in a third party database, the alternative splice module randomly assigns the “canonical” status to one of the two or more transcripts that satisfy the above criteria.

FIG. 6A illustrates alternative splice 600A including constitutive events 602-1 and 602-2 (hereinafter, collectively referred to as “constitutive events 602”), wherein the same exons are present as in the canonical transcript; alternative acceptor and alternative donor events 604, wherein both start and end exon positions of the present transcript is different from the constitutive exon. Alternative splice 600A may also include mRNA exons 606-1, 606-2, 606-3, 606-4, 606-5, 606-6, 606-7, 606-8, 606-9, and 606-10 (hereinafter, collectively referred to as “mRNA exons 606”); skipped events 608, wherein skipped exons are the constitutive exons that are not present in the transcript of interest; and cryptic events 610, wherein exons occur newly in the present transcript as compared to the constitutive exons. Alternative splice 600A may also include intron retention events 612, wherein an exon start position of the present transcript matches with one exon in canonical and exon end position match with the end of another exon. The intronic region between these two exons is marked as intron retention event 612. Alternative splice 600A may also include an alternative donor event 614, wherein an exon start position of the present transcript is the same as that of constitutive exons but the exon end position is different; and an alternative acceptor event 616, wherein the exon end position of the present transcript is the same as that of constitutive exons but the exon start position is different.

FIG. 6B illustrates alternative splice 600B including an ‘Exon based’ selection, wherein the canonical transcript includes exons identified by number or count of exons across the transcript for a given gene. The exons which occur more than or equal to 50% of the total number of transcripts, are identified as constitutive exons 602. When multiple constitutive exons 602 exist, an alternative splicing event is defined with respect to any exon that shares a start or end position with the exon in the selected transcript. In some embodiments, constitutive exon 602 is selected from exons that are present in more than 50% of the number of transcripts in a gene and are classified as constitutive exons. To illustrate the above, transcripts 601B illustrate constitutive exon 602A in the 5′ end of transcripts 601B-1, 601B-2, 601B-3, 601B-4, 601B-7, and 601B-8, followed by constitutive exon 602B in transcripts 601B-1, 601B-2, 601B-3, 601B-4, 601B-6, and 601B-8. An alternative donor and acceptor event 604 occurs when both the donor and acceptor splice sites of an alternative exon are different from that of the constitutive exon (e.g., alternative donor and acceptor event 604-6 in transcript 601B-6). Accordingly, the alternative exon is assigned with both alternative donor and alternative acceptor sites. A skipped exon event 608 occurs when any of the constitutive exons 602 are missing in a transcript, and these missing exons are classified as skipped exons in that transcript. To illustrate this, skipped exon events 608A indicate the skipping of constitutive exon 602A in transcripts 601B-5 and 601B-6; skipped exon events 608B indicate the skipping of constitutive exon 602B in transcript 601B-5 and 601B-7; and skipped exon event 608C indicates the skipping of alternative exon 610E in transcript 601B-4.

A cryptic exon event 610 occurs when an exon found in less than 50% of the transcripts in a gene are classified as a cryptic exon. To illustrate the above, alternative exon 610A appears in transcripts 601B-1 and 601B-5, only; alternative exon 610B appears in transcripts 601B-1 and 601B-7; alternative exon 610C appears in transcripts 601B-1, 601B-2, 601B-4, and 601B-5; alternative exon 610D appears in transcript 601B-1; alternative exon 610E appears in transcripts 601B-1, 601B-2, 601B-6, and 601B-7; and alternative exon 610F appears in transcripts 601B-2, 601B-3, and 601B-8. An alternative donor event 614 occurs when an exon has a donor splice site different from that of constitutive exon 602, and that different splice site is classified as an alternative donor site (as indicated by alternative exon 614-7 in transcript 601B-7). An alternative acceptor event 616 occurs when an exon has an acceptor splice site different from that of constitutive exon 602, then the acceptor splice site is classified as an alternative acceptor site (as indicated by alternative exon 616-8 in transcript 601B-8).

FIG. 6C and FIG. 6D illustrate alternative splices 600C and 600D in alternative splicing of isoforms of the gene TP53, showing the types of exons such as constitutive, cryptic, and altered acceptor and/or donor in different color codes.

In some embodiments, alternative splices 600 may display the exons of canonical transcripts 601. In some embodiments, alternative splices 600 may display the exons of currently selected transcripts. In some embodiments, alternative splices 600 display coding exons of a current transcript. In some embodiments, alternative splices 600 display available domains for a particular transcript. In some embodiments, alternative splices 600 display splice events of exons in the current transcript by comparing with canonical transcripts 601. In some embodiments, alternative splices 600 also illustrate mutations from individual subjects and from cohorts of subjects for a selected gene. This visualization aids in the analysis of their disease associations. In addition, domain analysis sections displaying the domains coded by the exons of alternatively spliced events are also enabled.

Based on the various search options including genes, number of transcripts, splice events, and clinical associations, the alternative splice view of the selected gene is visualized. The constitutive exons can be selected based on the two methods (Canonical and Exons). In the ‘Canonical Based’ selection, the “longest CDS” option displays the canonical transcript, showing the coding exons, non-coding exons, pre-spliced domains, and the alternative splicing events respective to the canonical transcript. In the ‘Exon based’ selection, the alternatively spliced exons are classified and shown as constitutive, cryptic, alt donor, alt acceptor, alt acceptor+donor, exon skipping, and Intron retention events for the selected gene and transcript.

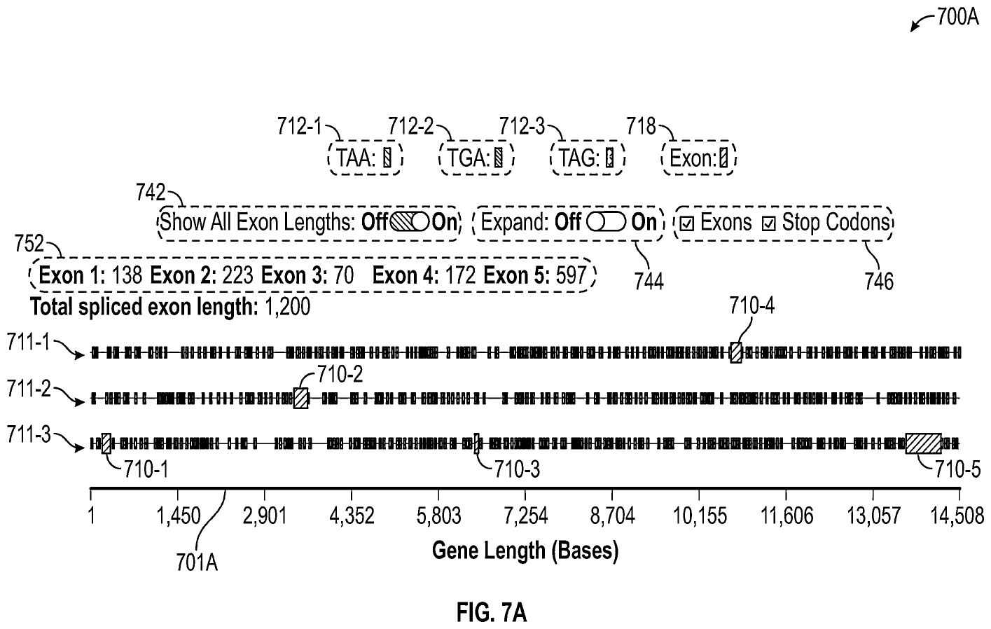

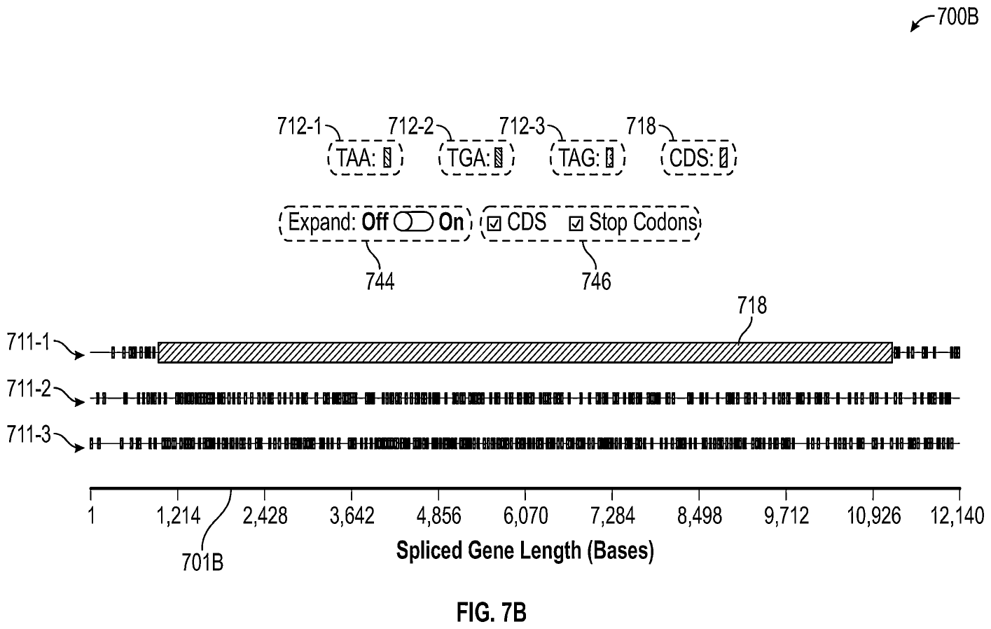

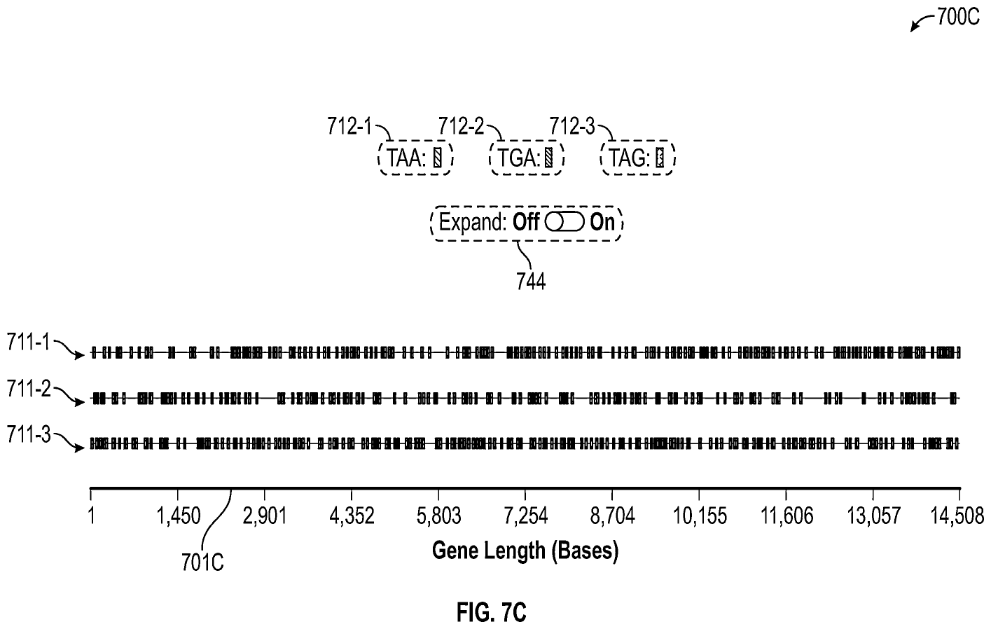

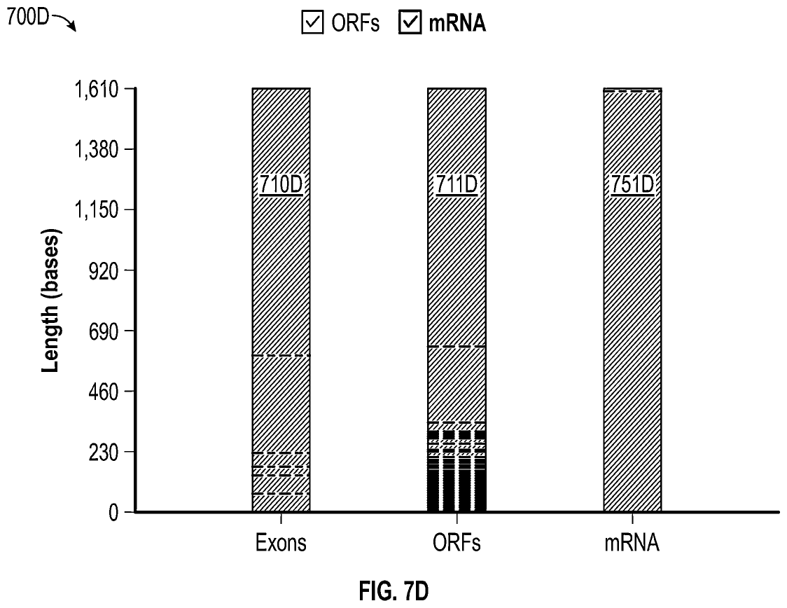

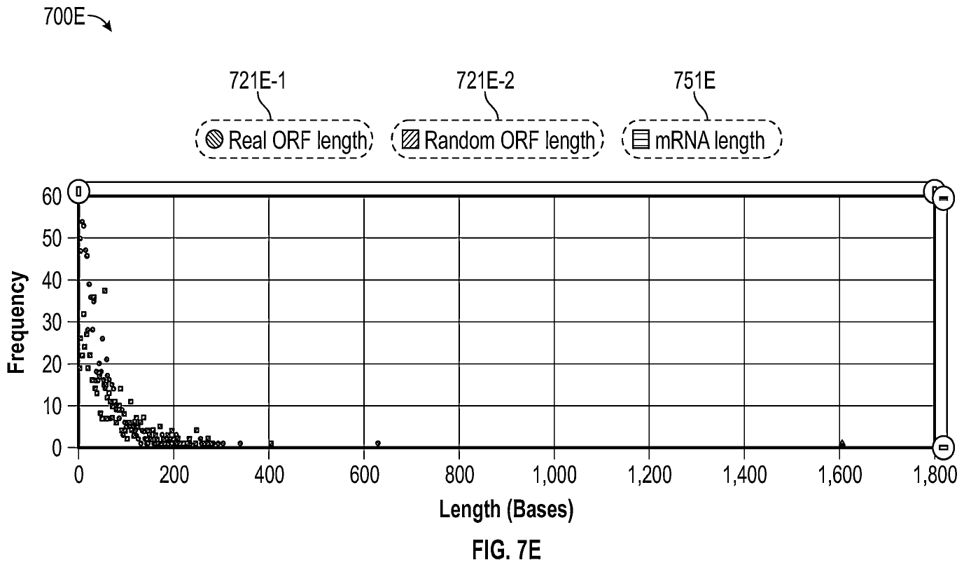

FIGS. 7A-7E illustrate exemplary embodiments of exon frames 700A, 700B, 700C, 700D, and 700E (hereinafter, collectively referred to as “exon frames 700”), as disclosed herein. Exon frames 700 may be provided by an exon frame module as disclosed herein (cf. exon frame module 260-5). Exon frames 700 include maps coding exons in a gene and designate their reading frame in a transcript indicated as 711-1, 711-2, and 711-3 (hereinafter, collectively referred to as “reading frames 711”). Exon frames 700 include a picture of exons 710-1, 710-2, 710-3, 710-4, and 710-5 (hereinafter, collectively referred to as “exons 710”) in reading frames 711. In some embodiments, coding exons 710 are placed in the reading frame in which they occur before RNA splicing. Exon frames 700 include an image of the entire split gene, with exons 710, introns, and stop codons 712-1 (TAA), 712-2 (TGA), and 712-3 (TAG, hereinafter collectively referred to as “stop codons 712”) that occur within each frame. In some embodiments, stop codons 712 are scanned in a sliding window method against the nucleotide strings and placed in the respective reading frames. Exon frames 700 streamline the detection of atypical gene patterns, such as long exons (cf. exon 710-5), long open reading frames without annotated exons, or short introns. In some embodiments, exons 710 are displayed in a single reading frame of the gene along with their splice sites and scores.

Based on the available search criteria, a transcript for the selected gene is displayed with reading frames 711 before and after the splicing process.

FIG. 7A illustrates exon frame 700A, which presents a transcript in reading frames 711 with stop codons 712, and coding exons 710, before splicing.

FIG. 7B illustrates exon frame 700B, which presents the transcript in reading frames 711 with stop codons 712, and the longest CDS 718 (spliced exon), after splicing.

FIG. 7C illustrates exon frame 700C, which displays the distribution of stop codons 712 in a randomly generated nucleotide string. In some embodiments, stop codons 712 are marked in different colors. The exons are plotted in a different color (e.g., as rectangles 718).

The reading frame, exon number, position, length, and several other details 752 are displayed upon mouse hovering the coding, non-coding, or partially coding exons 710. The selected gene is also represented in a single reading frame with exons and stop codons in the same pattern of “Before splicing” and “After splicing” views (e.g., exon frames 700A and 700B, respectively). Exon frames 700A, 700B, and 700C may offer one or more graphic interface features such as a toggle 742 to select the display of all the exon length, a toggle 744 to expand the exon display, and a selection tab 746 to include either exons 710, stop codons 712, or both in the display.

FIG. 7D illustrates exon frame 700D including, for each gene, a unique “ExCode” 710D, which portrays exon lengths as lines in a bar, a code 711D portraying reading frame lengths, or a code 751D which portrays mRNA length. Codes 710D, 711D, and 751D uniquely identify a gene, akin to a barcode identifier. Exon frames 700 thus enable a clear view into the special features of reading frames 711, exons 710, introns, and their correlations that exemplify eukaryotic split genes.

FIG. 7E illustrates exon frame 700E that illustrates the distribution of ORFs length in a randomly generated sequence with the same gene length to compare their frequency with the ORF length distribution in real sequence. An amchart representing an overlapping curve is shown with the distribution of the length of ORFs in random sequence and real sequence. Frequency is labeled in Y-axis and the length of ORFs in X-axis. The length of ORFs and its frequency are reported upon mouse hover.

Nucleotide strings 701A, 701B, and 701C (hereinafter, collectively referred to as “nucleotide strings 701”) are scanned for the various stop codons 712 and labeled in each reading frame 711, e.g., indicated by color—red blue green—and the like). The coding exons are spliced together, which is a form of RNA processing. Exon frame 700B displays the longest CDS 718 that occurs after splicing in the single reading frame of the gene along with their splice sites and scores. Exon frames 700 also illustrate, in a randomly generated nucleotide string, detection of any long introns and exons, short introns, unusual distribution of stop codons, and long open reading frames. In some embodiments, exon frames 700 also enable a clear view of the sequence features that exemplify eukaryotic split genes.

In some embodiments, reading frames 711 are computed and plotted by dividing the length of exons as follows: i) RF (Reading Frame)=((Exon Start on gene−1) % 3) (or); ii) RF=(Exon Start on gene−Previous Exon End on gene+Reading Frame of Previous Exon+1) % 3; iii) If the calculated result is 0, the exon is placed at reading frame 711-1; iv) If the calculated result is 1, the exon is placed at reading frame 711-2; v) If the calculated result is 2, the exon is placed at reading frame 711-3.

The length of the spliced exons (e.g., CDS 718) and spliced string are calculated as follows: i) Spliced Length=(First Exon Start−1)+(Sum of the Length of all exons)+(Gene End−Last Exon end); ii) Spliced String=Concatenate Sequence before first exon, all exons sequences, and sequence after last exon.

Stop codons 712 are scanned against the spliced string in a sliding window against and plotted in reading frames 711. The reading frame for the spliced exons (e.g., CDS 718) is calculated as: ((First exon start−1) % 3). In addition, a random nucleotide string with the same gene length is generated.

In some embodiments, the lengths of exons, ORFs 711, and mRNA are analyzed in ExCode. The length of all exons, ORFs, and mRNA are marked on a graphical illustration for all the selected transcripts. Stop codons 712 and ORFs that are available are marked for reading frames 711. The length of ORFs and Exon lengths are compared creating exon identifier 710D for the transcripts. The distance between two stop codons 712 is thus analyzed and other possible stop codons are mapped while expecting them not to fall inside coding exons 710.

Exon frame 700E illustrates a distribution of ORF lengths for a given gene. The original gene sequence and the randomly generated gene sequence are sourced and ORFs are plotted as real ORF lengths 721E-1, random ORF length 721E-2 (hereinafter, collectively referred to as “ORF lengths 721E”), or mRNA length 751E. The ORFs are identified as sequences between two consecutive stop codons and their lengths 721E are determined accordingly. ORF lengths 721E and their frequencies are collected in reading frames 711 and plotted to analyze the distribution of ORF lengths 721E. In some embodiments, tabular information may be provided to the user by a mouse hover over exon frames 700 (cf. Table III, below).

TABLE III

| Exon | Acceptor 3’ | Donor 5’ |
|---|---|---|
| Number | splice signal | splice signal |
| 1 | NA | CAG\|GTGAGC |
| 2 | CTCTTCTTTTTCAG\|A | GTG\|GTAAGC |
| 3 | TTCTTGCTCTTCAG\|G | AAG\|GTAGGC |
| 4 | TCTTGTCCCCGCAG\|C | AAG\|GTACGT |
| 5 | TTCCCTTCCCACAG\|G | AAG\|GTATTT |
| 6 | TTAATCTTTTACAG\|A | CAG\|GTAAAG |
| 7 | CTTTTGGTTTTCAG\|G | GAG\|GTACTG |
| 8 | CATTCTAATCTAG\|G | CAG\|GTACGT |
| 9 | TCTATGAAAGCAG\|G | CAG\|GTGAAA |
| 10 | ATCATTCTTTGCAG\|A | CGA\|GTAAGT |

In Table 3, stop codons that occur at the −3 position of the acceptor and the +2 position of the donor are highlighted (e.g., in ‘red’).

In some embodiments, exon frames 700 may include nested dropdown lists to select different parameters to display information and details such as various transcripts of a selected gene sourced from a third party database (e.g., NCBI database). The user can study exon frames 700 for genes with varying length of the coding sequence ranging from 1-110,000 bases in the list, or more. For each range, exon frames 700 provide the corresponding transcripts. In some embodiments, exon frames 700 include a dropdown list to enable genes having from 1-400 exons, or more, from which the user can select genes and study the pattern of exon reading frames and splicing events. For each range, the corresponding transcripts are also provided. In some embodiments, exon frames 700 may enable selecting genes according to their length, e.g., ranging from 1-3,000,000 bases, or more. Upon selecting the range of length, the corresponding genes are listed for which the user can study the patterns of exon frames and splicing events.

In some embodiments, exon frames 700 enable the user to select genes based on a clinical association. Accordingly, the user may select genes from panels for different disease categories such as Somatic cancers, Germline cancers, Non-cancer Inherited disorders, Industrial panels, ACMG, and DMG panels, to visualize the exon frame for the selected genes.

In some embodiments, exon frames 700 enable the user to select exception genes that: (i) Contains an in-frame stop codon: Genes having stop codons (TAA, TGA, TAG) inside the reading frame; (ii) Contain a selenocysteine: an unusual stop codon (mostly TGA) in the coding sequence, and (iii) Contain no stop codons 712.

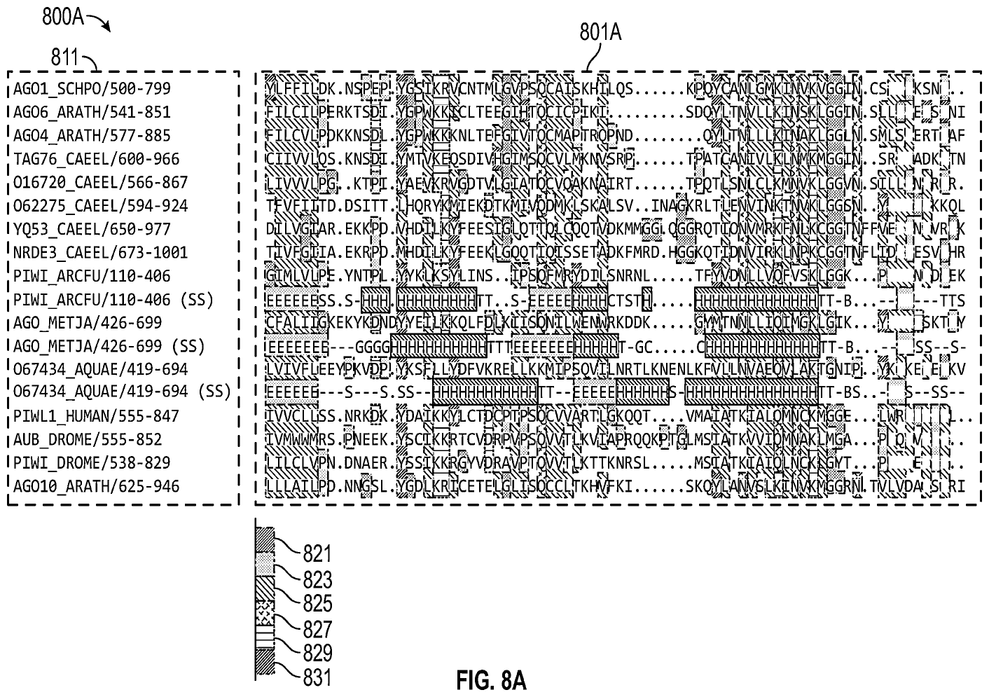

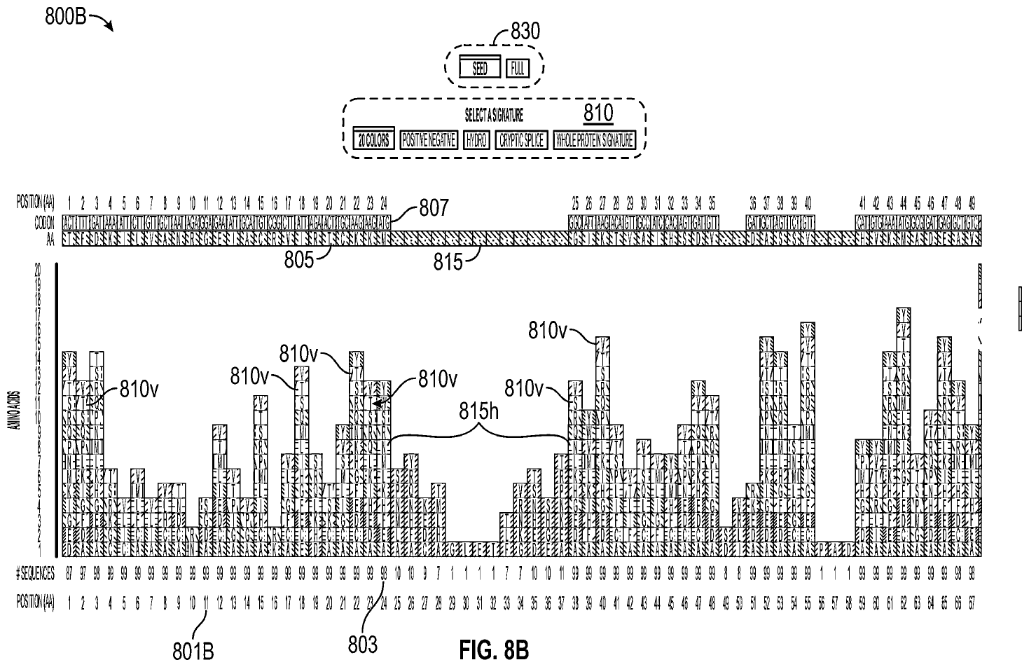

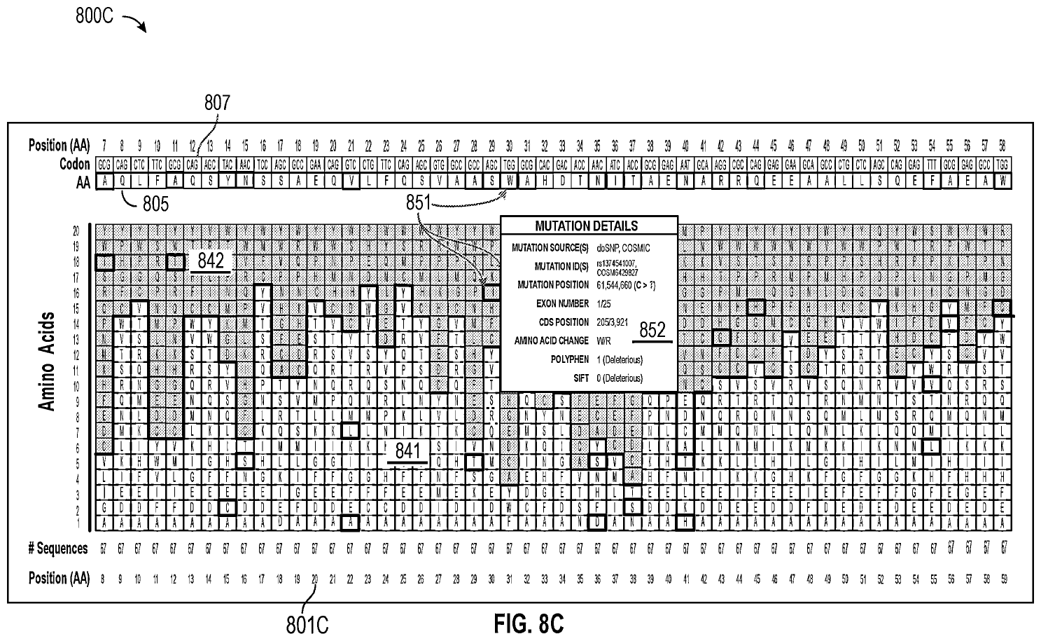

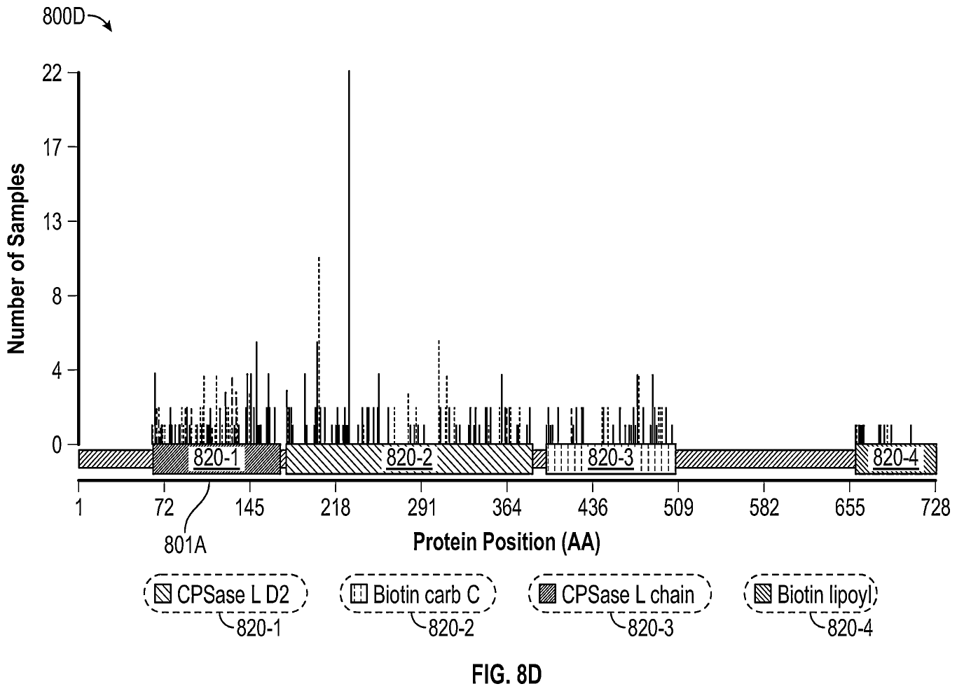

FIGS. 8A-8D illustrate exemplary embodiments with protein charts 800A, 800B, 800C, and 800D of a protein signature (hereinafter, collectively referred to as “protein charts 800”), according to embodiments disclosed herein. Protein charts 800 are associated with amino acid strings 801A, 801B, and 801C (hereinafter, collectively referred to as “amino acid strings 801”).

Cryptic splice sites within the domain coding exons are determined based on the S&S and other relevant algorithms and plotted on the exons above the domain signatures. Cryptic splice sites are shown based on the selected score threshold as red boxes for acceptors and green boxes for donors and highlighted their corresponding sequence in the codon sequence below the exons. Alternative splicing events are determined by comparing the exons from the canonical transcripts in two different ways. 1) Exons that are present in 50% or more of transcripts are defined as constitutive, and alternative splicing events in the signature are shown with respect to these exons. 2) The transcript with the highest number of exons are defined as canonical, and alternative splicing events in the signature are shown with respect to this transcript. Any alternative splicing events that occur based on the occurrence of exons or relative to the canonical transcript are highlighted in these exons. Any skipped exons (or portions of exons) in the selected transcript are shown in black, and any added exons (or portions of exons) are shown in blue. The AA signature for any skipped or added exon region is shown below the corresponding exon positions.

The splice sites identified in the coding exons are predicted by employing the Shapiro-Senapathy and other relevant algorithms. Based on the variable threshold score range (e.g., 50 to 100) chosen, the splice sites having scores within the selected range are visualized on the plot. The cryptic and real splice sites are depicted in various color codes and overlain on the CDS plot and the signatures as well. The domain coding regions of the coding exons are aligned to the human domain sequence above the domain signature and the cryptic splice sites falling within this region are displayed along with their scores and splice sequences.

Genes can be searched based on various search criteria. Based on the selection criteria, the gene and transcript information are displayed. Information like gene name, chromosome number, gene ID, strand, protein ID, protein length and number of exons are displayed along with details on gene ontology and phenotype on clicking the “Gene Info” button available in the information strip. ProtSig is divided into three different sections: Protein overview, Cryptic splice sites, and Variant density.

A protein overview section visualizes the coding exon of the selected gene along with the domain information overlaid as colored lines. By default, the compact view of the module is displayed. The expanded view can be displayed by switching the expanded view toggle “ON” at the top of the plot. The mutations curated for the selected gene can be depicted on the plot by configuring the mutations toggle. The databases used in curating the mutations for the selected gene are: dbSNP, ClinVar, and COSMIC. The mutation details fetched from the respective databases are displayed on hover of a particular mutation. The mutations from a patient exhibiting a disease can also be overlaid on any of the protein signatures and on the Positive-Negative protein signatures.

The cryptic splice section aids in visualizing exons that encode the domain, along with their codon and AA sequences. The cryptic splice sites within these domain coding exons are determined using the S&S and other relevant algorithms and are marked on the exons based on the selected score threshold from the dropdown. The cryptic acceptors are represented in red color and the cryptic donors are shown in green color. The score and sequence of the cryptic splice sites are displayed on mouse hover.

The alternative splicing signature depicts the signature of the domain region that is skipped or added during the alternative splicing process. Alternative splicing events that occur relative to the canonical transcript are highlighted in these exons. Skipped exons (or portions of exons) in the selected transcript are shown in black, and any added exons (or portions of exons) are shown in blue. The AA signature for skipped or added exon region is shown below the corresponding exon positions.

Protein charts 800 aid in visualizing the data from both the seed and full alignment for a selected domain ID. It visualizes the number of non-redundant AAs produced from the multiple sequence alignment in each position in the transcript. The number of amino acids and the domain position is displayed on mouse hover of the peaks in the signature plot.

The cryptic splice sites section aids in visualizing the coding exon of the selected gene overlaid with splice sites based on the selected threshold score. The different types of splice sites are color coded: cryptic acceptors in red, cryptic donors in green, and real sites in blue. The scores calculated by employing the SS algorithm are depicted above each site for donors and below each site for acceptors. The site details like start position, end position, sequence, and score can be displayed by hovering over the marking on the coding exons.

Protein charts 800 allow visualizing the altered amino acids falling within the set of allowed amino acids or the counterpart. The unique set of amino acids for each position of the domain from the seed or full alignment files are depicted as stacks in the green region, whereas the amino acids other than the allowed set are depicted in the red region, showing the allowed and non-allowed amino acids. It is thought that if the altered amino acids fall within the allowed set, the function of the domain is not affected. However, the domain's function is greatly stirred when the altered amino acid is not accounted for in the allowed set.

FIG. 8A illustrates protein chart 800A, including multiple sequence alignments of amino acid strings 801A for a protein coded in diverse genomes having identifiers 811. For each position set of “allowed” amino acids at each sequence position, each are generated in ProtSig (using an algorithm described below), creating a signature of potential amino acid substitutions across the domain. These signatures are color-coded based on multiple distinct parameters, such as the degree to which the amino acids are hydrophobic or hydrophilic, and whether they correspond to a region that is alternatively spliced. For example, a Glycine AA may be indicated by code 821, and a Proline AA may be indicated by code 823. A code 825 may indicate a small or hydrophobic AA (e.g., C, A, V, L, I, M, F. W), a code 827 may indicate Hydroxyl or amine amino acid groups (e.g., S, T, N, Q), a code 829 may indicate charged amino acids (e.g., D, E, R, K), and a code 831 may indicate a Histidine or Tyrosine amino acid (e.g., H, Y). For every unique identifier 811, an alignment section 801A is available in the seed/full file. Alignment sections 801A are parsed to identify the unique amino acids at each sequence position along with the gaps and are considered as amino acid stacks. A signature for the selected protein domain is created by these stacks present in each position of the sequence alignment in strings 801A. The alignment section including all strings 801A is parsed such that at each position, the unique AAs from the alignment are taken including “.” (gaps) and are considered as stacks. For example, the stack for the ninth position is “.RKEY” (e.g., as can be verified by looking at all amino acid strings 801A down from the 9th letter—with many gene variants missing-). The stacks in each of the positions in the alignment of a list of identifiers 811 forms the signature of the domain.

FIG. 8B includes protein chart 800B, including a signature impression of the amino acid substitutions that likely maintain the structure and function of a given protein region, and helps bridge the divide between protein structure, function, splicing, mutations, and disease. Accordingly, the protein signature module converts the alignments in protein chart 800A into a signature in protein chart 800B by identifying the variable amino acids and avoiding the redundant amino acids at each position in amino acid string 801B. The signature in protein chart 800B is represented in graphical form as stacks of AAs for each position. The stacks whose positions had gaps (“.”) in more than 50% of the total number of sequences in the alignment are shown in grey boxes. A selection tab 830 allows the user to switch between seed and full alignment.

From the alignment, the human domain sequence alone is taken as such and represented in blue boxes 805 above the signature plot. This also includes gaps 815. The secondary structure information of the amino acids 807 is also provided in the signature of protein chart 800B. The number of sequences considered in the alignment 803 are also provided along with the number of gaps and number of amino acids strings 801B other than gaps in each position of the domain signature in the signature plot of protein chart 800B. In some embodiments, protein chart 800B visualizes the number of non-redundant AAs produced from the multiple sequence alignment in each position in the transcript. The number of amino acids and the domain position is displayed on mouse hover of the peaks in the signature plot.

In some embodiments, a variable amino acid 810v is displayed in protein chart 800B when it occurs at least at greater than a specific fraction (e.g., 50%) of the aligned positions in protein chart 800A. Protein signature module identifies different amino acids at each position and includes them as the variable or allowed amino acid 810v at that position. The set of variable amino acids 810v that does not alter the protein functionality is referred to as an “allowable set.” Accordingly, for each position, any one of the 21 different available amino acids that are not in the allowable set belong to a “non-allowable set.” The specific modality for presentation of the chart may be selected by the user via a select signature tab 810. For example, protein chart 800B indicates a protein signature indicating stacks of allowed amino acids represented in 20 colors (one color for each amino acid). Typically, when a mutation replaces an amino acid in the allowable set with an amino acid in the non-allowable set, the result is a dysfunctional protein, or a protein having a deleterious functionality. Any position 805 with “.” or “−” in the alignment indicating a gap is taken into account, whereby a position with a particular frequency (e.g., >50%) of dots is defined as a grey region 815 in the signature. Grey region 815 includes the least significant on the allowed amino acid set as it has gaps or dots predominantly (e.g., >50%). Protein chart 800B includes positions 815 in the human domain sequence that contain a gap, but the corresponding signature 815h at those positions are not grey regions, indicating that there are more than 50% of amino acids at that position in the alignment. In addition, there are positions in the human domain sequence containing amino acids but the corresponding signature is a grey region, meaning that there are more than 50% of gaps in that position in the alignment but the human sequence has an amino acid. Variable amino acids 810v in the protein chart 800B play an important role in determining the pathogenicity of variants and their mutational impact and clinical significance in terms of protein functionality. Accordingly, the protein signature module defines a deleterious mutation as one changing an allowed amino acid to a non-allowed amino acid in the signature of protein chart 800B. Accordingly, protein chart 800B enables identifying pathogenic mutations when the resulting amino acid falls in the non-allowed set, and mutations resulting in amino acids falling in the allowed set may be benign.

FIG. 8C includes protein chart 800C, which lists allowable sets 841 and non-allowable sets 842 of amino acids for each amino acid position 801C in a selected protein when the user selects a positive/negative display in tab 810 (cf. protein chart 800B). In some embodiments, protein chart 800C may validate and verify a designation of deleterious mutations 851 from third party databases (e.g., database 252, including dbSNP or other databases). Mutations 851 from a subject exhibiting a disease can also be overlaid on any of the protein signatures and on protein chart 800C. The mutated amino acids may be highlighted in a colored (e.g., ‘purple’) box in the signature plot. The mutation details are displayed on hover of the purple box along with PolyPhen and SIFT information. Moreover, protein chart 800C may provide a mutation information 852 when mouse hovering over the amino acid at position 30 (W—Tryptophan) leads to three deleterious mutations (R, L, and C) that fall in the non-allowed region (red), confirming that the algorithm based on this concept is valid. In some embodiments, information 852 for a selected mutation may be provided by a third party database (e.g., database 252, including the COSMIC database) along with the number/frequency of samples (subjects) having those mutations in their studies. Furthermore, if a deleterious mutation assessed by current methods falls within the green region itself, this may indicate that the designation of the mutation as “deleterious” may be erroneous. Accordingly, protein chart 800C may provide a basis for testing whether a given variant is deleterious or not (benign). In some embodiments, color codes may be used in protein chart 800C to contrast allowable sets 841 (‘green’) with non-allowed sets 842 (‘red,’ ‘pink,’ or ‘salmon’). In some embodiments, protein charts 800 include a color-coding of the amino acids based on their hydropathy index values from blue to red from hydrophilic to hydrophobic. Blue boxes 805 and amino acids 807 are as described in chart 800B.

FIG. 8D illustrates protein chart 800D including the frequency/density of different variants to visualize the number of samples for each variant in each protein domain position (e.g., as curated by the COSMIC database). A color code may be used for graphical aid. For example, positions in amino acid string 801A with a single variant are represented in red, and positions with more than one variant are depicted as follows: two variants—blue, three variants—green, four variants—yellow, and more than four—magenta. The mutation position, ID, and amino acid change along with the number of samples are displayed on mouse hover of the peaks depicted in the plot. Protein chart 800D illustrates mutation frequencies associated with domains 820-1 (CPSase L D2), 820-2 (Biotin carb C), 820-3 (CPSase L chain), and 820-4 (Biotin lipoyl) within the selected protein (hereinafter, collectively referred to as “protein domains 820”). The number of samples for each of the variants at a specific position in amino acid string 801A of a domain 820 may be retrieved from a third party database (e.g., database 252 including the COSMIC database). Accordingly, protein chart 800D provides a visual indication of the number of variants at a specific position in a given domain 820 based on the different color codes.

The protein signature module enables the user to explore protein signatures via protein charts 800 by providing the ability to search over one or more databases based on different criteria, such as gene based criteria, wherein the protein signature can be visualized by selecting the appropriate gene name along with its transcript ID. Or based on the number of domains and families, wherein a dropdown menu including the number of domains and families includes values ranging from 1 to 304, or even more. On selecting a number from the dropdown, the genes with corresponding number of domains and families are listed and the very first gene is visualized as default. Protein charts 800 may also allow the user to search protein signatures based on an average value of amino acid substitutions. Accordingly, protein charts 800 may include a dropdown menu including, for example: 20-16, 15-11, 10-6, and 5-1 amino acid substitutions. Protein charts 800 may also allow the user to search protein signatures based on a Pfam ID. Accordingly, a protein signature may be visualized by selecting an appropriate Pfam ID along with the gene name and transcript ID. Protein charts 800 may also allow the user to search protein signatures based on the alignment type. Accordingly, the protein signature can be visualized by selecting the appropriate alignment type 830 (seed/full) along with the gene name and transcript ID. Protein charts 800 may include an alignment type dropdown menu with the following values: only seed, only full, and seed and full. Protein charts 800 may also allow the user to search protein signatures based on a clinical association. Accordingly, protein charts 800 enable the user to select a disease category such as Germline cancer, Somatic cancer, ACMG panel, inherited disorder, Industrial panel, and DMG panel along with the disease name. The protein signature can be displayed for the selected gene based on the clinical association. Protein charts 800 may also allow the user to search protein signatures based on exception genes. Accordingly, protein charts 800 enable a user to visualize the protein signature of genes falling under the following criteria: (i) Contains an in-frame stop codon: Displays genes having stop codons in the reading frame; (ii) Contains a selenocysteine: Displays genes having selenocysteine (unusual amino acid); and (iii) Contains no stop codon: Displays genes having no stop codon at the end of CDS.

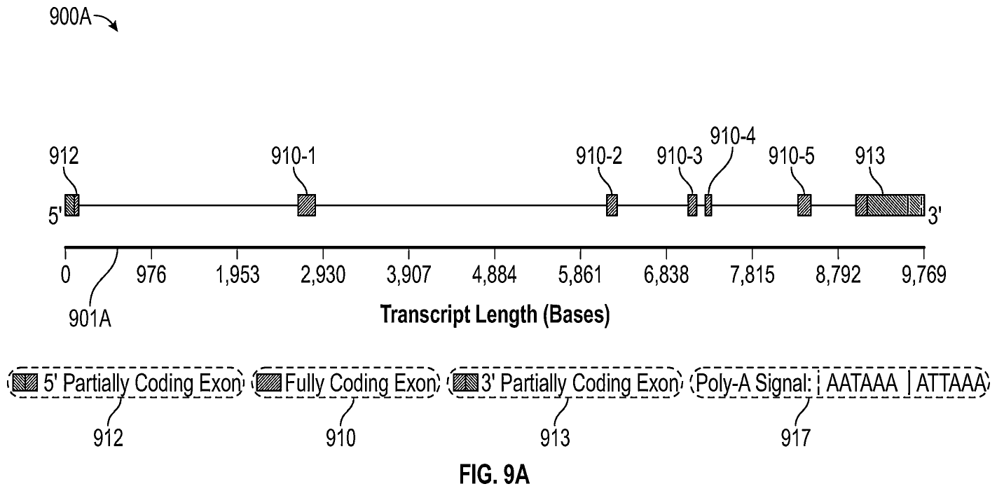

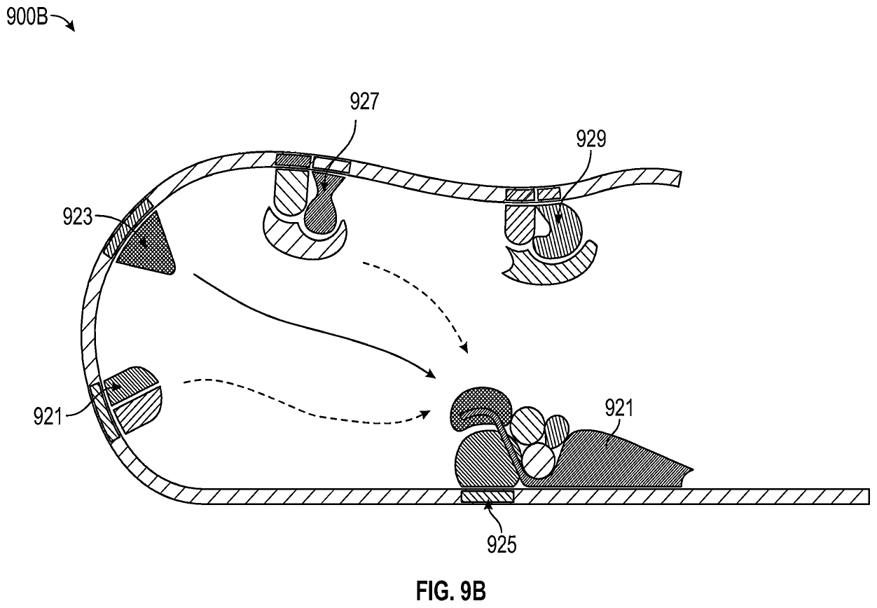

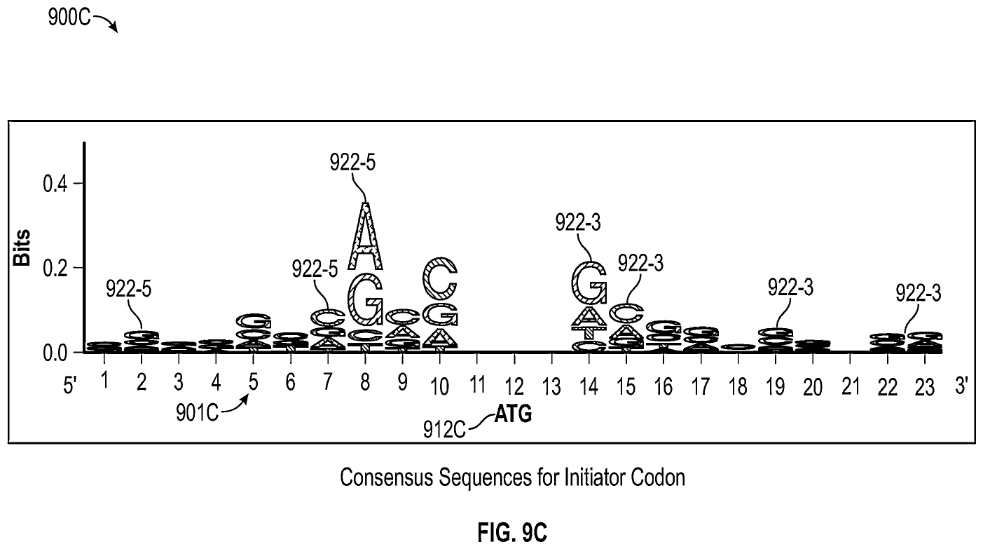

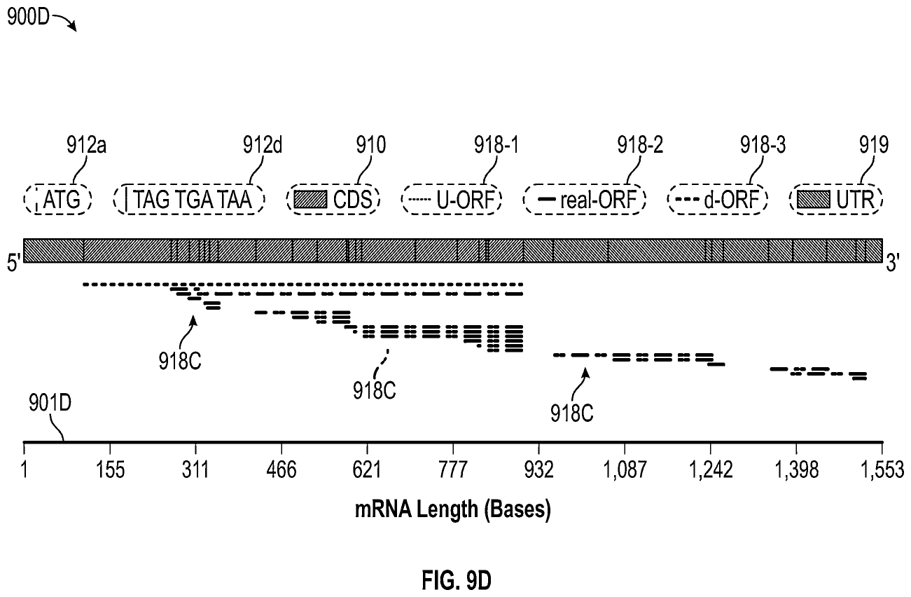

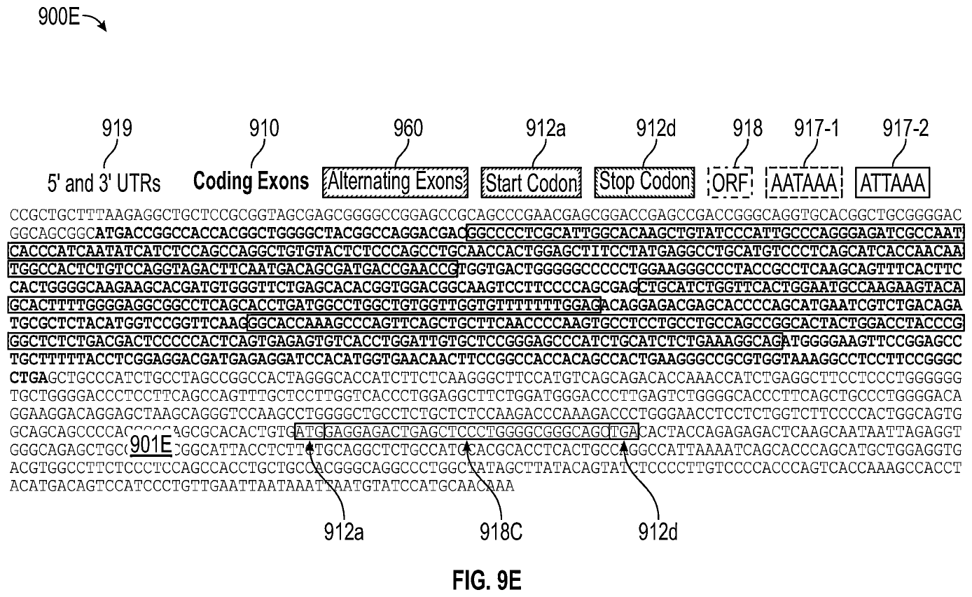

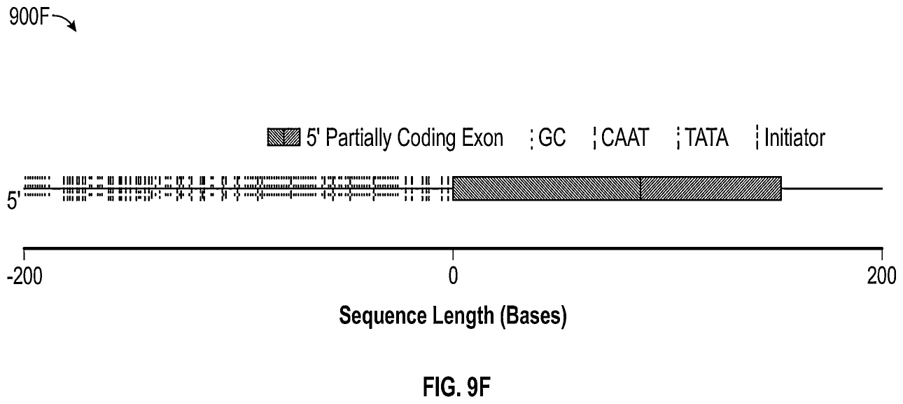

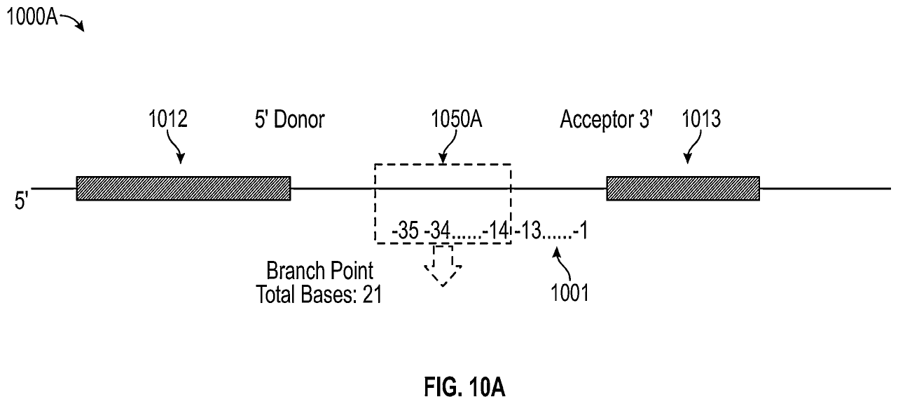

FIGS. 9A-9F illustrate exemplary embodiments of un-translated portions 900A, 900B, 900C, 900D, 900E, and 900F of a genome (hereinafter, collectively referred to as “UTRs 900”), according to embodiments disclosed herein. UTR 900A includes a nucleotide string 901A having a partially-coding exon 912, fully coding exons 910-1, 910-2, 910-3, 910-4, and 910-5 (hereinafter, collectively referred to as “exons 910”), a partially-coding exon 913, and a poly-A site 917 (AATAAA or ATTAAA). UTRs 900 may be provided by a UTR view module, as disclosed herein (e.g., UTR view module 260-7).

Promoter elements such as TATA, GC, and CAAT aid in the initiation of transcription at the transcription start site (TSS). There also exist multiple protein binding sites within the upstream sequences, which can extend up to several 1000 bases. Tumor suppressor genes such as TP53 and transcription regulating genes such as OBSCN and TAF3, bind to specific sequence motifs within the promoter regions of many genes that they control. A promoter is the binding site for the basal transcriptional apparatus—RNA polymerase and its cofactors, which provides the minimum machinery necessary to allow transcription of the gene. The enhancer regions are found at a distance from the promoter, at the 5′ or 3′ sides of the gene or within introns. They are typically short stretches of DNA (˜200 bases), each made up of a cluster of even shorter sequences (e.g., 25 bases) that are the binding sites for a variety of transcription factors. These transcription factor complexes interact with the basal transcriptional machinery at the promoter to enhance (or sometimes diminish) the transcription rate of the gene. Such interactions are possible because of the flexible nature of DNA, which allows the enhancers to come close to the promoter by looping out the DNA in between.

UTRs 900 define a promoter motif as the combination of its shorter elements such as TATA, CAAT, and GC boxes. Some embodiments calculate scores for the promoter motif by combining the individual scores of the shorter elements with various weights. This score defines the strength of the promoter. The motifs for other transcriptional regulating sequences such as enhancers and silencers are also calculated similarly. The same method is applied for polyA sequences. Mutations in these motifs are recognized by the variations in these scores.

There are possibilities for the existence of cryptic versions of all of these regulatory elements such as promoters, poly-A sites, and enhancers and silencers of promoters and poly-A signals. Mutations within these cryptic sites cause aberrations that can incorrectly enhance or suppress the gene expression or translational mechanisms. UTR's 900 thus enable a comprehensive understanding of various elements including promoters, UTRs, poly-A sites, and their cryptic sites, and their interplay with splicing and gene expression.

FIG. 9A illustrates UTR 900A, according to some embodiments.

FIG. 9B illustrates UTR 900B including a promoter motif 925 as the combination of its shorter elements such as TATA, CAAT, and GC boxes. UTR 900B illustrates other enhancer elements 921 and 927, and silencer elements 923 and 929 that interact with promoter motif 925 to activate and engage as element 921 (e.g., RNA polymerase). In some embodiments, the UTR view module calculates the scores for promoter motif by combining the individual scores of the shorter elements with various weights. This score defines the strength of promoter motif 925. Promoter motifs 925 for other transcriptional regulating sequences such as enhancer elements 921 and 927 and silencer elements 923 and 929 are calculated similarly. The same method is applied for poly-A sites 917. Mutations in promoter motifs 925 are recognized by the variations in these scores. UTRs 900 identify real and cryptic promoter and poly-A sites 917 and elements by adapting and modifying relevant algorithms (e.g., algorithm 250, including MaxEntScan, NNSplice, and Human splicing Finder) by using appropriate PWMs, consensus sequences, and lengths of the different motifs and elements. The UTR view module also uses these modified algorithms to detect mutations throughout the genes in the genome and its application to subject and cohort genomics.

Poly-A sites 917 present at the end of the coding sequence aid in the transport of mRNA molecules from the nucleus to the cytoplasm where the translation process is initiated. There exist some elements upstream and downstream of poly-A sites 917 acting as enhancers of polyadenylation. For example, a polyadenylation signal (PAS) may be placed 10-30 bases upstream of poly-A site 917, including a canonical sequence element, AATAAA. A T/GT-rich downstream sequence element (DSE) may be located up to 30 bases downstream of poly-A site 917, and T-rich upstream sequence elements (TSE), located upstream of poly-A site 917. G-rich auxiliary downstream elements (Aux-DSE) may be located downstream of the DSE, and TGTA motifs that may be found around a poly-A site 917. The secondary structure information of the amino acids 807, may act as enhancers of polyadenylation. Mutations in poly-A sites 917 and the above enhancer elements suppress the polyadenylation and affect the translation process by inhibiting mRNA transport and other translational regulation.

FIG. 9C illustrates UTR view 900C including a start codon 912C (‘ATG’) for an mRNA, with the Kozak consensus score (Y-axis) for sequences 922-5 upstream (5′ end to the left) and 922-3 downstream (3′ end to the right) of start codon 912C. The Kozak score for each motif is illustrated using their modified versions based on their consensus sequences and lengths. For each position 901C in UTR view 900C, the Kozak score is indicated for any permutation of the corresponding nucleic acid (A, C, G, T′).

The user can search for genes based on various criteria, whereby the corresponding UTR view plot and its features for the selected gene are computed and displayed for interactive analysis. In some embodiments, UTR view module provides multiple search criteria to analyze the features of UTR in genes including a search by gene. Accordingly, the user may search genes based on the gene symbols from the dropdown. In some embodiments, UTR view module provides a search criterion by the number of ORFs. In some embodiments, the search criterion is based on the number of u-ORFs, d-ORFs, and ORFs ranging from 1 to >300 that are present in the gene. In some embodiments, UTR view module provides a search by promoter box: Based on the type and number of promoter sequences such as TATA, GC, and CAAT that are present in the gene. In some embodiments, UTR view module provides a search by promoter score: Based on the calculated scores for promoter sequences such as TATA box, GC box, CAAT box, initiator box score, and average promoter score (for the complete promoter motif) that are present in the gene. In some embodiments, a UTR view module provides a search by poly-A signal: Based on the occurrence of poly-A sequence such as AATAAA or ATTAAA, or AATAAA and ATTAAA present in the gene. In some embodiments, UTR view module provides a search by exon classes: Based on the exon classifications such as 5′ exons, 3′ exons, intron-less, and internal exons present in the genes. In some embodiments, UTR view module provides a search by clinical association: The disease association of somatic cancer, germline cancer, inherited disorders, industrial panels, ACMG panels, DMG panels, and other possible panel sources are enabled in the dropdown list. In some embodiments, UTR view module provides a search by exception genes: Genes that exhibit a rare characteristic exon behavior such as an in-frame stop codon, selenocysteine codon, or no stop codons present in the end of CDS.

FIG. 9D illustrates UTR view 900D including a graphic payload result from a query built by the user from various dropdown lists enabled by the UTR view module, as described above. The results may be fetched from the database (e.g., one or more third party databases) and presented in the form of a gene view. UTR view 900D facilitates the identification of pre-splicing and post-splicing events in transcription and translation of CDS 910, UTRs 919, u-ORF 918-1, real-ORF 918-2, d-ORF 918-3 (hereinafter, collectively referred to as “ORFs 917”), and poly-A sequences that are depicted in the gene and mRNA structure plot with respect to nucleotide string 901D. ORFs 918 are delimited by a start codon 912a and one of three stop codons 912d (‘TAG,’ ‘TGA,’ or ‘TAA’).

ORFs 918 are classified into different classes based on their position (upstream, or downstream, ‘d’) with respect to the true start codon 912a and stop codon 912d, (4 u-ORFs, and 4 d-ORFs). Accordingly, u-ORFs 918-1 are defined as a sequence from an ATG that precedes the real start codon 912a to an in-frame stop codon 912d that precedes or follows the real start codon 912a. A d-ORF 918-3 is defined as a sequence from an ATG that follows real start codon 912a to an in-frame stop codon 912d that precedes or follows real stop codon 912d. ORFs 918, promoters and poly-A signals 917 occurring in the gene transcript are represented as per the color-coded schema. Upon clicking a u-ORF 918-1 or a d-ORF 918-3, the corresponding sequence is highlighted in the mRNA sequence view in addition to the 5′ and 3′ UTR, promoter, coding exons, start codon 912a and stop codon 912d, poly-A sites 917, an d-ORF 918C, with color codes and popup window details.

FIG. 9E illustrates UTR view 900E, including a nucleotide sequence 901E of a UTR section of a nucleotide string, with overlaid mutations according to a third party database source (e.g., ClinVar, dbSNP, and COSMIC), and are overlaid on these promoters, 5′ UTR and 3′ UTR elements 919 such as Kozak sequence, u-ORFs, and d-ORFs (ORFs 918), and poly-A sites 917-1 and 917-2 along with their clinical significance. Mutations from a subject genome and cohort genomes can also be visualized on UTR view 900E. A d-ORF 918C limited by start codon 912a and stop codon 912d is also indicated. In some embodiments, UTR view 900E may also include alternating exons 960.

Scores for Kozak sequences and the 4-base stop codons are also determined based on an algorithm (e.g., algorithm 250 including a Shapiro & Senapathy algorithm) and may be illustrated/tabulated together with UTR view 900E. Various tabs showing details of mRNA sequence, splice sites, and promoters are provided which enables the analysis of various UTR elements through interactive graphics and tables. The cis and trans-acting enhancers of genes, their binding proteins, and their interplay in complex gene regulation, are also predicted using the identification of the target sequences of these motifs and elements, and their aberration in disease.

A modified S&S algorithm as disclosed herein predicts the promoter boxes (e.g., TATA box, CAT box, GC box, initiator box) upstream of the gene. We found that it produces unique patterns for scores above 50, 60, 70, etc. for different score ranges. It also produces unique patterns of different promoter boxes upstream of a specific gene. We also observed that some of these patterns such as the GC boxes correspond with the G-quadruplex DNA structure. It is observed that mutation in G-quadruplex enhances the promoter strength and causes overexpression of the gene. For example, a C-KIT gene promoter mutation causes overexpression of the tyrosine kinase enzyme leading to cancer. A drug called Gleevec has been successfully developed to inhibit the kinase to treat Gastrointestinal stromal tumor (GIST). Thus, the unique repetitive GC box patterns produced by Genome Explorer will aid in the recognition of clinically significant mutations.

UTR view can also recognize mutations that weaken the promoter strength and cause under expression of the gene. This approach applies to enhancers and silencers of promoters, and polyA sites or signals and their cryptic versions, in a broad range of several thousand bases upstream and downstream of the gene, and within the gene.

In this C-KIT gene example, the field targets to inhibit the overexpressed tyrosine kinase activity for drug development. Using Splice Atlas, we can also target to mask the predicted GC box mutation(s) through RNA interference technologies such as siRNA and RNA-i. By adjusting the dose of the interference RNA, we can control the over or under expression, thus leading to the cure of the cancer. By inhibiting the silencer activity, we can enhance the expression of a gene and vice versa (by using the enhancer). This unique approach of Splice Atlas will aid in the development of drugs for cancers and other diseases.

FIG. 9F illustrates UTR view 900F, which shows 200 bases sequence upstream of the C-KIT gene wherein the different promoter boxes are color coded. The repeated GC box pattern (blue ticks) occurs for modified S&S scores of above 50.

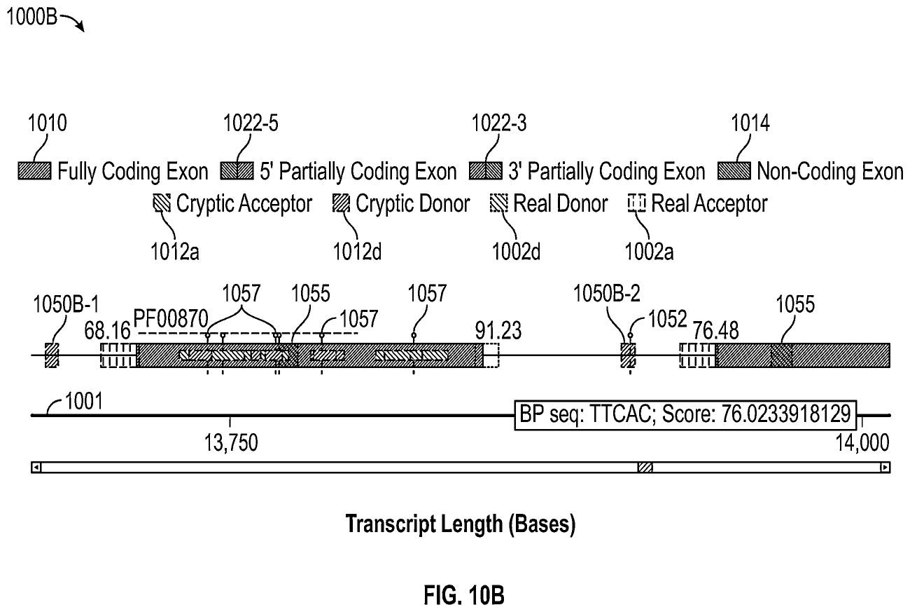

FIGS. 10A-10B illustrate exemplary branch point views 1000A and 1000B (hereinafter, collectively referred to as “branch point views 1000”) of branch point sequences (BPS) 1050A, 1050B-1, and 1050B-2 in a genome (hereinafter, collectively referred to as BPS 1050), according to embodiments disclosed herein. Introns are non-coding sequences found within the pre-mRNA transcripts that are removed during the splicing process. Splicing of pre-mRNA is assisted by the spliceosome, which identifies specific sequence motifs for the recognition of splice sites within the introns. Introns contain a donor splice site 1012 in their 5′ end, and an acceptor splice site 1013 in their 3′ end. In some embodiments, BPS 1050 may be located anywhere from 15 to 40 nucleotides upstream from the 3′ end of an intron. BPS 1050 is a highly conserved splicing signal for spliceosome assembly and lariat formation. In some embodiments, BPS 1050 is a five base regulatory sequence that may contain an Adenine at its fourth base. Accordingly, the spliceosome first cleaves the pre-mRNA at donor splice site 1012 following the attachment of an snRNP (U1) to its complementary sequence within the intron. The free end binds with BPS 1050 downstream through pairing of a G nucleotide from the 5′ end of U1 and an Adenine from BPS 1050, forming a loop known as a ‘lariat,’ releasing the intron as an RNA lariat, and covalently combining the two exons from upstream and downstream the ‘looped’ intron.

In some embodiments, BPS 1050 may be identified by using an algorithm (e.g., algorithm 250, including a modified Shapiro & Senapathy algorithm and other relevant algorithms) parsing the nucleotide string 1001 in the intron sequences upstream of 3′ end. In some embodiments, the algorithm is configured to identify a cryptic BPS 1050 within the gene. Accordingly, some embodiments provide a database for different BPS 1050s in the genome.

FIG. 10A illustrates branch point view 1000A, according to some embodiments.

FIG. 10B illustrates branch point view 1000B including a fully coding exon 1010 having a 5′ partially-coding end 1022-5 and a 3′ partially-coding end 1022-3. A non-coding exon 1014 may also be identified. Coding exon 1010 is delimited by a true acceptor 1002a and a true donor 1002d. Cryptic donor 1012d and cryptic acceptor 1012a are also identified. Branch point view 1000B illustrates a sliding window 1052 of variable sizes (e.g., 5 bases: ‘TTCAC’) and is applied on the stripped sequence from 14 to 35 bases upstream of the 3′ intron end. All possible occurrences of 5-mers (for instance) are identified and their scores are calculated (e.g., based on the PWM). Among all the 5-mers, the one with the highest score (and also above a selected threshold, e.g., 50) is considered as BPS 1050B-1 or BPS 1050B-2 (hereinafter, collectively referred to as “BPS 1050B”). Also, BPS 1050 is identified throughout the intron sequences, exons, and the complete gene and are named as cryptic branch points using the same method. When the scores of each of the 5-mers are lower than a selected threshold, the stripped sequence is again searched for the first occurrence of “A” from the 3′ end (e.g., from −14 to −35 bases). If an “A” is found, it is considered as the consensus A of BPS 1050B (4th base), three bases upstream (e.g., ‘AGC’), and one base downstream of that A (e.g., ‘G’) are then included in BPS 1050B. For example, “A” may occur at the −22 position, and thus the branch point sequence identified is “AGCAG.” There may be a few recognizable species of BPS 1050B around the non-canonical A base which can be identified and isolated based on a variety of signal identifying methodologies.

Branch point view 1000B also illustrates cryptic branch points 1055. The scores and the branch point sequence for each of the identified real and cryptic sites are shown on mouse hover. The mutations 1057 from the database sources such as dbSNP, ClinVar, and COSMIC occurring on the branch sites, cryptic branch sites, splice sites, and cryptic splice sites are shown. On clicking any of the exons, introns, or mutations 1057, the corresponding position in the expanded view automatically scrolled to focus. This enables the visualization and analysis of the various regulatory elements and their cryptic versions on the gene or transcript. Cryptic branch points 1055 may have an impact in disease associations on encountering mutations within them. Thus, the BPS view module enables the visualization and deeper analysis of BPS 1050 and other regulatory elements and their cryptic versions, individually and in combinations, in a single application.

The BPS view platform may provide search capabilities for the user according to different search criteria such as a gene basis, to search genes by entering gene symbols. In some embodiments, the search criteria may include a number of cryptic branch points, to search genes that contain a high frequency of cryptic branch point sites. In some embodiments, the search criteria may include a cryptic branch point score, to search genes that contain the highest (or one of the higher) cryptic branch point scores. In some embodiments, the search criteria may include a clinical association, to search genes based on various disease panels, a drug metabolizing gene (DMG) panel, and the American College of Medical Genetics and Genomics (ACMG) gene panel. In some embodiments, the search criteria may include an exception gene, to visualize a BPS in genes which fall under the following criteria: (i) Contains an in-frame stop codon: Displays genes having stop codons in the reading frame; (ii) Contains a selenocysteine: Displays genes having selenocysteine (an unusual amino acid), and (iii) Contains no stop codon: Displays genes having no stop codon at the end of CDS.

In some embodiments, a platform as disclosed herein enables a search for enhancers and silencers for any gene based on several search criteria such as a number of enhancers/silencers to search genes that contain a high frequency of enhancers/silencers above a score of a pre-selected value (e.g., 70 or the highest). Search criteria may include an enhancers/silencers score, to search genes that contain high enhancers/silencers scores (e.g., the highest). Search criteria may include a gene, to search genes by entering gene symbols. Search criteria may include a clinical association, to search genes based on various disease panels, a drug metabolizing gene (DMG) panel, and the American College of Medical Genetics and Genomics (ACMG) gene panel. Search criteria may include an exception gene, to visualize the protein signature of the genes which falls under the following criteria: (i) Contains an in-frame stop codon: Displays genes having stop codons in the reading frame; (ii) Contains a selenocysteine: Displays genes having selenocysteine (unusual amino acid); and (iii) Contains no stop codon: Displays genes having no stop codon at the end of CDS.

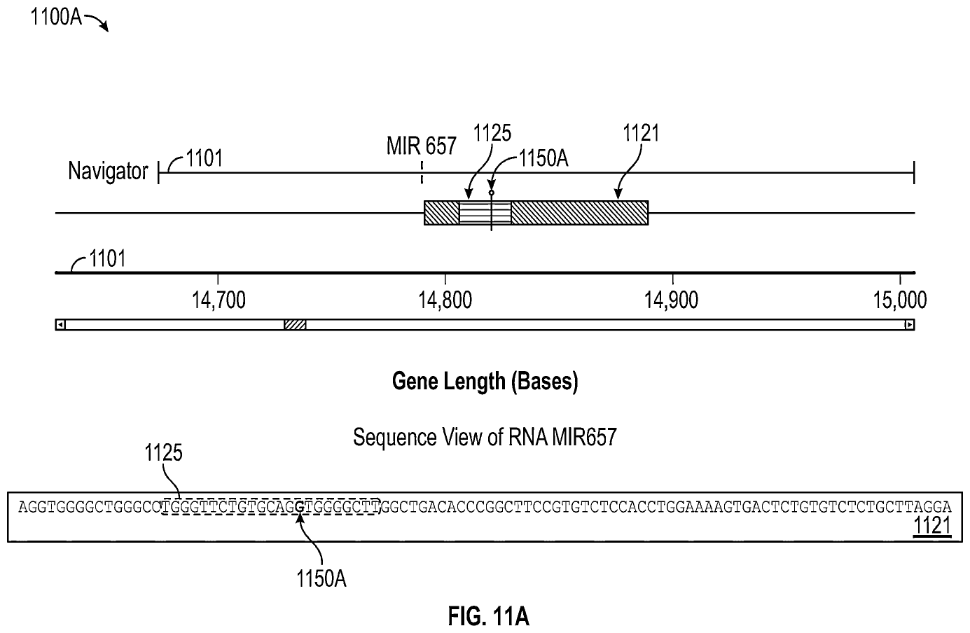

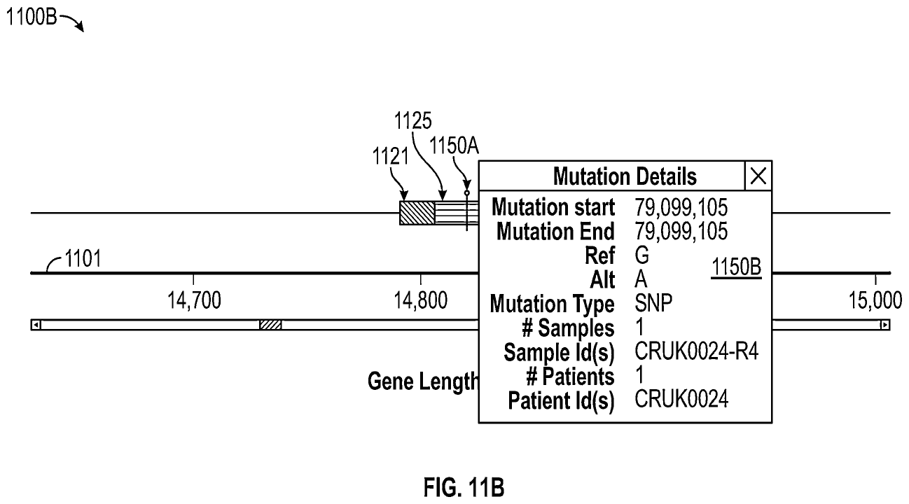

FIGS. 11A-11B illustrate exemplary embodiments of non-coding RNA genes 1100A and 1100B (hereinafter, collectively referred to as “ncRNA genes 1100”), according to embodiments disclosed herein. The ncRNA genes 1100 may be provided by an ncRNA map module as disclosed herein (e.g., ncRNA map module 260-10).

The ncRNA genes from the genome are identified based on available annotations. Graphical representation of tRNA, rRNA, miRNA, snoRNA, snRNA, and lncRNA genes in the ncRNA map is achieved by incorporating a dedicated database. Sequence information for these ncRNA genes and their exons are retrieved from SpliceDB and the graphical representation of ncRNAs are implemented. Known mutations from the data sources such as dbSNP, COSMIC, and ClinVar are depicted within the ncRNA genes in the corresponding positions. In addition, mutations from individual subjects and cohorts of subjects are also overlaid on the gene plot. The effect of mutations on the ncRNAs (such as defects in a tRNA leading to incorrect amino acid incorporation into proteins, or defects in miRNA gene leading to suppression of a specific gene expression or translation) are also predicted using the indigenous algorithm of the ncRNA map module. Furthermore, identification of ncRNA genes overlapping with the protein-coding genes is performed by comparing the coordinates of ncRNA and protein-coding genes.

TABLE IV

| | Number of genes | Sequence length |
|---|---|---|
| ncRNA type | in the genome | (spliced exons) |
| rRNA | 19 | 100-1,600 |
| tRNA | 447 | 59-86 |
| miRNA | 1,500 | 16-27 |
| snoRNA | 388 | 33-350 |
| snRNA | 95 | 63-332 |

There exists variability in these ncRNAs, for instance, a specific tRNA across multiple organisms, which helps in predicting the pathogenicity of a variant from a subject. When a mutated base falls in the non-allowed region, the structure/function of the RNA molecule is greatly altered whereas if it falls within the allowed set, the structure/function of the RNA is not altered or slightly altered. Signatures for each type of the ncRNA genes are constructed by considering the non-redundant bases in each of the positions of the aligned ncRNA sequences from various organisms. The variable and invariable positions from the ncRNA signatures are also identified. The effect of mutations are computed based on the allowed/non-allowed bases from the signature of the specific ncRNA genes.

The ncRNA map module may include a search engine that enables the user to search for portions of a nucleotide string in a subject genome according to a menu of criteria. In some embodiments, the criteria may include an ncRNA gene, to visualize splicing events for individual transcripts for the selected gene. The criteria may also include the type of ncRNA, to search and visualize specific types of non-coding RNA genes. The criteria may include a clinical association, to search and visualize splicing events for individual transcripts in genes implicated in ncRNA gene panels for all major cancers and inherited disorders. The criteria may include overlapped genes, wherein coordinates of the RNA genes are checked to identify whether they overlap with any of the genes (protein-coding) present and the overlapping genes are illustrated. The criteria may include a number of cryptic sites, to identify genes having a high frequency of cryptic splice sites that can be searched based on the number of cryptic sites. Cryptic splice sites can be visualized for individual transcripts for the selected gene. The criteria may include a cryptic site score to identify genes having high cryptic splice site scores that can be searched based on the scores (with options for >70, >80, and >90 to choose from). The cryptic splice sites can be visualized for individual transcripts for the selected gene.

The ncRNA genes 1100 are plotted on nucleotide string 1101 along the gene length depicting exons and introns within them. These genes overlapping with the protein-coding ones are also highlighted. The ncRNA genes are plotted on the scale of the gene length depicting exons and introns within them. These genes overlapping with the protein coding ones are also highlighted. The sequences of these ncRNA genes are also provided for further analysis. The mutations from the publicly available databases, and the genomes of patients and cohorts, are marked on the ncRNA gene view and the sequence view as well. The effect of mutations on the ncRNA genes are predicted and visualized for deeper analysis. There are possibilities of existence of cryptic splice sites, promoters, enhancers, and silencers for these ncRNA genes which are also identified using the modified S&S and other relevant algorithms and visualized on the gene view and sequence view. The mutations on these cryptic regulatory sites from known data sources, individual patients and cohorts of patients are visualized on the gene and sequence view.

FIG. 11A illustrates a specific sequence 1121 of ncRNA gene 1100A that may be provided for further analysis. A mutation 1150A is indicated within sequence 1125. In some embodiments, mutation 1150A is identified from a third party database, and the genomes of subjects and cohorts. The effect of mutation 1150A on ncRNA gene 1100A may be predicted and visualized for deeper analysis by the ncRNA map module, and provided in the graphic payload upon a mouse over by the user.

FIG. 11B illustrates nc RNA map 1100B, including pop up window 1150B. There are possibilities of existence of cryptic splice sites, promoters, enhancers, and silencers for these ncRNA genes which are also identified using an algorithm (e.g., algorithm 250 including a modified Shapiro & Senapathy algorithm and other relevant algorithms) and visualized on the gene view and sequence view. The nucleotide variability within the ncRNA genes may be displayed as stacks to form signatures. Mutations within the ncRNA gene can be visualized in these signatures, and pathogenicity, and their disease associations can be analyzed.

A database coupled to the ncRNA map module (e.g., database 252) includes desirable details such as sequence annotation, splice sites, cryptic splice sites, promoter, branch points, poly-A, and known mutation information. In some embodiments, the database may include information for regulatory and splicing elements such as promoters, UTRs, splice donor, acceptor and branch points, poly-A sites, enhancers and silencers of gene regulation, and splicing from different data sources (e.g., NCBI, PFAM, PfamScan, Ensembl, PDB, UniProt, ClinVar, dbSNP, COSMIC, Variant Effect Predictor, PolyPhen, SIFT), and added scores for each of these elements based on modified Shapiro & Senapathy and other relevant algorithms, and accumulated these information for genes in the human genome into a unified database. In addition, the database may include the positions and sequences of the cryptic versions of each of the regulatory and splicing elements throughout each of the genes and integrate them into this database. Furthermore, the database includes accumulated information from intergenic regions from the whole human genome. In some embodiments, the database is designed to search for a subject mutation and overlay them on the gene structure and sequence.

In addition, various types of ncRNA genes are predicted within the dark matter genome using several prediction algorithms such as tRNAscan-SE, tRNA-DL, miRDB, miRIAD, LncFinder, and PLAIDOH. We will use multiple tools for each type of ncRNAs to ensure that any genuine ncRNA genes are not missed. We will also use our proprietary algorithms to identify these ncRNAs based on the variable sequence matrix specific for each type of ncRNA that are split into shorter variable sequence signatures.

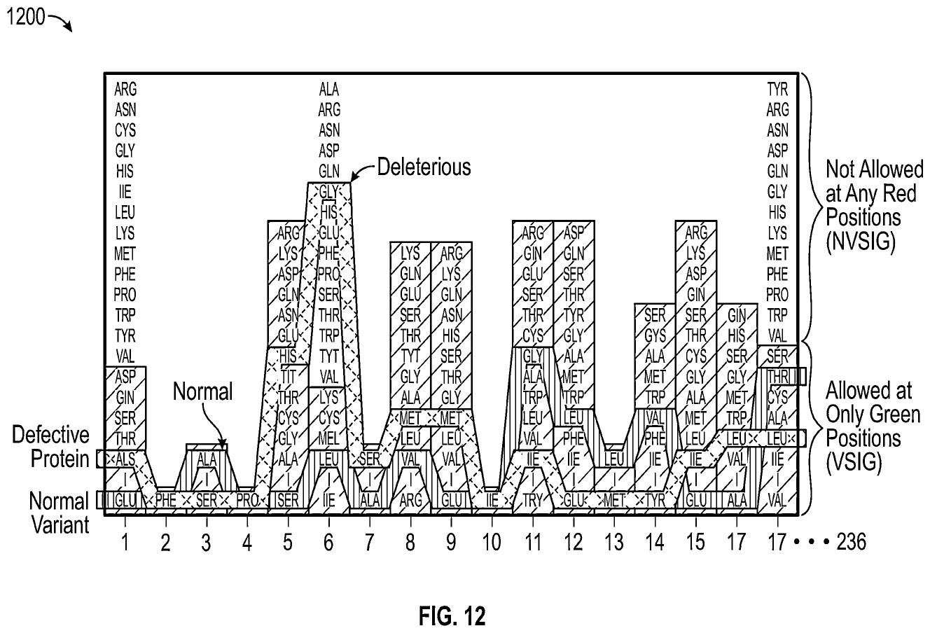

FIG. 12 illustrates a process 1200 for finding a variable and a non-variable sequence signature of a protein, according to some embodiments. The variable amino acids sequence signature of a domain from many different organisms is based on their MSA. We now have come up with a method to obtain the variable sequence signature of the domain using protein sequence from the same organism. This approach has several advantages: 1) it avoids unknown gaps that arise from multiple organisms; 2) there are many unique orphan proteins and domains present in different organisms. These orphan domains are missed in the MSA from multiple organisms. However, when we align the same protein sequence from numerous individuals of the same organism, it will lead to discovery of new domains that are not possible from the MSA of multiple organisms.

The new domains will be demarcated by a variability that is the characteristic of the genuine domains in which highly variable, invariable, and low variable AAs will be present in a recognizable manner. Mutations can also be detected and correlated with disease and drug response phenotypes using all the genetic elements of the gene.

Currently, a construct of the PWM of splicing and regulatory elements based on each type of element from a given organism. We now have come up with a method to obtain the variable sequence signature of a specific type of element, for example a donor, in a specific exon in a specific gene (e.g., TP53, exon 3, donor) by MSA of the same donor from numerous individuals.

Process 1200 may enable the discovery of unidentified elements. For example, promoter sequences are yet to be identified clearly in many genes, especially within multiple binding sites for regulatory proteins. However, when we align the genome sequence from numerous individuals of the same organism, it will lead to the discovery of new promoters, poly-A sites and signals, enhancers, silencers, and binding sites (and other elements) for binding regulatory proteins from the multiple sequence alignment. The MSA of the genome sequences of numerous individuals shows less variable positions in the binding sites compared to other positions that helps in the identification of new binding sites that are not possible to discover from the generic approach. It also helps to identify the mutations in these elements from an individual more easily, as it will show up as a rare variation (e.g., 0.0001%), as to be an outlier, that can be easily recognized.
USPACE=20×20×20=8,000 AA sequences
VSPACE=2×4×3=24 sequences
NVSPACE=USPACE−VSPACE=8,000−24=7,976 AA sequences
[2 4 3 Trp Glu Asp Ser Ala Arg Phe Gly Tyr]→VSPACE
   VSIG=AA group 1 (Phe, Ser)—AA group 2 (Gly, Ala, Glu, Trp)—AA group 3 (Tyr, Arg, Asp)

The variable and non-variable sequence signature of the λ repressor protein. (A) The allowed AAs (green) and non-allowed AAs (red) at each position of a 17-AA sequence portion of λ repressor (as experimentally determined) represent the VSIG and NVSIG of the protein. Even one AA change at a single position that diverges from the allowed AAs will make the protein defective. (B) The VSPACE, NVSPACE, and USPACE of a protein (the example shows a sequence of three AAs). The USPACE is the set of many possible sequences created by the combination of many of the twenty AAs at each sequence position. The VSPACE is defined as the set of AA sequences formed by every combination of the allowed AAs at each position. The NVSPACE is the USPACE−VSPACE.

The figure shows how the Amino Acid Sequence Variability (AAV) is constructed experimentally. We have described an algorithm for the construction of the variable amino acids sequence signature of a domain from many different organisms based on their pattern of multiple sequence alignment (MSA) in this disclosure. It has the difficulties of introducing sequence gaps and possible erroneous amino acids at some positions. We describe here a method to obtain the variable sequence signature of a domain using the protein sequences from different individuals of the same organism. This approach has several advantages: 1) it avoids unknown gaps that arise from multiple organisms, 2) it avoids sequence errors, and 3) it is expected to predict many unique orphan proteins and domains that are present in an organism that are not present in the other 108 distinct organisms. These orphan domains are missed in the multiple sequence alignment from multiple organisms. However, when we align the same protein sequence from numerous individuals of the same organism, it will lead to discovery of new domains and proteins that are not possible from the MSA of multiple organisms, and defining the AAV of these new domains in the process.

The new domains will be defined by a variability that is characteristic of the genuine domains in which highly variable, invariable, and low variable AAs will be present in a recognizable manner. Mutations can also be detected and correlated with disease and drug response phenotypes using many of the genetic elements of the gene, and the +ve and -ye AAV signatures that has been defined above.

This approach identifies new proteins and domains in groups of distinct organisms each consisting of similar species, such as mammals, crustaceans, or mollusks, or different groups of plants.

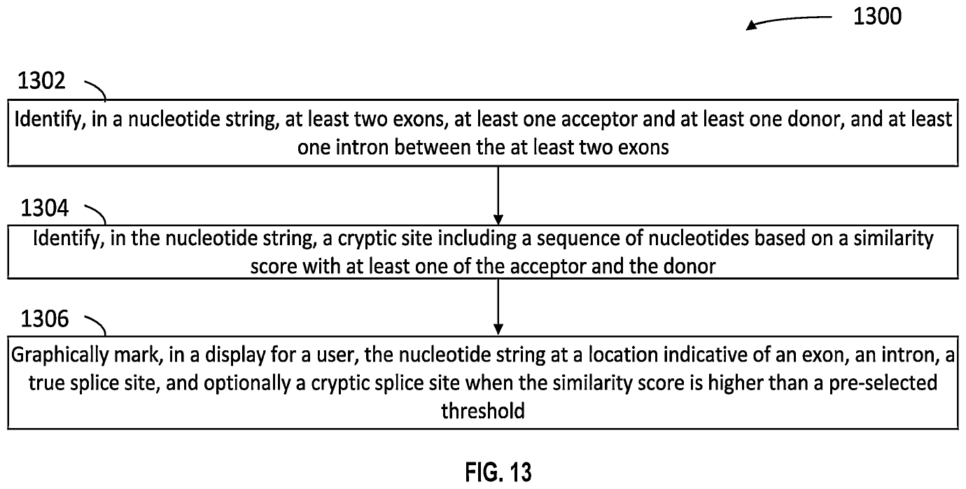

FIG. 13 is a flowchart illustrating steps in a method 1300 for identifying and displaying a cryptic site in a nucleotide string, according to some embodiments. Each one or more of the steps in method 1300 may be performed at least partially by a processor executing instructions stored in a memory of a client device or a server communicatively coupled with each other via communications modules accessing a network, as disclosed herein (e.g., processors 212, memories 220, communications modules 218, client device 110, and server 130). In some embodiments, at least one or more of the steps in method 1300 may be performed by an application hosted by the server and installed in the client device, the application including a graphic display for illustrating the results of at least one or more of the steps in method 1300 (e.g., application 222 and graphic display 225). In some embodiments, method 1300 may be at least partially performed by a genome sequence analysis engine in the server, the genome sequence analysis engine including a sequence scoring tool, a mutation tool, a statistics tool, and an algorithm tool (e.g., genome sequence analysis engine 242, sequence scoring tool 244, mutation tool 246, statistics tool 248, and algorithm 250). Further, in some embodiments, one or more of the steps in method 1300 may be performed by an exon splice module, a cryptic splice module, an exon chart module, an alternative splice module, an exon frame module, a protein signature module, a UTR view module, a BPS view module, a regulatory module, an ncRNA map module, and a dark matter module interacting with the genome sequence analysis engine, consistent with the present disclosure (e.g., modules 260). In some embodiments, a method consistent with the present disclosure may include at least one of the steps in method 1300 performed in any order, simultaneously with one another, quasi-simultaneously, or overlapping in time.

Step 1302 includes identifying, in a nucleotide string, at least two exons, at least one acceptor, at least one donor, and at least one intron between the at least two exons. In some embodiments, step 1302 includes identifying, in the nucleotide string, a first exon that lacks the acceptor and contains the donor, and identifying, in the first exon, an open reading frame between a start codon for a gene and the donor. In some embodiments, step 1302 includes identifying, in the nucleotide string, a last one exon that contains the acceptor and lacks the donor, and identifying an open reading frame between the acceptor and a terminator codon for a gene. In some embodiments, step 1302 includes identifying, in the nucleotide string, a branch point within the intron, the branch point being associated with a splicing site of the nucleotide string to combine the two exons. In some embodiments, step 1302 includes identifying, in a nucleotide string, a mutation, wherein the mutation includes a modification in at least one of the two exons, the intron, the acceptor or the donor, and optionally a branch point, and graphically marking, in the display for the user, the mutation in the nucleotide string. In some embodiments, step 1302 includes identifying, within an exon or the intron, a splice enhancer including a binding site for a spliceosome enhancer factor that promotes a splicing of exons of a gene, wherein the gene includes at least a portion of the exon and the intron. In some embodiments, step 1302 includes identifying, within an exon or the intron, a splice silencer site including a binding site for an inhibitor factor that suppresses a splicing of exons of a gene, wherein the gene includes at least a portion of the exon and the intron. In some embodiments, step 1302 includes determining a deleteriousness score of a mutation of the true splice site or the cryptic splice site based on the similarity score. In some embodiments, step 1302 includes determining the similarity score by executing instructions from an algorithm selected from a group consisting of a Shapiro & Senapathy algorithm, a MaxEntScan algorithm, and NNSplice algorithm, stored in a memory. In some embodiments, step 1302 includes identifying, in the nucleotide string, a cryptic exon that includes at least one cryptic acceptor and one cryptic donor, and optionally, an open reading frame, between the cryptic acceptor and the cryptic donor, when a cryptic splice site score is higher than a pre-selected threshold, and a length of the cryptic exon conforms to a pre-selected threshold. In some embodiments, step 1302 includes optionally identifying a cryptic branch point upstream of the cryptic exon.

Step 1304 includes identifying, in the nucleotide string, a cryptic site including a sequence of nucleotides based on a similarity score with at least one of the acceptor and the donor.

Step 1306 includes graphically marking, in a display for a user, the nucleotide string at a location indicative of an exon, an intron, a true splice site, and optionally a cryptic splice site when the similarity score is higher than a pre-selected threshold.

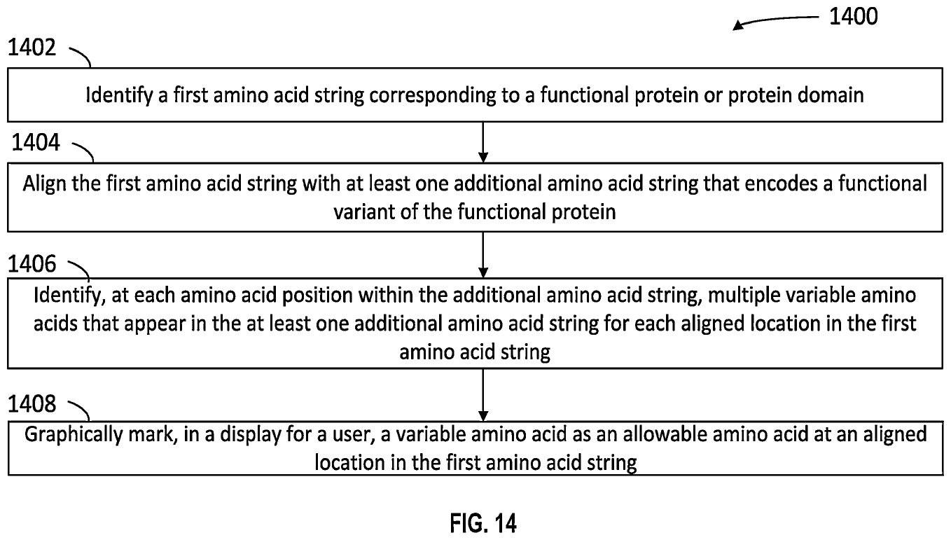

FIG. 14 is a flowchart illustrating steps in a method 1400 for creating and displaying a protein signature in an amino acid string, according to some embodiments. Each one or more of the steps in method 1400 may be performed at least partially by a processor executing instructions stored in a memory of a client device or a server communicatively coupled with each other via communications modules accessing a network, as disclosed herein (e.g., processors 212, memories 220, communications modules 218, client device 110, and server 130). In some embodiments, at least one or more of the steps in method 1400 may be performed by an application hosted by the server and installed in the client device, the application including a graphic display for illustrating the results of at least one or more of the steps in method 1400 (e.g., application 222 and graphic display 225). In some embodiments, method 1400 may be at least partially performed by a genome sequence analysis engine in the server, the genome sequence analysis engine including a sequence scoring tool, a mutation tool, a statistics tool, and an algorithm tool (e.g., genome sequence analysis engine 242, sequence scoring tool 244, mutation tool 246, statistics tool 248, and algorithm 250). Further, in some embodiments, one or more of the steps in method 1400 may be performed by an exon splice module, a cryptic splice module, an exon chart module, an alternative splice module, an exon frame module, a protein signature module, a UTR view module, a BPS view module, a regulatory module, an ncRNA map module, and a dark matter module interacting with the genome sequence analysis engine, consistent with the present disclosure (e.g., modules 260). In some embodiments, a method consistent with the present disclosure may include at least one of the steps in method 1400 performed in any order, simultaneously with one another, quasi-simultaneously, or overlapping in time.

Step 1402 includes identifying a first amino acid string corresponding to a functional protein or protein domain. In some embodiments, step 1402 includes identifying an amino acid that is different from an allowable amino acid as a disallowed amino acid at the aligned location. In some embodiments, step 1402 includes identifying, in a nucleotide string, a positive signature when the nucleotide string codes an allowed amino acid in the functional protein, and a negative signature when the nucleotide string codes a non-allowed amino acid in the functional protein. In some embodiments, step 1402 includes graphically marking a mutation of the nucleotide string on the positive signature and the negative signature. In some embodiments, step 1402 includes optionally determining a deleterious effect of the mutation based on whether the mutation occurs within the positive signature or the negative signature. In some embodiments, step 1402 includes identifying, in a nucleotide string coding a protein domain in the functional protein, a mutation leading to a disallowed amino acid, and determining a mutated hydropathy signature of the protein domain based on a hydropathy of a mutated amino acid. In some embodiments, step 1402 includes determining a normal hydropathy signature of the protein domain based on a hydropathy of an allowed amino acid or a disallowed amino acid and determining a deleteriousness score for the mutation based on a difference between the mutated hydropathy signature of the protein domain and the normal hydropathy signature of the protein domain. In some embodiments, step 1402 includes determining a deleteriousness score for the mutation based on whether a mutation occurs within a positive signature indicating no deleteriousness or a negative signature indicating a deleteriousness.

Step 1404 includes aligning the first amino acid string with at least one additional amino acid string that encodes a functional variant of the functional protein.

Step 1406 includes identifying, at each amino acid position within the additional amino acid string, multiple variable amino acids that appear in the at least one additional amino acid string for each aligned location in the first amino acid string.

Step 1408 includes graphically marking, in a display for a user, a variable amino acid as an allowable amino acid at an aligned location in the first amino acid string. In some embodiments, step 1408 includes stacking a non-redundant amino acid at each position of the additional amino acid string in the functional protein. In some embodiments, step 1408 includes graphically distinguishing, in the display for the user, the allowed amino acid and a disallowed amino acid at each aligned location. In some embodiments, step 1408 includes graphically indicating a hydropathy of each variable amino acid at each aligned location.

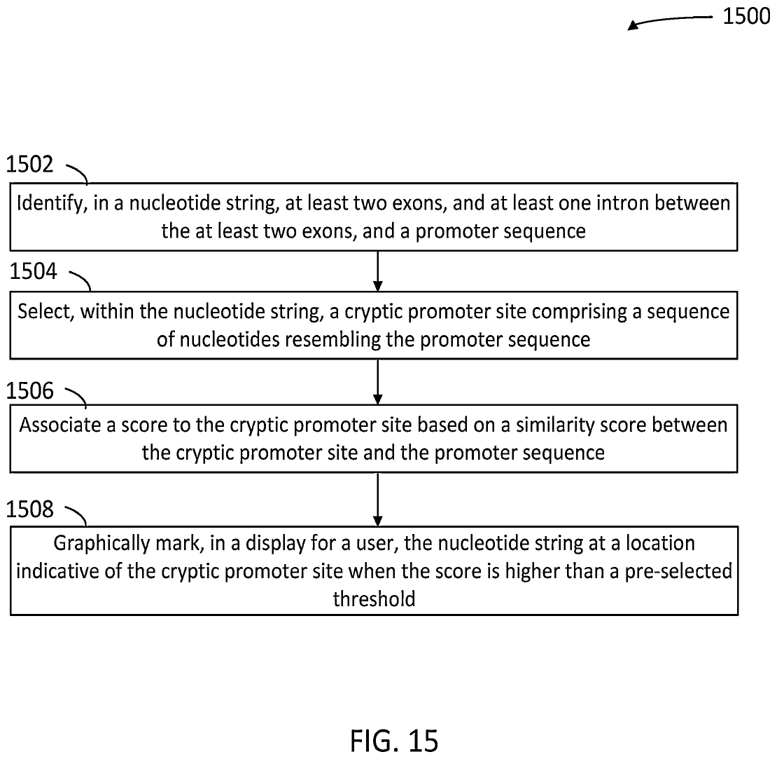

FIG. 15 is a flowchart illustrating steps in a method 1500 for identifying and displaying a cryptic promoter site in a nucleotide string, according to some embodiments. Each one or more of the steps in method 1500 may be performed at least partially by a processor executing instructions stored in a memory of a client device or a server communicatively coupled with each other via communications modules accessing a network, as disclosed herein (e.g., processors 212, memories 220, communications modules 218, client device 110, and server 130). In some embodiments, at least one or more of the steps in method 1500 may be performed by an application hosted by the server and installed in the client device, the application including a graphic display for illustrating the results of at least one or more of the steps in method 1500 (e.g., application 222 and graphic display 225). In some embodiments, method 1500 may be at least partially performed by a genome sequence analysis engine in the server, the genome sequence analysis engine including a sequence scoring tool, a mutation tool, a statistics tool, and an algorithm tool (e.g., genome sequence analysis engine 242, sequence scoring tool 244, mutation tool 246, statistics tool 248, and algorithm 250). Further, in some embodiments, one or more of the steps in method 1500 may be performed by an exon splice module, a cryptic splice module, an exon chart module, an alternative splice module, an exon frame module, a protein signature module, a UTR view module, a BPS view module, a regulatory module, an ncRNA map module, and a dark matter module interacting with the genome sequence analysis engine, consistent with the present disclosure (e.g., modules 260). In some embodiments, a method consistent with the present disclosure may include at least one of the steps in method 1500 performed in any order, simultaneously with one another, quasi-simultaneously, or overlapping in time.

Step 1502 includes identifying, in a nucleotide string, at least two exons, and at least one intron between the at least two exons, and a promoter sequence. In some embodiments, identifying the promoter sequence in step 1502 includes identifying at least one of a TATA box, a CAAT box, a GC box, and an initiator box. In some embodiments, identifying the promoter sequence in step 1502 includes identifying a TATA box, CAAT box, GC box, and initiator box, and, in addition, enhancers and silencers. In some embodiments, step 1502 includes determining the similarity score by executing instructions from an algorithm selected from a group consisting of a Shapiro & Senapathy algorithm, a MaxEntScan algorithm, and NNSplice algorithm, stored in a memory.

Step 1504 includes selecting, within the nucleotide string, a cryptic promoter site including a sequence of nucleotides resembling the promoter sequence.

Step 1506 includes associating a score to the cryptic promoter site based on a similarity score between the cryptic promoter site and the promoter sequence. In some embodiments, the similarity score includes a combination of one or more of a TATA box, a CAAT box, a GC box, and an initiator box. In some embodiments, step 1506 includes determining the similarity score by executing instructions from an algorithm selected from a group consisting of a Shapiro & Senapathy algorithm, a MaxEntScan algorithm, and NNSplice algorithm, stored in a memory.

Step 1508 includes graphically marking, in a display for a user, the nucleotide string at a location indicative of the cryptic promoter site when the score is higher than a pre-selected threshold.

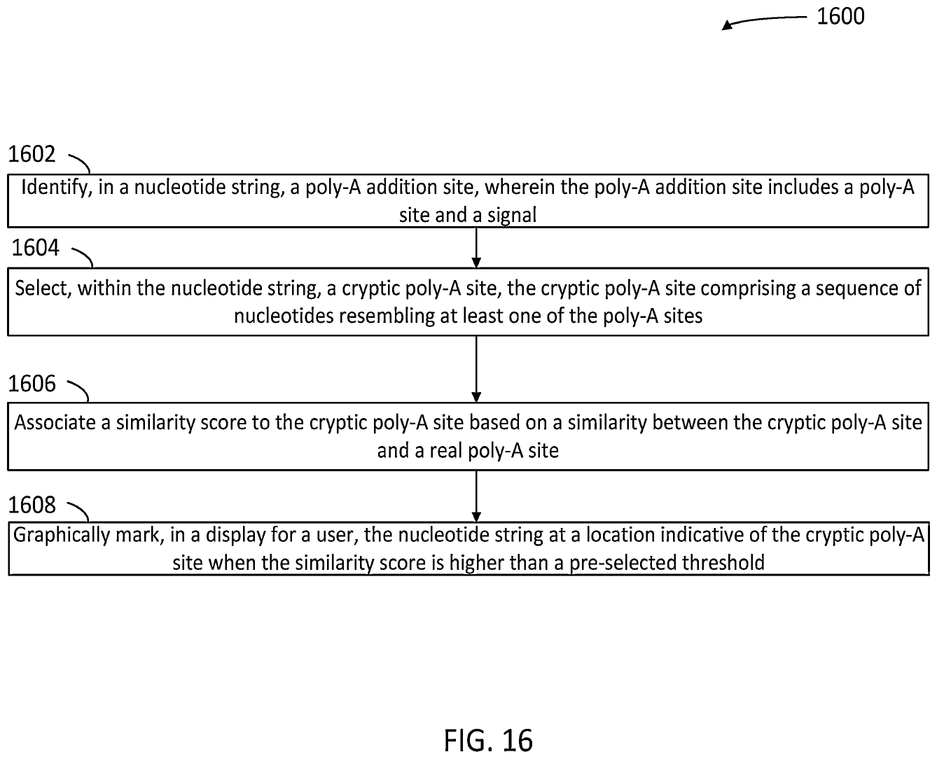

FIG. 16 is a flowchart illustrating steps in a method 1600 for identifying and displaying a cryptic poly-A site in a nucleotide string, according to some embodiments. Each one or more of the steps in method 1600 may be performed at least partially by a processor executing instructions stored in a memory of a client device or a server communicatively coupled with each other via communications modules accessing a network, as disclosed herein (e.g., processors 212, memories 220, communications modules 218, client device 110, and server 130). In some embodiments, at least one or more of the steps in method 1600 may be performed by an application hosted by the server and installed in the client device, the application including a graphic display for illustrating the results of at least one or more of the steps in method 1600 (e.g., application 222 and graphic display 225). In some embodiments, method 1600 may be at least partially performed by a genome sequence analysis engine in the server, the genome sequence analysis engine including a sequence scoring tool, a mutation tool, a statistics tool, and an algorithm tool (e.g., genome sequence analysis engine 242, sequence scoring tool 244, mutation tool 246, statistics tool 248, and algorithm 250). Further, in some embodiments, one or more of the steps in method 1600 may be performed by an exon splice module, a cryptic splice module, an exon chart module, an alternative splice module, an exon frame module, a protein signature module, a UTR view module, a BPS view module, a regulatory module, an ncRNA map module, and a dark matter module interacting with the genome sequence analysis engine, consistent with the present disclosure (e.g., modules 260). In some embodiments, a method consistent with the present disclosure may include at least one of the steps in method 1600 performed in any order, simultaneously with one another, quasi-simultaneously, or overlapping in time.

Step 1602 includes identifying, in a nucleotide string, a poly-A addition site, wherein the poly-A addition site includes a poly-A site and a signal. In some embodiments, step 1602 includes identifying a signal that includes a nucleotide string that signals an appearance of the poly-A site near the signal. In some embodiments, step 1602 includes identifying, in the nucleotide string, an enhancer of a poly-A site. In some embodiments, step 1602 includes identifying, in the nucleotide string, a silencer of a poly-A site.

Step 1604 includes selecting, within the nucleotide string, a cryptic poly-A site, the cryptic poly-A site including a sequence of nucleotides resembling at least one of the poly-A sites.

Step 1606 includes associating a similarity score to the cryptic poly-A site based on a similarity between the cryptic poly-A site and a real poly-A site. In some embodiments, step 1606 includes determining the similarity score by executing instructions from an algorithm selected from a group consisting of a Shapiro & Senapathy algorithm, a MaxEntScan algorithm, and NNSplice algorithm, stored in a memory.

Step 1608 includes graphically marking, in a display for a user, the nucleotide string at a location indicative of the cryptic poly-A site when the similarity score is higher than a pre-selected threshold. In some embodiments, step 1608 includes graphically marking in the display for the user, a real poly-A site.

### Hardware Overview

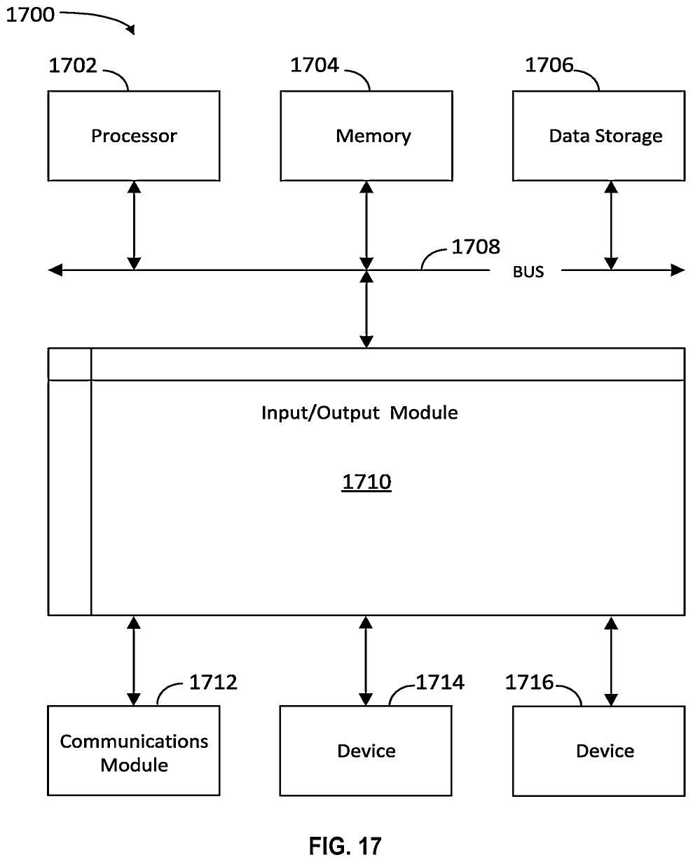

FIG. 17 is a block diagram illustrating an example computer system with which the client and server of FIGS. 1 and 2 and the methods of FIGS. 13-16 can be implemented. In certain aspects, the computer system 1700 may be implemented using hardware or a combination of software and hardware, either in a dedicated server, or integrated into another entity, or distributed across multiple entities.

Computer system 1700 (e.g., client device 110 and server 130) includes a bus 1708 or other communication mechanism for communicating information, and a processor 1702 (e.g., processors 212) coupled with bus 1708 for processing information. By way of example, the computer system 1700 may be implemented with one or more processors 1702. Processor 1702 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1700 can include, in addition to hardware, code that creates an execution environment for the computer program in question, e.g., code that constitutes processor firmware, a protocol stack, a database management system, an operating system, or a combination of one or more of them stored in an included memory 1704 (e.g., memories 220), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled with bus 1708 for storing information and instructions to be executed by processor 1702. The processor 1702 and the memory 1704 can be supplemented by, or incorporated in, special purpose logic circuitry.

The instructions may be stored in the memory 1704 and implemented in one or more computer program products, e.g., one or more modules of computer program instructions encoded on a computer-readable medium for execution by, or to control the operation of, the computer system 1700, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multi paradigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 1704 may also be used for storing temporary variable or other intermediate information during execution of instructions to be executed by processor 1702.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and inter-coupled by a communication network. The processes and logic flows described in this specification can be performed by one or more programmable processors executing one or more computer programs to perform functions by operating on input data and generating output.

Computer system 1700 further includes a data storage device 1706 such as a magnetic disk or optical disk, coupled with bus 1708 for storing information and instructions. Computer system 1700 may be coupled via input/output module 1710 to various devices. Input/output module 1710 can be any input/output module. Exemplary input/output modules 1710 include data ports such as USB ports. The input/output module 1710 is configured to connect to a communications module 1712. Exemplary communications modules 1712 (e.g., communications modules 218) include networking interface cards, such as Ethernet cards and modems. In certain aspects, input/output module 1710 is configured to connect to a plurality of devices, such as an input device 1714 (e.g., input device 214) and/or an output device 1716 (e.g., output device 216). Exemplary input devices 1714 include a keyboard and a pointing device, e.g., a mouse or a trackball, by which a user can provide input to the computer system 1700. Other kinds of input devices 1714 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices 1716 include display devices, such as an LCD (liquid crystal display) monitor, for displaying information to the user.

According to one aspect of the present disclosure, the client device 110 and server 130 can be implemented using a computer system 1700 in response to processor 1702 executing one or more sequences of one or more instructions contained in memory 1704. Such instructions may be read into memory 1704 from another machine-readable medium, such as data storage device 1706. Execution of the sequences of instructions contained in main memory 1704 causes processor 1702 to perform the process steps described herein. One or more processors in a multi-processing arrangement may also be employed to execute the sequences of instructions contained in memory 1704. In alternative aspects, hard-wired circuitry may be used in place of or in combination with software instructions to implement various aspects of the present disclosure. Thus, aspects of the present disclosure are not limited to any specific combination of hardware circuitry and software.

### RECITATION OF EMBODIMENTS

The subject technology is illustrated, for example, according to various aspects described below. Various examples of aspects of the subject technology are described as numbered embodiments. These are provided as examples, and do not limit the subject technology.

Embodiment I: a computer-implemented method includes identifying, in a nucleotide string, at least two exons, at least one acceptor, at least one donor, and at least one intron between the at least two exons, identifying, in the nucleotide string, a cryptic splice site including a sequence of nucleotides based on a similarity score with at least one of the acceptor or the donor, and graphically marking, in a display for a user, the nucleotide string at a location indicative of an exon, an intron, a true splice site, and optionally a cryptic splice site when the similarity score is higher than a pre-selected threshold.

Embodiment II: a computer-implemented method includes identifying a first amino acid string corresponding to a functional protein or protein domain, aligning said first amino acid string with at least one additional amino acid string that encodes a functional variant of said functional protein, identifying, at each amino acid position within said additional amino acid string, multiple variable amino acids that appear in the at least one additional amino acid string for each aligned location in the first amino acid string, and graphically marking, in a display for a user, a variable amino acid as an allowable amino acid at an aligned location in said first amino acid string.

Embodiment III: a computer-implemented method includes identifying, in a nucleotide string, at least two exons, and at least one intron between the at least two exons, and a promoter sequence, selecting, within the nucleotide string, a cryptic promoter site including a sequence of nucleotides resembling the promoter sequence, associating a score to the cryptic promoter site based on a similarity score between the cryptic promoter site and the promoter sequence, and graphically marking, in a display for a user, the nucleotide string at a location indicative of the cryptic promoter site when the score is higher than a pre-selected threshold.

Embodiment IV: a computer-implemented method includes identifying, in a nucleotide string, a poly-A addition site, wherein the poly-A addition site includes a poly-A site and a signal, selecting, within the nucleotide string, a cryptic poly-A site, the cryptic poly-A site including a sequence of nucleotides resembling at least one of the poly-A sites, associating a similarity score to the cryptic poly-A site based on a similarity between the cryptic poly-A site and a real poly-A site, and graphically marking, in a display for a user, the nucleotide string at a location indicative of the cryptic poly-A site when the similarity score is higher than a pre-selected threshold.

Embodiment V: a computer-implemented method including identifying a first nucleotide string corresponding to a functional non-coding RNA gene and aligning said first nucleotide string with at least one additional nucleotide string that specifies a functional variant of said ncRNA gene. The computer-implemented method includes identifying, at each nucleotide position within said additional nucleotide string, multiple variable nucleotides that appear in the at least one additional nucleotide string for each aligned location in the first nucleotide string and graphically marking, in a display for a user, a variable nucleotide as an allowable nucleotide at an aligned location in said first nucleotide string.

Embodiment VI: a computer-implemented method including identifying a first nucleotide string corresponding to a non-coding RNA gene and aligning said first nucleotide string with at least one additional nucleotide string that specifies a functional variant of said non-coding RNA gene. The computer-implemented method includes identifying, at each nucleotide position within said additional nucleotide string, multiple variable nucleotides that appear in the at least one additional nucleotide string for each aligned location in the first nucleotide string, and graphically marking, in a display for a user, a variable nucleotide as an allowable nucleotide at an aligned location in said first nucleotide string.

Embodiments I, II, III, IV, V, and VI may include any one of the below recited elements in any combination and number:

Element 1, further including identifying, in the nucleotide string, a first exon that lacks the acceptor and contains the donor, and identifying, in the first exon, an open reading frame between an initiator codon for a gene and the donor. Element 2, further including identifying, in the nucleotide string, a last exon that contains the acceptor and lacks the donor, and identifying an open reading frame between the acceptor and a terminator codon for a gene. Element 3, further including identifying, in the nucleotide string, a branch point within the intron, the branch point being associated with a splicing site of the nucleotide string to combine the two exons. Element 4, further including identifying, in a nucleotide string, a mutation, wherein the mutation includes a modification in at least one of the two exons, the intron, the acceptor or the donor, and optionally a branch point, and graphically marking, in the display for the user, the mutation in the nucleotide string. Element 5, further including identifying, within an exon or the intron, a splice enhancer including a binding site for a spliceosome enhancer factor that promotes a splicing of exons of a gene, wherein the gene includes at least a portion of the exon and the intron. Element 6, further including identifying, within an exon or the intron, a splice silencer site including a binding site for an inhibitor factor that suppresses a splicing of exons of a gene, wherein the gene includes at least a portion of the exon and the intron. Element 7, further including determining a deleteriousness score of a mutation of the true splice site or the cryptic splice site based on the similarity score. Element 8, further including determining the similarity score by executing instructions from an algorithm selected from a group consisting of a Shapiro-Senapathy algorithm, a MaxEntScan algorithm, and NNSplice algorithm, stored in a memory. Element 9, further including: identifying, in the nucleotide string, a cryptic exon that includes at least one cryptic acceptor and one cryptic donor, and optionally, an open reading frame, between the cryptic acceptor and the cryptic donor, when a cryptic splice site score is higher than a pre-selected threshold, and a length of the cryptic exon conforms to a pre-selected threshold; and optionally identifying a cryptic branch point upstream of the cryptic exon.

Element 10, further including identifying an amino acid that is different from an allowable amino acid as a disallowed amino acid at the aligned location. Element 11, wherein graphically marking the variable amino acids includes stacking a non-redundant amino acid at each position of the additional amino acid string in the functional protein. Element 12, further including graphically distinguishing, in the display for the user, the allowed amino acid and a disallowed amino acid at each aligned location. Element 13, further including: identifying, in a nucleotide string, a positive signature when the nucleotide string codes an allowed amino acid in the functional protein, and a negative signature when the nucleotide string codes a non-allowed amino acid in the functional protein; graphically marking a mutation of the nucleotide string on the positive signature and the negative signature; and optionally determining a deleterious effect of the mutation based on whether the mutation occurs within the positive signature or the negative signature. Element 14, further including graphically indicating a hydropathy of each variable amino acid at each aligned location. Element 15, further including identifying, in a nucleotide string coding a protein domain in the functional protein, a mutation leading to a disallowed amino acid; determining a mutated hydropathy signature of the protein domain based on a hydropathy of a mutated amino acid; determining a normal hydropathy signature of the protein domain based on a hydropathy of an allowed amino acid or a disallowed amino acid; determining a deleteriousness score for the mutation based on a difference between the mutated hydropathy signature of the protein domain and the normal hydropathy signature of the protein domain; and determining a deleteriousness score for the mutation based on whether a mutation occurs within a positive signature indicating no deleteriousness or a negative signature indicating a deleteriousness.

Element 16, wherein identifying the promoter sequence includes identifying at least one of a TATA box, a CAAT box, a GC box, and an initiator box. Element 17, wherein the similarity score includes a combination of one or more of a TATA box, a CAAT box, a GC box, and an initiator box. Element 18, wherein identifying the promoter sequence includes identifying a TATA box, CAAT box, GC box, and initiator box, and, in addition, enhancers and silencers. Element 19, further including determining the similarity score by executing instructions from an algorithm selected from a group consisting of a Shapiro-Senapathy algorithm, a MaxEntScan algorithm, and NNSplice algorithm, stored in a memory. Element 20, wherein identifying a poly-A site includes identifying a signal that includes a nucleotide sequence that signals an appearance of the poly-A site near the signal. Element 21, further including graphically marking in the display for the user, a real poly-A site. Element 22, further including determining the similarity score by executing instructions from an algorithm selected from a group consisting of a Shapiro-Senapathy algorithm, a MaxEntScan algorithm, and NNSplice algorithm, stored in a memory. Element 23, further including identifying, in the nucleotide string, an enhancer of a poly-A site. Element 24, further including identifying, in the nucleotide string, a silencer of a poly-A site.

Element 25, further comprising identifying the different types of ncRNA genes in the dark matter genome using known ncRNA gene prediction algorithms and proprietary algorithms, and, further multiple algorithms for each ncRNA type so as to discover most of the genuine genes. Element 26, further comprising taking variable AA strings from different individuals of the same organism such as the human, and constructing the allowable (positive) and non-allowable (negative) signatures. Element 27, further comprising taking variable AA strings from different individuals of the same organism such as the human, and discovering new domains by the presence of highly variable, invariable, and low variable AAs similar to and characteristic of genuine domains. Element 28, further comprising determining a distinct PWM or variable sequence signature for each of the splicing elements, say donor, or other regulatory or splicing elements, based on the multiple sequence alignment of genome sequences of numerous individuals from the same organism. Element 29, further comprising predicting novel promoters, binding sites, or other regulatory and splicing elements, from the PWM and MSA of genome sequences of numerous individuals, wherein, the binding sites show less variance compared to other positions, or other statistically distinct characteristics, and determining mutations within these novel binding sites. Element 30, further comprising creating a database of all these novel elements from the genome of an organism. Element 31, further comprising determining that the invariance of the AA directly correlates with the deleteriousness of a mutation, indicating that the mutation at an invariant AA position is the most deleterious, with decreasing deleteriousness correlating with increasing amino acid variability, and applying this to determine the deleteriousness of a patient mutation. Element 32, further comprising identifying a nucleotide that is different from an allowable nucleotide as a disallowed nucleotide at the aligned location. Element 33, wherein graphically marking the variable nucleotides comprises stacking a non-redundant nucleotide at each position of the additional nucleotide string in the functional ncRNA. Element 34, further comprising graphically distinguishing, in the display for the user, the allowed and a disallowed nucleotide at each aligned location. Element 35, further comprising: identifying, in a nucleotide string, a positive signature, and a negative signature from the allowed and disallowed nucleotides; graphically marking a mutation of the nucleotide string on the positive signature and the negative signature; and optionally determining a deleterious effect of the mutation based on whether the mutation occurs within the positive signature or the negative signature. Element 36, further comprising, displaying the mutations in each of the genetic elements in each of the non-coding RNA genes, on the gene structure, depicting the processing steps of the ncRNA gene into the active element, and additionally elaborating these features in a sequence view, indicating the steps at which the processing error occurs.

Element 37, further including identifying a nucleotide that is different from an allowable nucleotide as a disallowed nucleotide at the aligned location. Element 38, wherein graphically marking the variable nucleotides includes stacking a non-redundant nucleotide at each position of the additional nucleotide string in the non-coding RNA gene. Element 39, further including graphically distinguishing, in the display for the user, the allowable nucleotide and a disallowed nucleotide at each aligned location. Element 40, further including identifying, in a nucleotide string, a positive signature, and a negative signature from the allowable nucleotide and a disallowed nucleotide; graphically marking a mutation of the nucleotide string on the positive (allowed) signature and the negative (dis-allowed) signature; and optionally determining a deleterious effect of the mutation based on whether the mutation occurs within the positive signature or the negative signature. Element 41, further including identifying a recognition sequence element in each of the non-coding RNA genes by using instructions contained in algorithms such as Shapiro-Senapathy, NNSplice, MaxEntScan, or their modified versions therefore; optionally, displaying the recognition sequence element on a gene structure, depicting the non-coding RNA gene into an active element, and additionally elaborating these features in a sequence view; and indicating a position of a sequence error. Element 42, further including displaying a mutation in the non-coding RNA gene; depicting the non-coding RNA gene in an active element; elaborating a sequence view; and indicating an error position in the sequence view. Element 43, further including taking variable AA strings from different individuals of a same organism, and constructing an allowable signature and a non-allowable signature. Element 44, further including taking variable AA strings from different individuals of a same organism; and discovering new domains by at least one of a highly variable, an invariable, a low variable AAs similar to and characteristic genuine domains, discarding a random nucleotide (the four bases) sites that indicate non-functional regions. Element 45, further including determining a distinct PWM or variable sequence signature for a splicing element, say donor, or other regulatory or the splicing element, based on a multiple sequence alignment of gene or genome sequences of numerous individuals from a same species or a group of organisms consisting of similar species. Element 46, further including predicting novel promoters, binding sites or other regulatory and splicing elements, from a PWM and an MSA of a gene sequence for multiple individuals, wherein, the binding sites show a mixture of low, medium, and high variance compared to other random nucleotide positions, or other statistically distinct characteristics indicative of functional regions, and determining mutations within these novel binding sites. Element 47, further including creating a database of many these novel elements from a genome of an organism. Element 48, further including correlating an invariance or a degree of variance of an AA pair combination with a deleteriousness of a mutation, indicating that the mutation at an invariant AA position is highly deleterious, with a decreasing deleteriousness correlating with increasing amino acid variability; and applying this to determine the deleteriousness of a patient mutation.

In one aspect, a method may be an operation, an instruction, or a function and vice versa. In one aspect, a claim may be amended to include some words (e.g., instructions, operations, functions, or components) recited in other one or more claims, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software, or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

1. A computer-implemented method, comprising:
identifying, in a nucleotide string, at least two exons, at least one acceptor, at least one donor, and at least one intron between the at least two exons;
identifying, in the nucleotide string, a cryptic splice site comprising a sequence of nucleotides based on a similarity score with at least one of the acceptor or the donor; and
graphically marking, in a display for a user, the nucleotide string at a location indicative of an exon, an intron, a true splice site, and optionally a cryptic splice site when the similarity score is higher than a pre-selected threshold.

2. The computer-implemented method of claim 1, further comprising identifying, in the nucleotide string, a first exon that lacks the acceptor and contains the donor, and identifying, in the first exon, an open reading frame between an initiator codon for a gene and the splice junction within the donor.

3. The computer-implemented method of claim 1, further comprising identifying, in the nucleotide string, a last exon that contains the acceptor and lacks the donor, and identifying an open reading frame between the splice junction within the acceptor and a terminator codon for a gene.

4. The computer-implemented method of claim 1, further comprising identifying, in the nucleotide string, a branch point within the intron, the branch point being associated with a splicing site of the nucleotide string to combine the two exons.

5. The computer-implemented method of claim 1, further comprising identifying, in a nucleotide string, a mutation, wherein the mutation comprises a modification in at least one of the two exons, the intron, the acceptor or the donor, and optionally a branch point, and graphically marking, in the display for the user, the mutation in the nucleotide string.

6. The computer-implemented method of claim 1, further comprising identifying, within an exon or the intron, a splice enhancer site comprising a binding site for a spliceosome enhancer factor that promotes a splicing of exons of a gene, wherein the gene comprises at least a portion of the exon and the intron.

7. The computer-implemented method of claim 1, further comprising identifying, within an exon or the intron, a splice silencer site comprising a binding site for an inhibitor factor that suppresses a splicing of exons of a gene, wherein the gene comprises at least a portion of the exon and the intron.

8. The computer-implemented method of claim 1, further comprising determining a deleteriousness score of a mutation of the true splice site or the cryptic splice site based on the similarity score variability.

9. The computer-implemented method of claim 1, further comprising determining the similarity score by executing instructions from an algorithm selected from a group consisting of algorithms such as Shapiro-Senapathy algorithm, a MaxEntScan algorithm, and NNSplice algorithm, stored in a memory.

10. The computer-implemented method of claim 1, further comprising determining the similarity score by executing instructions from a modified algorithm selected from a group consisting of algorithms such as Shapiro-Senapathy algorithm, a MaxEntScan algorithm, and NNSplice algorithm, stored in a memory, based on the characteristics of the sequence signals such as length and the variability.

11. The computer-implemented method of claim 1, further comprising determining the similarity score by executing instructions from an algorithm selected from a group consisting of algorithms such as Shapiro-Senapathy algorithm, a MaxEntScan algorithm, and NNSplice algorithm, stored in a memory, and further determining a combined score of these algorithms based on their average or differentially weighted scores.

12. The computer-implemented method of claim 1, further comprising:
identifying, in the nucleotide string, a cryptic exon that comprises at least one cryptic acceptor and one cryptic donor, and optionally, an open reading frame, between the cryptic acceptor and the cryptic donor, when a cryptic splice site score is higher than a pre-selected threshold, and a length of the cryptic exon conforms to a pre-selected threshold; and optionally
identifying a cryptic branch point upstream of the cryptic exon,
and/or identifying a cryptic splice enhancer site within, upstream, and/or downstream of the cryptic exon and/or intron,
and/or identifying a cryptic splice silencer site within, upstream, and/or downstream of the cryptic exon and/or intron.

13. The computer-implemented method of claim 1, further comprising determining a distinct PWM or variable sequence signature for a distinct splicing element, say donor, or other regulatory or the splicing element within a gene, based on a multiple sequence alignment of the splicing element within a gene or genome sequences of numerous individuals from a same species or a group of organisms consisting of similar species;
and, further, creating a database of many of these novel elements from a genome of an organism.

14. A computer-implemented method, comprising:
identifying a first amino acid string corresponding to a functional protein or protein domain;
aligning said first amino acid string with at least one additional amino acid string that encodes a functional variant of said functional protein;
identifying, at each amino acid position within said additional amino acid string, multiple variable amino acids that appear in the at least one additional amino acid string for each aligned location in the first amino acid string; and
graphically marking, in a display for a user, a variable amino acid as an allowable amino acid at an aligned location in said first amino acid string.

15. The computer-implemented method of claim 14, further comprising identifying an amino acid that is different from an allowable amino acid as a disallowed amino acid at the aligned location.

16. The computer-implemented method of claim 14, wherein graphically marking the variable amino acids comprises stacking a non-redundant amino acid at each position of the additional amino acid string in the functional protein.

17. The computer-implemented method of claim 14, further comprising graphically distinguishing, in the display for the user, the allowed amino acid and a disallowed amino acid at each aligned location.

18. The computer-implemented method of claim 14, further comprising:
identifying, in a nucleotide string, a positive signature when the nucleotide string codes an allowed amino acid in the functional protein, and a negative signature when the nucleotide string codes a non-allowed amino acid in the functional protein;
graphically marking a mutation of the nucleotide string on the positive signature and the negative signature; and optionally
determining a deleterious effect of the mutation based on whether the mutation occurs within the positive signature or the negative signature.

19. The computer-implemented method of claim 14, further comprising graphically indicating a hydropathy of each variable amino acid at each aligned location.

20. The computer-implemented method of claim 14, further comprising
identifying, in a nucleotide string coding a protein domain in the functional protein, a mutation leading to a disallowed amino acid;
determining a normal hydropathy signature of the protein domain based on a hydropathy of an allowed amino acid or a disallowed amino acid;
determining a mutated hydropathy signature of the protein domain based on a hydropathy of a mutated amino acid;
determining a deleteriousness score for the mutation based on a difference between the mutated hydropathy signature of the protein domain and the normal hydropathy signature of the protein domain; and
determining a deleteriousness score for the mutation based on whether a mutation occurs within a positive signature indicating no deleteriousness or a negative signature indicating a deleteriousness.

21. The computer-implemented method of claim 14, further comprising:
taking variable AA strings from different individuals of a same organism of a protein sequence; and
constructing an allowable signature and a non-allowable signature.

22. The computer-implemented method of claim 14, further comprising:
taking variable AA strings from different individuals of a same organism from intronic or intergenic regions in a genome;
constructing an variable amino acid sequence signature representing possible domains;
and identifying unknown genes, exons, and introns based on portions of variable AA signatures.

23. The computer-implemented method of claim 14, further comprising:
taking variable AA strings from different individuals of a same organism; and
discovering new domains by at least one of a highly variable, an invariable, a low variable AAs similar to and characteristic of genuine domains, discarding a random AA (the 20 AAs) sites that indicate non-functional regions by searching in all three reading frames of a nucleotide sequence.

24. The computer-implemented method of claim 14, further comprising creating a database of many of these novel domains from a genome of an organism.

25. The computer-implemented method of claim 14, further comprising:
correlating an invariance or a degree of variance of an AA pair combination with a deleteriousness of a mutation, indicating that the mutation at an invariant AA position is highly deleterious, with a decreasing deleteriousness correlating with increasing amino acid variability; and
applying this to determine the deleteriousness of a patient mutation.

26-64. (canceled)
